(19) United States
(12) Patent Application Publication
(10) Pub. No.: US 2025/0122493 A1
(43) Pub. Date: Apr. 17, 2025

(54) RECOMBINANT FACTOR IX PROTEINS, METHODS OF MAKING, AND METHODS OF USING THE SAME IN NON-HEMOPHILIC SUBJECTS

(71) Applicant: The University of North Carolina at Chapel Hill, Chapel Hill, NC (US)

(72) Inventor: Tal Kafri, Carrboro, NC (US)

(21) Appl. No.: 18/682,530

(22) PCT Filed: Aug. 12, 2022
(86) PCT No.: PCT/US2022/074889

§ 371 Date: Feb. 9, 2024

Related U.S. Application Data
(60) Provisional application No. 63/232,771, filed on Aug. 13, 2021.

(51) Int. Cl. C12N 9/64; A61K 38/00; A61K 49/00; G01N 33/86

(52) U.S. Cl. CPC C12N 9/644; A61K 49/0004; C12Y 304/21022; G01N 33/86; A61K 38/00; G01N 2333/9645

(57) ABSTRACT

This invention relates to recombinant aptamer 9.3t-resistant Factor IX proteins, as well as methods of making the same and methods of using the same in methods comprising non-hemophilic subjects, including methods of modeling Factor IX replacement therapy in a non-hemophilic subject and methods of identifying genetic background effects of a non-hemophilic subject on an exogenous Factor IX replacement protein. The invention also pertains to methods of modeling gene and/or protein replacement therapy in a protein non-deficient subject and methods of identifying genetic background effects of a protein non-deficient subject on an exogenous replacement protein, as well as methods of selecting aptamer-resistant recombinant proteins and selecting aptamer-sensitive protein non-deficient subjects for use in such methods as disclosed herein.

### STATEMENT OF GOVERNMENT SUPPORT

This invention was made with government support under Grant Nos. HL128119 and DK058702 awarded by the National Institutes of Health. The government has certain rights in the invention.

### STATEMENT REGARDING ELECTRONIC FILING OF A SEQUENCE LISTING

A Sequence Listing in XML format, entitled 5470-887WO_ST26.xml, 46,216 bytes in size, generated on Aug. 11, 2022 and filed herewith, is hereby incorporated by reference in its entirety for its disclosures.

### FIELD OF THE INVENTION

This invention relates to methods of modeling protein and/or gene replacement therapy in a protein non-deficient subject and methods of identifying genetic background effects of a protein non-deficient subject on an exogenous replacement protein, as well as methods of selecting aptamer-resistant recombinant proteins and selecting aptamer-sensitive protein non-deficient subjects for use in methods such as disclosed herein.

### BACKGROUND OF THE INVENTION

Animal model-based preclinical trials to evaluate the efficacy and safety of gene and protein replacement therapies in vivo are an obligatory phase in the process of approving the use of novel therapeutics in humans. Currently, the majority of preclinical animal models are premised on genetically knocked out (KO) rodents. A small number of human diseases can be modeled in large animal models when relevant naturally occurring genetic deficiencies in these animals are identified. The relative complexity and costs of generating and maintaining KO rodent strains limit the number of murine/rat strains in which a specific gene KO is established. As a result, the effects of the host genetic variation on the outcome of gene/protein replacement therapies cannot be studied in currently employed animal models. Several gene therapy clinical trials reported on patient-to-patient variations in efficacy/safety (Cavazzana-Calvo et al. 2010 Nature 467 (7313): 318-322; Gaspar et al. 2011 Sci Transl Med 3 (97): 97ra79; Hacein-Bey-Abina et al. 2003 Science 302 (5644): 415-419; Manno et al. 2006 Nat Med 12 (3): 342-347).

### SUMMARY OF THE INVENTION

One aspect of the invention provides an in vitro method of modeling protein and/or gene replacement therapy in a protein non-deficient subject, comprising: (i) providing a sample from one or more protein non-deficient subject comprising an endogenous target protein; (ii) contacting the sample with an effective amount of an aptamer, wherein the effective amount of the aptamer reduces activity of the endogenous target protein; and (iii) contacting the sample with an effective amount of an exogenous aptamer-resistant replacement protein.

Another aspect of the invention provides an in vitro method of identifying genetic background effects of a protein non-deficient subject on an exogenous protein, comprising the steps of: (i) providing a sample from one or more protein non-deficient subject comprising an endogenous target protein; (ii) contacting the sample with an effective amount of an aptamer, wherein the effective amount of the aptamer reduces activity of the endogenous target protein; (iii) contacting the sample with an effective amount of an exogenous aptamer-resistant replacement protein; (iv) assaying for the presence of protein activity in the sample comprising the exogenous aptamer-resistant replacement protein and in a control; (v) comparing the relative level and/or presence of protein activity of the sample comprising the exogenous aptamer-resistant replacement protein and the control to identify genetic background effects of the subject on the efficacy and/or safety of the exogenous replacement protein; and (vi) associating the difference in relative level and/or presence of the protein activity with genetic background differences between the subject and the control.

An additional aspect of the invention provides a method of modeling protein and/or gene replacement therapy in one or more protein non-deficient subject, comprising: (i) administering to the one or more subject an effective amount of an aptamer, wherein the effective amount of the aptamer reduces activity of an endogenous target protein; and (ii) administering to the one or more subject an effective amount of an exogenous aptamer-resistant replacement protein.

An additional aspect of the invention provides a method of identifying genetic background effects on an exogenous protein in one or more protein non-deficient subject, comprising the steps of: (i) administering to the one or more subject an effective amount of an aptamer, wherein the effective amount of the aptamer reduces activity of an endogenous target protein; (ii) administering to the one or more subject an effective amount of an exogenous aptamer-resistant replacement protein; (iii) assaying for the presence of protein activity in the one or more subject comprising the exogenous aptamer-resistant replacement protein and in a control; (iv) comparing the relative level and/or presence of protein activity of the one or more subject comprising the exogenous aptamer-resistant replacement protein and the control to identify genetic background effects of the subject on the efficacy and/or safety of the exogenous replacement protein; and (v) associating the difference in relative level and/or presence of the protein activity with genetic background differences between the one or more subject and the control.

Another aspect of the invention provides a method of selecting an exogenous replacement protein for modeling protein and/or gene replacement therapy in a protein non-deficient subject, comprising (i) introducing an effective amount of an aptamer to one or more sample(s) comprising an endogenous target protein of said aptamer, and then assaying for the presence of activity of the endogenous target protein in the one or more sample(s) comprising said amount of said aptamer, wherein the presence of activity in the one or more sample(s) comprising said aptamer as compared to a control sample that lacks activity (e.g., a sample not comprising said aptamer) indicates resistance of the endogenous target protein to inhibition by the aptamer and wherein the lack of activity in the one or more sample(s) comprising the aptamer indicates sensitivity of the endogenous target protein to inhibition by the aptamer; (ii) identifying the endogenous target protein comprised in the one or more sample(s) comprising an effective amount of said aptamer as sensitive to said aptamer (i.e., an aptamer-sensitive endogenous target protein), or identifying the endogenous target protein comprised in the one or more sample(s) unable to determine an effective amount of said aptamer as resistant to said aptamer (i.e., an aptamer-resistant endogenous target protein); and (iii) selecting the aptamer-resistant endogenous target protein as an exogenous (aptamer-resistant) replacement protein.

Another aspect of the invention provides a method of selecting a model subject for modeling protein and/or gene replacement therapy in a protein non-deficient subject, comprising (i) introducing an effective amount of an aptamer to one or more sample(s) comprising an endogenous target protein of said aptamer, and then assaying for the presence of activity of the endogenous target protein in the one or more sample(s) comprising said amount of said aptamer, wherein the presence of activity in the one or more sample(s) comprising said aptamer as compared to a control sample that lacks activity (e.g., a sample not comprising said aptamer) indicates resistance of the endogenous target protein to inhibition by the aptamer and wherein the lack of activity in the one or more sample(s) comprising the aptamer indicates sensitivity of the endogenous target protein to inhibition by the aptamer; (ii) identifying the endogenous target protein comprised in the one or more sample(s) comprising an effective amount of said aptamer as sensitive to said aptamer (i.e., an aptamer-sensitive endogenous target protein), or identifying the endogenous target protein comprised in the one or more sample(s) unable to determine an effective amount of said aptamer as resistant to said aptamer (i.e., an aptamer-resistant endogenous target protein); and (iii) selecting the one or more sample(s) comprising the aptamer-sensitive endogenous target protein as a model subject for modeling protein and/or gene replacement therapy in a protein non-deficient subject.

Another aspect of the invention provides a method of determining an effective amount of an aptamer for reducing activity of an endogenous target protein comprising (i) introducing a first amount of the aptamer to one or more sample(s) comprising an endogenous target protein of said aptamer, and assaying for the presence of activity of the endogenous target protein in the one or more sample(s) comprising said first amount of said aptamer, wherein the presence of activity in the one or more sample(s) comprising said aptamer as compared to a control sample that lacks activity (e.g., a sample not comprising said aptamer) indicates resistance of the endogenous target protein to inhibition by the aptamer and wherein the lack of activity in the one or more sample(s) comprising the aptamer indicates sensitivity of the endogenous target protein to inhibition by the aptamer; and then (ii) repeating step (i) with an escalating amount of the aptamer until: (a) an escalating amount of the aptamer reduces activity of the endogenous target protein in the one or more sample(s) as compared to uninhibited endogenous target protein activity, thereby determining the effective amount of the aptamer for the one or more sample, or (b) no escalating amount of the aptamer reduces activity of the endogenous target protein in the one or more sample(s) as compared to uninhibited endogenous target protein activity.

Also provided is an aptamer-resistant exogenous replacement protein as identified by the methods of the invention. In some embodiments, the aptamer-resistant exogenous replacement protein as identified by the methods of the invention may be for use as a diagnostic agent, medical imaging agent, and/or aptamer screening agent.

Also provided are isolated nucleic acid molecules, vectors, transformed cells, transgenic animals encoding and/or comprising an aptamer-resistant exogenous replacement protein of the invention.

Another aspect of the invention provides a recombinant Factor IX protein of a mammal, comprising: (a) one or more substitution(s) of an XIa cleavage site-α and/or an XIa cleavage site-β of a human Factor IX protein; and/or (b) an N-terminal substitution of a human Factor IX N-terminus amino acid segment; wherein the recombinant Factor IX protein is resistant to inhibition by aptamer 9.3t.

An additional aspect of the invention provides a method of identifying a Factor IX protein as resistant to inhibition by aptamer 9.3t, comprising: (i) introducing an effective amount of aptamer 9.3t to one or more sample(s) comprising an endogenous Factor IX protein of a mammalian subject, and then assaying for the presence of clotting activity in the one or more sample(s) comprising said effective amount of aptamer 9.3t, wherein the presence of clotting activity in the one or more sample(s) comprising aptamer 9.3t indicates resistance of the endogenous Factor IX to inhibition by aptamer 9.3t and wherein the lack of clotting activity in the one or more sample(s) comprising aptamer 9.3t indicates sensitivity of the endogenous Factor IX to inhibition by aptamer 9.3t, as compared to a control sample; and (ii) identifying the endogenous Factor IX comprised in the one or more sample(s) comprising an effective amount of aptamer 9.3t as sensitive to aptamer 9.3t (i.e., an aptamer-sensitive endogenous Factor IX), or identifying the endogenous Factor X comprised in the one or more sample(s) unable to determine an effective amount of aptamer 9.3t as resistant to aptamer 9.3t (i.e., an aptamer-resistant endogenous Factor IX).

Another aspect of the invention provides a method of determining an effective amount of aptamer 9.3t for reducing clotting activity of an endogenous Factor IX comprising (i) introducing a first amount of aptamer 9.3t to the one or more sample(s) comprising an endogenous Factor IX protein of a mammalian subject, and assaying for the presence of clotting activity in the one or more sample(s) comprising said effective amount of aptamer 9.3t, wherein the presence of clotting activity in the one or more sample(s) comprising aptamer 9.3t indicates resistance of the endogenous Factor IX to inhibition by aptamer 9.3t and wherein the lack of clotting activity in the one or more sample(s) comprising aptamer 9.3t indicates sensitivity of the endogenous Factor IX to inhibition by aptamer 9.3t, as compared to a control sample; and then (ii) repeating step (i) with an escalating amount of aptamer 9.3t until: (a) an escalating amount of aptamer 9.3t reduces activity of the endogenous Factor IX in the one or more sample(s) as compared to uninhibited endogenous Factor IX activity, thereby determining the effective amount of aptamer 9.3t for the one or more sample, or (b) no escalating amount of aptamer 9.3t reduces activity of the endogenous Factor IX in the one or more sample(s) as compared to uninhibited endogenous target protein activity.

Another aspect of the invention provides a recombinant Factor IX protein identified as resistant to aptamer 9.3t by the methods of the invention.

Also provided are isolated nucleic acid molecules, vectors, transformed cells, transgenic animals encoding and/or comprising a recombinant Factor IX protein of the invention.

Another aspect of the invention provides an in vitro method of modeling protein and/or gene replacement therapy for treating a bleeding disorder in one or more non-hemophilic subject, comprising: (i) providing a sample from one or more protein non-deficient subject comprising an endogenous Factor IX protein; (ii) contacting the sample with an effective amount of aptamer 9.3t, wherein the effective amount of the aptamer reduces activity of the endogenous Factor IX protein; and (iii) contacting the sample with an effective amount of an exogenous aptamer-resistant replacement Factor IX protein, nucleic acid molecule, vector, cell, and/or composition of the invention.

Another aspect of the invention provides an in vitro method of identifying genetic background effects of one or more non-hemophilic subject on an exogenous Factor IX protein, comprising the steps of: (i) providing a sample from one or more non-hemophilic subject comprising an endogenous Factor IX protein; (ii) contacting the sample with an effective amount of aptamer 9.3t, wherein the effective amount of the aptamer 9.3t reduces activity of the endogenous Factor IX protein; (iii) contacting the sample with an effective amount of an exogenous aptamer-resistant replacement Factor IX protein, nucleic acid molecule, vector, cell, and/or composition of the invention; (iv) assaying for the presence of protein clotting activity in the sample comprising the exogenous aptamer-resistant replacement Factor IX protein and in a control; (v) comparing the relative level and/or presence of protein clotting activity of the sample comprising the exogenous aptamer-resistant replacement Factor IX protein and the control to identify genetic background effects of the subject on the efficacy and/or safety of the exogenous replacement Factor IX protein; and (vi) associating the difference in relative level and/or presence of the protein clotting activity with genetic background differences between the one or more subject and the control.

Another aspect of the invention provides a method of modeling a Factor IX protein and/or gene replacement therapy for treating a bleeding disorder in one or more non-hemophilic subject, comprising: (i) administering to the one or more subject an effective amount of aptamer 9.3t, wherein the effective amount of aptamer 9.3t reduces activity of endogenous Factor IX protein; (ii) administering to the one or more subject an effective amount of an exogenous aptamer-resistant replacement Factor IX protein, nucleic acid molecule, vector, cell, and/or composition of the invention.

An additional aspect of the invention provides a method of identifying genetic background effects on an exogenous Factor IX protein in one or more non-hemophilic subject, comprising the steps of: (i) administering to the one or more subject an effective amount of aptamer 9.3t, wherein the effective amount of aptamer 9.3t reduces activity of endogenous Factor IX protein; (ii) administering to the one or more subject an effective amount of an exogenous aptamer-resistant replacement Factor IX protein, nucleic acid molecule, vector, cell, and/or composition of the invention; (iii) assaying for the presence of clotting activity in the subject comprising the exogenous Factor IX protein and in a control; (iv) comparing the relative presence of clotting activity of the one or more subject comprising the exogenous Factor IX protein and the control to identify genetic background effects of the subject on the efficacy of the exogenous Factor IX protein; and (v) associating the difference in relative level and/or presence of the clotting activity with genetic background differences between the one or more subject and the control.

Further aspects, features and advantages of this invention will become apparent from the detailed description of the embodiments which follow.

### BRIEF DESCRIPTION OF THE DRAWINGS

FIG. 1 shows a plot of an activated partial thromboplastin time assay (aPTT) plotting clotting time against increased concentrations of 9.3t aptamer (0.5-4.0 μM). Assays were conducted with normal plasma obtained from eight species including Canine (open triangle), Guineapig (inverted open triangle), Hamster (closed triangle), Human (open square), Simian (X), Murine (open circle), Porcine (open diamond) and Rabbit (closed square) in the presence of increasing concentrations of the 9.3t aptamer. aPTT values in seconds were determined in triplicate.

FIGS. 2A-2B show a schematic of the structure of different lentiviral vectors employed to deliver the wild type and modified FIX proteins to HepG2 and 293T cells and a data graph of measured vector and protein specific activities. FIG. 2A shows a schematic of the physical map of lentiviral vector constructs comprising FIX cDNAs. The internal CMV promoter that regulates FIX expression, the internal ribosome entry site (IRES) that facilitates translation of the GFP/blasticidin selection marker are indicated. The names of the vector cassettes (pTK1335, 1835, pTK1847, pTK1904) carrying the cognate cDNAs are outlined in a box outside the vector map. FIG. 2B shows a data plot of specific activity of lentiviral vectors and FIX proteins following transduction of HepG2 and 293T cells. To determine specific activity of lentiviral vectors carrying the FIX cDNAs, VSV-g pseudotyped vector particles were employed on HepG2 and 293T cells at low MOI (<0.001). Vector transduced cells were selected in the presence of blasticidin. Vector copy number (VCN) per HepG2 and 293T cell was determined by qPCR. FIX concentration and clotting activity were determined by ELISA and aPTT assay, respectively. Vector specific activity was calculated as FIX concentration per VCN. FIX protein specific activity was calculated as FIX International Unit (IU) per FIX protein concentration.

FIG. 3 shows a data graph of clotting activity. The modified rabbit FIX encoded by cDNA c1904 exhibits efficient clotting activity in the presence of the 9.3t aptamer. aPTT assays were employed to determine the effects of aptamer 9.3t on clotting time of plasma samples obtained from various species including, canine, human, simian, murine, murine and porcine either in the absence (−) or presence (+) of purified FIX protein (4 μM) encoded by cDNA c1904.

FIG. 4 shows a schematic of lentiviral vector (pTK1924) and corresponding data plots charactering cTK1908 protein (“mrFIX”) function in vivo following lentiviral vector mediated hepatic delivery in three non-hemophilic mouse strains. The novel cTK1908 was cloned into a lentiviral vector pTK1924 under the control of a liver specific promoter. VSV-g pseudotyped lentiviral vector (pTK1924) particles were administered intraperitoneally (2.5 μg p24^{gag} per gram mouse body weight) to male and females mice of three strains, including C57BL/6J, collaborative cross strain 061, and F1 mice generated by crossing C57BL/6J with CC061 mice. mrFIX protein concentration and its clotting activity in treated mouse plasma were determined periodically by ELISA and aPTT assay, respectively. Percent clotting activity was calculated. Clotting activity of 10 μg/ml of purified mrFIX in the presence of 9.3t aptamer (8 μM) was set as a 100% reference standard. FIG. 4 panel A shows a depiction of the lentiviral vector construct pTK1924. The modified 5′ LTR comprising the CMV promoter HIV-1 R and U5 regions is shown. The HIV packaging signal (Y), Reve Response Element (RRE), the central poly-purine tract (cPPT) and the hAAT promoter are shown. The modified rabbit cDNA cTK1908 is shown. The internal ribosome entry site (IRES) and the fusion protein selection marker comprising the green fluorescence and blasticidin resistant gene (GFP-BSD) are shown. FIG. 4 panel B shows a bar graph of the concentration of plasma mrFIX in female and male mice at week 1 and 10 post vector administration. FIG. 4 panel C shows a bar graph of % clotting activity of mrFIX in plasma samples obtained from female and male mice at week 1 and 10 post vector administration. FIG. 4 panel D shows a bar graph of calculated mrFIX specific clotting activity in the mouse plasma samples. Specific clotting activity was calculated as International clotting Units (IU) per mg mrFIX.

FIG. 5 shows a sequence alignment between WT rabbit Factor IX protein sequence GenBank® Accession No. NP001182654.1 (SEQ ID NO:2), WT human Factor IX protein sequence GenBank® Accession No. AAA56822.1 (SEQ ID NO:24), Padua comprising WT human Factor IX protein sequence GenBank® Accession No. QWM97845.1 (SEQ ID NO: 25), as well as the same two human sequences lacking the first 46 amino acid residues comprising the signal peptide and protein peptide (“WThuman_AAA56822.1_minus46,” comprising residues 47-461 of SEQ ID NO:24, also noted herein as SEQ ID NO:1; and “Paduahuman_QWM97845.1_minus46,” comprising residues 47-461 of SEQ ID NO:25).

FIG. 6 shows a schematic of the design of an aptamer-resistant FIX with extended half-life. FIG. 6 panel A: Depiction of cDNA cTK2294, which encodes a novel fusion protein comprising aptamer resistant factor IX (in a yellow box) fused by a XIa cleavable linker (shown in red) to 4 Myc tags (in a brown box) and a tandem of two fragment crystallizable (Fc) regions (in green boxes) separated by a poly-glycine linker (in blue). Each Fc region comprises a hinge domain (H) and two heavy chain constant domains (CH2 and CH3). Note that a disulfide bond bridges the Hinge regions. The human XIa cleavage is indicated. FIG. 6 panel B: Depiction of the cTK2294 protein structure. Dashed lines indicate on regions encoded by the rabbit factor IX, complete lines indicate on regions encoded by human genes. Signal peptide (SP), pro-peptide (PP), light chain (LC), activated peptide (AP), heavy chain (HC). Hinge region (H), heavy chain constant domains 2 and 3 (CH2 and CH3). FIG. 6 panel C: Depiction of putative aptamer resistant factor IX-albumin fusion protein. Dashed lines indicate on regions encoded by the rabbit factor IX, complete lines indicate on regions encoded by human genes. Signal peptide (SP), pro-peptide (PP), light chain (LC), activated peptide (AP), heavy chain (HC). The human albumin protein is shown.

FIG. 7 shows data plots of pharmacokinetic and half-life (t_{1/2}) of hFIX proteins in hemophilia B mice following high dose (80 mg) administration. Hemophilia B mice were i.v. injected with WT (n=9), co-R338L (n=9) and human co-R338L-2Fc (n=8). The proteins were concentrated and purified from 293T cells transduced with lentiviral vectors carrying the respective cDNA's. Plasma sample were collected at 15 min, 8 hr and daily at 1-7 days post-injection. FIG. 7 top panel: hFIX concentration in plasma samples were determined by ELISA. FIG. 7 bottom panel: Terminal t_{1/2} of hFIX in treated mice were calculated using the SigmaPlot version 13 software.

FIGS. 8A-8B show a data plot and associated table indicating that factor IX fusion protein prTK2294 resists the inhibitory activity of aptamer 9.3t. FIG. 8A: A bar graph of the clotting activity of the human factor IX protein comprising the Padua mutation (prTK1335) and of the fusion protein comprising an aptamer resistant factor IX and a tandem of two Fc domains (prTK2294). Percent of factor IX clotting activity was determined by aPTT assay either in presence of aptamer 9.3t (open bars) or in the presence of an inactive mutant aptamer 9.3tm (black bars). FIG. 8B: A table of the percent activity of factor IX prTK1335 and prTK2294 as determined by aPTT assays in the presence of either aptamer 9.3t or aptamer 9.4tm.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of =10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 2nd Ed. (Cold Spring Harbor, NY, 1989); Ausubel et al. Current Protocols in Molecular Biology (Green Publishing Associates, Inc. and John Wiley & Sons, Inc., New York).

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such sub combination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

As used herein, “transduction” of a cell by a virus vector (e.g., a lentiviral vector) means entry of the vector into the cell and transfer of genetic material into the cell by the incorporation of nucleic acid into the virus vector and subsequent transfer into the cell via the virus vector.

Unless indicated otherwise, “efficient transduction” or “efficient tropism,” or similar terms, can be determined by reference to a suitable positive or negative control (e.g., at least about 50%, 60%, 70%, 80%, 85%, 90%, 95% or more of the transduction or tropism, respectively, of a positive control or at least about 110%, 120%, 150%, 200%, 300%, 500%, 1000% or more of the transduction or tropism, respectively, of a negative control).

Similarly, it can be determined if a virus “does not efficiently transduce” or “does not have efficient tropism” for a target tissue, or similar terms, by reference to a suitable control. In particular embodiments, the virus vector does not efficiently transduce (i.e., does not have efficient tropism for) tissues outside the liver, e.g., CNS, kidney, gonads and/or germ cells. In particular embodiments, undesirable transduction of tissue(s) is 20% or less, 10% or less, 5% or less, 1% or less, 0.1% or less of the level of transduction of the desired target tissue(s).

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), but in representative embodiments are either single or double stranded DNA sequences.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

A “nucleic acid,” “nucleic acid molecule,” or “nucleotide sequence” is a sequence of nucleotide bases, and may be RNA, DNA or DNA-RNA hybrid sequences (including both naturally occurring and non-naturally occurring nucleotide), but is preferably either single or double stranded DNA sequences.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

As used herein, by “isolate” or “purify” (or grammatical equivalents) a virus vector or virus particle or population of virus particles, it is meant that the virus vector or virus particle or population of virus particles is at least partially separated from at least some of the other components in the starting material. In representative embodiments an “isolated” or “purified” virus vector or virus particle or population of virus particles is enriched by at least about 10-fold, 100-fold, 1000-fold, 10,000-fold or more as compared with the starting material.

The term “endogenous” refers to a component naturally found in an environment, i.e., a gene, nucleic acid, miRNA, protein, cell, or other natural component expressed in the subject, as distinguished from an introduced component, i.e., an “exogenous” component.

As used herein, the term “heterologous” refers to a nucleotide/polypeptide that originates from a foreign species, or, if from the same species, is substantially modified from its native form in composition and/or genomic locus by deliberate human intervention.

A “heterologous nucleotide sequence” or “heterologous nucleic acid” is a sequence that is not naturally occurring in the virus. Generally, the heterologous nucleic acid or nucleotide sequence comprises an open reading frame that encodes a polypeptide and/or a nontranslated RNA.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., anti-cancer effects or improvement in transplant survivability or induction of an immune response.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

By “substantially retain” a property and/or to maintain a property “substantially the same” as a comparison (e.g., a control), it is meant that at least about 75%, 85%, 90%, 95%, 96%, 97%, 98%, 99% or 100% of the property (e.g., activity or other measurable characteristic) is retained.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset are substantially less than what would occur in the absence of the present invention.

A “treatment effective” or “effective” amount as used herein is an amount that is sufficient to provide some improvement or benefit to the subject. Alternatively stated, a “treatment effective” or “effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

As used herein the term “bleeding disorder” reflects any defect, congenital, acquired or induced, of cellular, physiological, or molecular origin that is manifested in bleedings. Examples are clotting factor deficiencies (e.g., hemophilia A and B or deficiency of coagulation Factors XI or VII), clotting factor inhibitors, defective platelet function, thrombocytopenia, von Willebrand's disease, or bleeding induced by surgery or trauma. The terms “nucleotide sequence of interest (NOI),” “heterologous nucleotide sequence” and “heterologous nucleic acid molecule” are used interchangeably herein and refer to a nucleic acid sequence that is not naturally occurring (e.g., engineered). Generally, the NOI, heterologous nucleic acid molecule or heterologous nucleotide sequence comprises an open reading frame that encodes a polypeptide and/or nontranslated RNA of interest (e.g., for delivery to a cell and/or subject).

As used herein, the terms “virus vector,” “vector” or “gene delivery vector” refer to a virus (e.g., lentivirus) particle that functions as a nucleic acid delivery vehicle, and which comprises a viral genome (e.g., viral DNA [vDNA]) and/or replicon nucleic acid molecule packaged within a virus particle. Alternatively, in some contexts, the term “vector” may be used to refer to the vector genome/vDNA alone.

The term “vector,” as used herein, means any nucleic acid entity capable of amplification in a host cell. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. The choice of vector will often depend on the host cell into which it is to be introduced. Vectors include, but are not limited to plasmid vectors, phage vectors, viruses or cosmid vectors. Vectors usually contain a replication origin and at least one selectable gene, i.e., a gene which encodes a product which is readily detectable or the presence of which is essential for cell growth.

As used herein, the term “amino acid” or “amino acid residue” encompasses any naturally occurring amino acid, modified forms thereof, and synthetic amino acids. Naturally occurring, levorotatory (L-) amino acids are shown in Table 1.

Conservative amino acid substitutions are known in the art. In particular embodiments, a conservative amino acid substitution includes substitutions within one or more of the following groups: glycine, alanine; valine, isoleucine, leucine; aspartic acid, glutamic acid; asparagine, glutamine; serine, threonine; lysine, arginine; and/or phenylalanine, tyrosine.

Alternatively, the amino acid can be a modified amino acid residue (nonlimiting examples are shown in Table 2) and/or can be an amino acid that is modified by post-translation modification (e.g., acetylation, amidation, formylation, hydroxylation, methylation, phosphorylation or sulfatation).

Further, the non-naturally occurring amino acid can be an “unnatural” amino acid as described by Wang et al., Annu Rev Biophys Biomol Struct. 35:225-49 (2006)).

As used herein, the terms “reduce,” “reduces,” “reduction,” “diminish,” “inhibit” and similar terms mean a decrease of at least about 5%, 10%, 15%, 20%, 25%, 35%, 50%, 75%, 80%, 85%, 90%, 95%, 97% or more.

As used herein, the terms “enhance,” “enhances,” “enhancement” and similar terms indicate an increase of at least about 25%, 50%, 75%, 100%, 150%, 200%, 300%, 400%, 500% or more.

The term “cleavage site,” “XIa cleavage site,” “cleavage site a” and/or “cleavage site B” means a site in the amino acid sequence of the Factor IX protein that is cleaved in the presence of activated Factor XI (FXIa). Nonlimiting examples of other cleavage sites that can be present in the sequence of the Factor IX recombinant protein of this invention include IIa, IXa, VIIa, Xa, XIIa, XIIIa and/or FVIIIa cleavage sites, which would be understood by one of ordinary skill in the art to include the cleavage site recognized by the respective clotting factor when said clotting factor is present in activated form (e.g., the VIIa cleavage site is recognized and cleaved by the activated form of Factor VII (FVIIa).

The term “gene therapy” refers to a method of changing the expression of an endogenous gene by exogenous administration of a gene. As used herein, “gene therapy” also refers to the replacement of a defective gene encoding a defective protein, or replacement of a missing gene, by introducing a functional gene corresponding to the defective or missing gene into somatic or stem cells of an individual in need. Gene therapy can be accomplished by ex vivo methods, in which differentiated or somatic stem cells are removed from the individual's body followed by the introduction of a normal copy of the defective gene into the explanted cells using a viral vector as the gene delivery vehicle. In addition, in vivo direct gene transfer technologies allow for gene transfer into cells in the individual in situ using a broad range of viral vectors, liposomes, protein DNA complexes or naked DNA in order to achieve a therapeutic outcome. The term “gene therapy” also refers to the replacement of a defective gene encoding a defective protein by introducing a polynucleotide that functions substantially the same as the defective gene or protein should function if it were not defective into somatic or stem cells of an individual in need.

The term “protein replacement therapy” refers to a method of treating an endogenous protein deficiency with the exogenous introduction of a protein, e.g., a replacement protein. As used herein, “replacement” also refers to the replacement of a defective gene encoding a defective protein, or replacement of a missing gene, by introducing a functional gene corresponding to the defective or missing gene or gene product (e.g., protein, e.g., Factor IX protein) into somatic or stem cells of an individual in need, and may be referred to as “protein and/or gene replacement therapy” and/or “gene replacement therapy”. Protein and/or gene replacement can be performed by a number of methods, including but not limited to, direct introduction and/or administration of the replacement protein to the sample and/or subject as well delivery of said protein via a nucleic acid molecule encoding said protein and/or an expression cassette, a vector, and/or a cell comprising said nucleic acid molecule.

The usual and customary meaning of “bioavailability” is the fraction or amount of an administered dose of a biologically active drug that reaches the systemic circulation. In the context of embodiments of the present invention, the term “bioavailability” includes the usual and customary meaning but, in addition, is taken to have a broader meaning to include the extent to which the Factor IX protein is bioactive. In the case of Factor IX, for example, one measurement of “bioavailability” is the procoagulant activity of Factor IX protein obtained in the circulation post-infusion.

“Posttranslational modification” has its usual and customary meaning and includes but is not limited to removal of leader sequence, γ-carboxylation of glutamic acid residues, β-hydroxylation of aspartic acid residues, N-linked glycosylation of asparagine residues, O-linked glycosylation of serine and/or threonine residues, sulfation of tyrosine residues, phosphorylation of serine residues and any combination thereof.

As used herein, “biological activity” is determined with reference to a standard derived from human plasma. For Factor IX, the standard is MONONINE® (ZLB Behring). The biological activity of the standard is taken to be 100%.

“Effective amount” as used herein refers to an amount of an exogenous protein, vector, nucleic acid, cell, composition, or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. In some embodiments, an effective amount may be determined, e.g., based on an iterative process comprising administering escalating doses of the exogenous protein, vector, nucleic acid, cell, composition, or formulation of the invention until a threshold effect is reached, such wherein the administered amount reduced activity of the endogenous target protein (e.g., endogenous Factor IX protein) by at least 50, 60, 70, 80, 90, 91, 92, 93, 94, 95, 96, 97, 98, 99, or 100% of uninhibited endogenous target protein activity.

A “treatment effective” amount as used herein is an amount that is sufficient to treat (as defined herein) the subject. Those skilled in the art will appreciate that the therapeutic effects need not be complete or curative, as long as some benefit is provided to the subject.

“Aptamer” as used herein refers to a single-stranded nucleic acid (RNA, DNA, or modified forms thereof) whose distinct nucleotide sequence determines the folding of the molecule into a unique three-dimensional structure. Nucleic acid aptamers typically comprise a degenerate or random sequence flanked by fixed sequences onto which primers may bind for amplification. Modified DNA and/or RNA bases may be used or incorporated as desired, e.g., beta-D-Glucosyl-Hydroxymethyluracil. See, e.g., U.S. Pat. No. 7,329,742. The nucleic acids may include any combination of naturally-occurring nucleosides (A, G, C, T, U), and/or nucleoside or nucleotide analogs and/or derivatives as are well known in the art, including cytotoxic, synthetic, rare, non-natural bases or altered nucleotide bases. In addition, a modification can be incorporated to reduce exonucleolytic degradation, such as a reverse (3′→5′) linkage at the 3′-terminus.

As used herein, the term “aptamer-resistant” and “aptamer-sensitive” refer to a protein and/or other gene product which is either resistant or sensitive to inhibition and/or other activity modulation by a cognate aptamer, e.g., Factor IX and aptamer 9.3t. Aptamer resistance by a particular protein and/or other gene product may vary in different species, strains, and/or individual subjects of different genetic backgrounds, e.g., one subject (e.g., species, strain, and/or individual subject) may comprise an aptamer-resistant protein while a different subject (e.g., species, strain, and/or individual subject) may comprise an aptamer-sensitive version of the same protein. Sensitivity and resistance may be measured by any relevant known method in the art, including but not limited to the methods as disclosed herein.

Compositions and Methods with Aptamer-Resistant Exogenous Replacement Proteins and Aptamer-Sensitive Model Subjects.

The present invention is based on the unexpected discovery of aptamer 9.3t-resistant and—sensitive Factor IX proteins, wherein the inventors of the herein disclosed invention discovered that aptamer-resistant Factor IX proteins could be delivered therapeutically to non-hemophilic hosts, wherein transient hemophilia was induced via administration of aptamer 9.3t to suppress expression and/or activity of endogenous aptamer-sensitive Factor IX. Thus, the invention allows, inter alia, the study of host genetic background effects on exogenously delivered Factor IX proteins, including parameters such as safety testing, efficacy testing, and/or screening of various protein modifications including but not limited to introduction of the Padua mutation and/or codon-optimization. Accordingly, the disclosed invention addresses the need to model gene and protein replacement therapies in a large number of genetically defined wild type (WT) small mammal model strains (e.g., rodents) as well as in large animals.

The invention is premised on synthetic single stranded DNA or RNA oligonucleotides (aptamers), which may be evolved by systematic evolution of ligands by exponential enrichment (SELEX) technique to bind and inhibit target molecule in very high specificity. Aptamers are known in the art and have been developed for a large number of therapeutic and diagnostic applications (“theranostics”), as well as for medical imaging procedures and as sensitive biosensors. To date aptamers have not been employed to establish animal models to characterize the efficacy and safety of gene and protein replacement therapies (see e.g., Kaur et al. 2018 Theranostics 8 (15): 4016-4032; Woodruff, R. S., and B. A. Sullenger, 2015 Arterioscler. Thromb. Vas. Biol. 35 (10): 2083-2091; and McConnell et al., 2020 Front. Chem. 8:434).

Hemophilia B (HB) is an X-linked blood coagulation disorder caused by Factor IX (FIX) deficiency. With a global incidence of 1 in 30000 born males, HB is considered a rare genetic disease. Severe HB patients with FIX activity of less than 1% comprise about 30% of the total FIX-deficient population. These patients exhibit recurrent spontaneous bleeding. Up to the mid-1980s, life expectancy of hemophilic patients was less than 12 years. Current conventional HB therapy is premised on prophylactic protein replacement. This therapeutic approach requires intravenous injections of recombinant FIX protein. The frequency of this treatment is dictated by the properties of the injected FIX protein. Specifically, short lived FIX protein requires 1-2 injections per week, while more advanced FIX proteins provides therapeutic levels of FIX following weekly or even bi-weekly infusions (Ward, P. and C. E. Walsh, 2016 Expert Rev. Hematol. 9 (7): 649-659). It is estimated that the lifetime cost of treating a moderate-to-severe hemophilia patient in the US ranges between $20-$23 million (Li et al. 2021 J. Med. Econ. 24 (1): 363-372).

Gene therapy protocols for HB are based on a single gene delivery intervention, which provides lifelong exogenous functional Factor IX cDNA as a replacement for mutated endogenous Factor IX genes. The first therapeutic gene delivery of Factor IX by AAV vectors was reported in 2011 (Ward and Walsh, 2016). Since then, several gene therapy clinical trials have been initiated (Perrin et al. 2019 Blood 133 (5): 407-414).

There are several weaknesses in the current gene and protein replacement therapies, including low efficacy of AAV vectors, which necessitates infusion of high dose vector particles; and immune responses to exogenous Factor IX, vector particles, and/or vector transduced cells (Ward and Walsh, 2016; Perrin 2019).

HB animal models are the basis of all studies aimed at advancing gene delivery and protein replacement protocols. Current animal models are premised on a small number of genetically Factor IX knocked-out (KO) mouse strains, and two colonies of naturally occurring Factor IX-deficient dogs. The small number of Factor IX-deficient mouse strains and the lack of a non-human primate model of HB are major impediments in studying the effects of the host genetic background on the outcome of gene and protein replacement therapies and limit the ability to predict the development of adverse effects in human patients, respectively.

Thus, in one embodiment, the present invention provides an in vitro method of modeling protein and/or gene replacement therapy in a protein non-deficient subject, comprising: (i) providing a sample from one or more protein non-deficient subject comprising an endogenous target protein; (ii) contacting the sample with an effective amount of an aptamer, wherein the effective amount of the aptamer reduces activity of the endogenous target protein as compared to uninhibited endogenous target protein activity; and (iii) contacting the sample with an effective amount of an exogenous aptamer-resistant replacement protein. In some embodiments, contacting the sample with an effective amount of an exogenous aptamer-resistant replacement protein may comprise contacting with an effective amount of a nucleic acid molecule (e.g., an mRNA), vector, cell, and/or composition of the invention encoding and/or comprising the aptamer-resistant replacement protein.

In another embodiment, the present invention provides an in vitro method of identifying genetic background effects of a protein non-deficient subject on an exogenous protein, comprising the steps of: (i) providing a sample from one or more protein non-deficient subject comprising an endogenous target protein; (ii) contacting the sample with an effective amount of an aptamer, wherein the effective amount of the aptamer reduces activity of the endogenous target protein as compared to uninhibited endogenous target protein activity; (iii) contacting the sample with an effective amount of an exogenous aptamer-resistant replacement protein; (iv) assaying for the presence of protein activity in the sample comprising the exogenous aptamer-resistant replacement protein and in a control (a sample from a subject of a different genetic background and contacted with the exogenous protein (e.g., a control/reference genetic background)); (v) comparing the relative level and/or presence of protein activity of the sample comprising the exogenous aptamer-resistant replacement protein and the control to identify genetic background effects of the subject on the efficacy and/or safety of the exogenous replacement protein; and (vi) associating the difference in relative level and/or presence of the protein activity with genetic background differences between the subject and the control. In some embodiments, contacting the sample with an effective amount of an exogenous aptamer-resistant replacement protein may comprise contacting with an effective amount of a nucleic acid molecule (e.g., an mRNA), vector, cell, and/or composition of the invention encoding and/or comprising the aptamer-resistant replacement protein.

The term “subject” as used herein is intended to mean any animal, in particular mammals, such as humans, and may, where appropriate, be used interchangeably with the term “patient,” e.g., a subject in need thereof. Suitable subjects include both avians and mammals. The term “avian” as used herein includes, but is not limited to, chickens, ducks, geese, quail, turkeys, pheasant, parrots, parakeets. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for a disorder including those described herein or that would benefit from the delivery of a recombinant aptamer-resistant protein including those described herein. For example, in particular embodiments, the subject has (or has had) or is at risk for a bleeding disorder. As a further option, the subject can be a laboratory animal and/or an animal model of disease.

As used herein, the term “protein non-deficient subject” refers to a subject of the present invention comprising an endogenous copy of the replacement protein, e.g., the subject may be replacement protein non-deficient. In the example of hemophilia and Factor IX treatment, a subject may comprise an endogenous Factor IX, and/or may be a non-hemophilic subject.

In some embodiments, the one or more protein non-deficient subject may be two or more subjects of different genetic backgrounds (e.g., Collaborative cross mouse strains as publicly available through csbio.unc.edu/CCstatus).

In some embodiments, the subject may be a subject selected by the methods of the present invention.

In some embodiments, the subject may be an aptamer-sensitive mammal. In some embodiments, the aptamer-resistant mammal is a canine, guinea pig, human, non-human primate (e.g., simian), mouse, or pig.

In some embodiments, a control may be a reference genetic background e.g., a known genetic background. For example, if the subject is a mouse, a control may be a known and/or well characterized laboratory mouse strain such as the C57BL/6, Balb/c, SJL, C3H/HeJ, or A/J mouse strain, or any other known and/or well characterized laboratory mouse strain.

In some embodiments, the associating step may comprise associating the difference(s) in relative level and/or presence of the protein activity with known genetic background differences between the subject and the control.

An effective amount of the aptamer may comprise an amount that reduces activity of the target protein by a phenotypically effective amount, e.g., an amount which results in a phenotypic outcome change such as, for the example of Factor IX, a resultant loss of blood clotting activity. In some embodiments, an effective amount of the aptamer may reduce activity of the endogenous target protein by at least 50, 60, 70, 80, 90, 91, 92, 93, 94, 95, 96, 97, 98, 99, or 100% of uninhibited endogenous target protein activity.

In some embodiments, the sample from the subject is a blood sample, a plasma sample, or any combination thereof. In some embodiments, the sample (e.g., the one or more sample(s)) may be an ex vivo or in vitro sample (e.g., a serum sample, e.g., a blood sample) of the subject.

In some embodiments, providing a sample from the one or more subject and contacting the aptamer to the sample may comprise performing a high-throughput screen of a plurality of samples, such as e.g., a sample from a plurality of subjects, e.g., a multi-sample in vitro plate-based assay.

In some embodiments, the present invention provides a method of modeling protein and/or gene replacement therapy in one or more protein non-deficient subject, comprising: (i) administering to the one or more subject an effective amount of an aptamer, wherein the effective amount of the aptamer reduces activity of an endogenous target protein as compared to uninhibited endogenous target protein activity (e.g., reduces activity by at least 50, 60, 70, 80, 90, 91, 92, 93, 94, 95, 96, 97, 98, 99, or 100% of uninhibited endogenous target protein activity); (ii) administering to the one or more subject an effective amount of an exogenous aptamer-resistant replacement protein. In some embodiments, administering an effective amount of an exogenous aptamer-resistant replacement protein may comprise administering an effective amount of a nucleic acid molecule (e.g., an mRNA), vector, cell, and/or composition of the invention encoding and/or comprising the aptamer-resistant replacement protein.

In some embodiments, the present invention provides a method of identifying genetic background effects on an exogenous protein in one or more protein non-deficient subject, comprising the steps of: (i) administering to the one or more subject an effective amount of an aptamer, wherein the effective amount of the aptamer reduces activity of an endogenous target protein as compared to uninhibited endogenous target protein activity (e.g., reduces activity of an endogenous target protein by at least 50, 60, 70, 80, 90, 91, 92, 93, 94, 95, 96, 97, 98, 99, or 100% of uninhibited endogenous target protein activity); (ii) administering to the one or more subject an effective amount of an exogenous aptamer-resistant replacement protein; (iii) assaying for the presence of protein activity in the one or more subject comprising the exogenous aptamer-resistant replacement protein and in a control (a subject of a different genetic background and administered the exogenous protein (e.g., a control/reference genetic background)); (iv) comparing the relative level and/or presence of protein activity of the one or more subject comprising the exogenous aptamer-resistant replacement protein and the control to identify genetic background effects of the subject on the efficacy and/or safety of the exogenous replacement protein; and (v) associating the difference in relative level and/or presence of the protein activity with genetic background differences between the one or more subject and the control. In some embodiments, administering an effective amount of an exogenous aptamer-resistant replacement protein may comprise administering an effective amount of a nucleic acid molecule (e.g., an mRNA), vector, cell, and/or composition of the invention encoding and/or comprising the aptamer-resistant replacement protein.

In some embodiments, the associating step may comprise a step of determining genetic background difference(s) between the subject and the control, and then associating the difference(s) in relative level and/or presence of the protein activity with determined genetic background difference(s) between the subject and the control.

In some embodiments, contacting with an effective amount of an exogenous replacement protein may comprise acquiring a plurality of samples of said subject and contacting the plurality of samples with a plurality of recombinant and/or exogenous replacement proteins, each sample of said plurality of samples being contacted with one of said plurality of recombinant and/or exogenous replacement proteins. A replacement protein and/or a plurality of replacement proteins of the present invention may comprise replacement proteins comprising modifications including but not limited to mutations, substitutions, deletions, insertions, post-translational modifications, etc., such as for example the Factor IX Padua mutation (“R338L”), codon-optimization, a 2xFc fusion Factor IX, and/or any other modified protein or other gene product to be screened. The source(s) of recombinant replacement proteins could be any naturally occurring gene, gene product, and/or protein, as well as entirely synthetically generated genes, gene products, and/or proteins. Replacement proteins may be engineered to be and/or identified as aptamer-resistant for example, through the methods as disclosed herein. In some embodiments, contacting with an effective amount of an exogenous replacement protein may comprise contacting with an effective amount of a nucleic acid molecule (e.g., an mRNA), vector, cell, and/or composition of the invention encoding and/or comprising the aptamer-resistant replacement protein.

In some embodiments, the methods disclosed herein may further comprise determining a safety profile of the exogenous replacement protein (e.g., the plurality of recombinant and/or exogenous replacement proteins, e.g., replacement proteins comprising modifications (mutations, substitutions, deletions, insertions, post-translational modifications, etc.) to be screened). In some embodiments, determining a safety profile may comprise screening for biological responses to the administered exogenous replacement protein, such as but not limited to an antibody response raised against the exogenous replacement protein, a cellular immune response raised against the exogenous replacement protein, and/or development of thrombo-emboli in response to the exogenous replacement protein, as well as any other standard safety testing known in the art which may desirous to perform, such as for F.D.A. and/or European Medicines Agency (E.M.A.) therapeutic drug approval procedures.

In some embodiments, further comprising determining efficacy of the administered exogenous replacement protein (e.g., the plurality of recombinant and/or exogenous replacement proteins, e.g., replacement proteins comprising modifications (mutations, substitutions, deletions, insertions, post-translational modifications, etc.) to be screened). In some embodiments, determining efficacy may comprise screening for biological effects of the administered exogenous replacement protein, such as but not limited to concentration over time of the exogenous replacement protein (e.g., Factor IX concentration and half-life), functional activity of the exogenous replacement protein (e.g., Factor IX activity), etc.

In some embodiments, administering to the subject may comprise acquiring a sample from the subject (e.g., a blood sample, a plasma sample, etc.), and contacting the aptamer to the sample in vitro.

In some embodiments, acquiring a sample from the subject and contacting the aptamer to the sample in vitro may comprise performing a high-throughput screen of a plurality of samples (e.g., a multi-sample in vitro plate-based assay).

In some embodiments, the present invention provides a method of selecting an exogenous replacement protein for modeling protein and/or gene replacement therapy in a protein non-deficient subject (e.g., an exogenous replacement protein of the present invention), comprising (i) introducing an amount of an aptamer to one or more sample(s) comprising an endogenous target protein of said aptamer, and then assaying for the presence of activity in the one or more sample(s) comprising said amount of said aptamer as compared to a control sample (e.g., a sample not comprising said aptamer), wherein the presence of activity in the one or more sample(s) comprising said aptamer indicates resistance of the endogenous target protein to inhibition by the aptamer introduced at said amount and wherein the lack of activity in the one or more sample(s) comprising the aptamer indicates sensitivity of the endogenous target protein to inhibition by the aptamer at said amount, as compared to a control sample; (ii) determining an effective amount of the aptamer for reducing activity of the endogenous target protein by repeating step (i) with an escalating amount of the aptamer until: (a) an escalating amount of the aptamer reduces activity of the endogenous target protein in the one or more sample(s) as compared to uninhibited endogenous target protein activity (e.g., reduces activity of the endogenous target protein by at least 50, 60, 70, 80, 90, 91, 92, 93, 94, 95, 96, 97, 98, 99, or 100% of uninhibited endogenous target protein activity), thereby determining the effective amount of the aptamer for the one or more sample, or (b) no escalating amount of the aptamer reduces activity of the endogenous target protein in the one or more sample(s) (e.g., by at least 50, 60, 70, 80, 90, 91, 92, 93, 94, 95, 96, 97, 98, 99, or 100% of uninhibited endogenous target protein activity); (iii) identifying the endogenous target protein comprised in the one or more sample(s) comprising an effective amount of said aptamer as sensitive to said aptamer (i.e., an aptamer-sensitive endogenous target protein), or identifying the endogenous target protein comprised in the one or more sample(s) unable to determine an effective amount of said aptamer as resistant to said aptamer (i.e., an aptamer-resistant endogenous target protein); and (iv) selecting the aptamer-resistant endogenous target protein as an exogenous (aptamer-resistant) replacement protein (e.g., for use in a method of the present invention).

In some embodiments, the present invention provides a method of selecting a model subject for modeling protein and/or gene replacement therapy in a protein non-deficient subject, comprising (i) introducing an amount of an aptamer to one or more sample(s) comprising an endogenous target protein of said aptamer, and then assaying for the presence of activity in the one or more sample(s) comprising said amount of said aptamer as compared to a control sample (e.g., a sample not comprising said aptamer), wherein the presence of activity in the one or more sample(s) comprising said aptamer indicates resistance of the endogenous target protein to inhibition by the aptamer introduced at said amount and wherein the lack of activity in the one or more sample(s) comprising the aptamer indicates sensitivity of the endogenous target protein to inhibition by the aptamer at said amount, as compared to a control sample; (ii) determining an effective amount of the aptamer for reducing activity of the endogenous target protein by repeating step (i) with an escalating amount of the aptamer until: (a) an escalating amount of the aptamer reduces activity of the endogenous target protein in the one or more sample(s) (e.g., reduces activity of the endogenous target protein in the one or more sample(s) by at least 50, 60, 70, 80, 90, 91, 92, 93, 94, 95, 96, 97, 98, 99, or 100% of uninhibited endogenous target protein activity, thereby determining the effective amount of the aptamer for the one or more sample, or (b) no escalating amount of the aptamer reduces activity of the endogenous target protein in the one or more sample(s) (e.g., by at least 50, 60, 70, 80, 90, 91, 92, 93, 94, 95, 96, 97, 98, 99, or 100% of uninhibited endogenous target protein activity); (iii) identifying the endogenous target protein comprised in the one or more sample(s) comprising an effective amount of said aptamer as sensitive to said aptamer (i.e., an aptamer-sensitive endogenous target protein), or identifying the endogenous target protein comprised in the one or more sample(s) unable to determine an effective amount of said aptamer as resistant to said aptamer (i.e., an aptamer-resistant endogenous target protein); and (iv) selecting the one or more sample(s) comprising the aptamer-sensitive endogenous target protein as a model subject for modeling protein and/or gene replacement therapy in a protein non-deficient subject (e.g., for use in a method of the present invention).

In some embodiments, the present invention provides a method of selecting an exogenous replacement protein for modeling protein replacement therapy in a protein non-deficient subject (e.g., the exogenous replacement protein of the present invention), comprising (i) introducing an effective amount of an aptamer to one or more sample(s) comprising an endogenous target protein of said aptamer, and then assaying for the presence of activity of the endogenous target protein in the one or more sample(s) comprising said amount of said aptamer, wherein the presence of activity in the one or more sample(s) comprising said aptamer as compared to a control sample that lacks activity (e.g., a sample not comprising said aptamer) indicates resistance of the endogenous target protein to inhibition by the aptamer and wherein the lack of activity in the one or more sample(s) comprising the aptamer indicates sensitivity of the endogenous target protein to inhibition by the aptamer; (ii) identifying the endogenous target protein comprised in the one or more sample(s) comprising an effective amount of said aptamer as sensitive to said aptamer (i.e., an aptamer-sensitive endogenous target protein), or identifying the endogenous target protein comprised in the one or more sample(s) unable to determine an effective amount of said aptamer as resistant to said aptamer (i.e., an aptamer-resistant endogenous target protein); and (iii) selecting the aptamer-resistant endogenous target protein as an exogenous (aptamer-resistant) replacement protein (e.g., for use in a method of the present invention).

In some embodiments, the present invention provides a method of selecting a model subject for modeling protein and/or gene replacement therapy in a protein non-deficient subject, comprising (i) introducing an effective amount of an aptamer to one or more sample(s) comprising an endogenous target protein of said aptamer, and then assaying for the presence of activity of the endogenous target protein in the one or more sample(s) comprising said amount of said aptamer, wherein the presence of activity in the one or more sample(s) comprising said aptamer as compared to a control sample that lacks activity (e.g., a sample not comprising said aptamer) indicates resistance of the endogenous target protein to inhibition by the aptamer and wherein the lack of activity in the one or more sample(s) comprising the aptamer indicates sensitivity of the endogenous target protein to inhibition by the aptamer; (ii) identifying the endogenous target protein comprised in the one or more sample(s) comprising an effective amount of said aptamer as sensitive to said aptamer (i.e., an aptamer-sensitive endogenous target protein), or identifying the endogenous target protein comprised in the one or more sample(s) unable to determine an effective amount of said aptamer as resistant to said aptamer (i.e., an aptamer-resistant endogenous target protein); and (iii) selecting the one or more sample(s) comprising the aptamer-sensitive endogenous target protein as a model subject for modeling protein and/or gene replacement therapy in a protein non-deficient subject (e.g., for use in a method of the present invention).

In some embodiments, a method of the present invention may further comprise the step of determining an effective amount of an aptamer for reducing activity of the endogenous target protein by (i) introducing a first amount of the aptamer to the one or more sample(s) comprising an endogenous target protein of said aptamer, and assaying for the presence of activity of the endogenous target protein in the one or more sample(s) comprising said first amount of said aptamer, wherein the presence of activity in the one or more sample(s) comprising said aptamer as compared to a control sample that lacks activity (e.g., a sample not comprising said aptamer) indicates resistance of the endogenous target protein to inhibition by the aptamer and wherein the lack of activity in the one or more sample(s) comprising the aptamer indicates sensitivity of the endogenous target protein to inhibition by the aptamer; and then (ii) repeating step (i) with an escalating amount of the aptamer until: (a) an escalating amount of the aptamer reduces activity of the endogenous target protein in the one or more sample(s) (e.g., by at least 50, 60, 70, 80, 90, 91, 92, 93, 94, 95, 96, 97, 98, 99, or 100% of uninhibited endogenous target protein activity), thereby determining the effective amount of the aptamer for the one or more sample, or (b) no escalating amount of the aptamer reduces activity of the endogenous target protein in the one or more sample(s) (e.g., by at least 50, 60, 70, 80, 90, 91, 92, 93, 94, 95, 96, 97, 98, 99, or 100% of uninhibited endogenous target protein activity).

The aptamer and the endogenous protein target of the aptamer of the invention may be any known aptamer and target protein pair known in the art or later discovered.

In some embodiments, the endogenous protein target of the aptamer may comprise Factor IX, von Willebrand Factor (VWF), activated protein C (APC), Factor I (fibrinogen), Factor VII, Factor XI, thrombin, kallikrein, Factor X, Factor II, or any combination thereof.

In some embodiments, the aptamer of the present invention may be selected from the group consisting of a Factor IX targeting aptamer (e.g., aptamer 9.3t); a von Willebrand Factor (VWF) targeting aptamer (e.g., ARC-1779 and/or BT200); a Factor XIa targeting aptamer (e.g., aptamer 12.7, or/and 11.16); an activated protein C (APC) targeting aptamer (e.g., HSO2); a Factor I (fibrinogen) targeting aptamer (e.g., HDI and/or ATW0007); a Factor IIa (thrombin) targeting aptamer (e.g., R9d14t, HD1 and/or HD22); a Factor II (prothrombin) targeting aptamer (e.g., R9d14t); a kallikrein targeting aptamer (e.g., Kalli-T4), a Factor Xa targeting aptamer (e.g., 11F7t or/and 10S-8); a Factor VII targeting aptamer (e.g., 7S-1); and any combination thereof.

The protein and/or gene replacement therapy of the present invention may model any protein deficiency disorder wherein an aptamer targeting said deficient protein is known or later developed. In some embodiments, the protein and/or gene replacement therapy may model a disorder selected from the group consisting of Factor IX deficiency (Hemophilia B), Factor VIII deficiency (Hemophilia A) Factor XI deficiency (Hemophilia C), von Willebrand disease, prekallikrein/kallikrein deficiency, Factor X deficiency, prothrombin deficiency, fibrinogen deficiency, Factor VII deficiency, and any combination thereof.

In some embodiments, the exogenous (aptamer-resistant) replacement protein may be an exogenous replacement protein selected by the methods of the present invention.

Additionally provided herein is an isolated nucleic acid molecule, comprising, consisting essentially of, and/or consisting of a nucleotide sequence encoding an aptamer-resistant exogenous replacement protein of the present invention. Such nucleic acids can be present in a vector, such as an expression cassette or a lentiviral vector. Thus, further embodiments of the invention are directed to expression cassettes designed to express a nucleotide sequence encoding any of the recombinant aptamer-resistant proteins of this invention. The nucleic acid molecules, cassettes, and/or constructs, as well as the vectors of this invention can be present in a cell (e.g., a transformed cell). Thus, various embodiments of the invention are directed to cells containing the vector (e.g., expression cassette). Such a cell can be isolated and/or present in a transgenic animal. Therefore, certain embodiments of the invention are further directed to a transgenic animal comprising a nucleic acid molecule comprising a nucleotide sequence encoding any of the recombinant aptamer-resistant proteins of the present invention.

In some embodiments, the nucleic acid molecule of this invention can have a coding sequence that has been optimized relative to a wild type coding sequence (e.g., a coding sequence for Factor IX) according to protocols well known in the art to, e.g., minimize usage of rare codons (e.g., human codons), remove alternative reading frames, etc., as would be known in the art (e.g., as described in PCT/US2007/071553, the disclosure of which is incorporated herein by reference in its entirety). An optimized nucleic acid molecule of this invention can also be optimized according to known protocols for example, to enhance the activity of a promoter, poly A signal, terminal repeats and/or other elements, as well as to modulate the activity and/or function of cis elements and trans elements involved in gene expression, regulation and/or production, etc., as would be well known in the art.

### Factor IX Compositions and Methods.

Thus, in some embodiments, the present invention provides a recombinant Factor IX protein of a mammal, comprising: (a) one or more substitution(s) of an XIa cleavage site-α and/or an XIa cleavage site-β of a human Factor IX protein; and/or (b) an N-terminal substitution of a human Factor IX N-terminus amino acid segment; wherein the recombinant Factor IX protein is resistant to inhibition by aptamer 9.3t.

In some embodiments, the recombinant Factor IX protein of a mammal may be of a rabbit or a hamster.

In some embodiments, a recombinant Factor IX protein of the present invention may further comprise substitution of one or more (e.g., 1, 2, 3, 4, 5, or more) neighboring amino acid residues of the human XIa cleavage site-α and/or the human XIa cleavage site-β.

In some embodiments, the substitution of a human Factor IX N-terminus amino acid segment may comprise, consist essentially of, or consist of at least 100 N-terminal amino acid residues of said human Factor IX protein, e.g., at least 100, 105, 110, 115, 120, 125, 130, 135, 140, 145, 150, 155, 160, 165, 170, 175, 176, 177, 178, 179, 180, 181, 182, 183, 184, or 185 or more N-terminal amino acid residues. For example, in some embodiments, the substitution of a human Factor IX N-terminus amino acid segment may comprise at least 100, at least 125, at least 150, at least 161, at least 179, or at least 180 N-terminal amino acid residues, or any value or a range therein, e.g., about 100 to about 180 N-terminal amino acid residues.

In some embodiments, the recombinant Factor IX protein may further comprise a leucine substitution at position 385 (e.g., R385L) wherein the numbering corresponds to SEQ ID NO: 2. This substitution is known in the art and may also be referred to as the amino acid substitution “R338L” or the “Padua” mutation (wherein the numbering corresponds to SEQ ID NO: 1, as related to complete sequence GenBank® accession number AAA56822.1 (with R384) and/or QWM97845.1 (with 384L), such as described in Simioni et al. 2009 N. Engl. J. Med. 361 (17): 1671-1675, the disclosures of which are incorporated herein by reference in their entirety).

Human Factor IX (lacking initial 46 residues
comprising signal peptide and protein
peptide; used for Padua “R338L” numbering)
SEQ ID NO: 1
YNSGKLEEFVOGNLERECMEEKCSFEEAREVFENTERTTEFWKQY
VDGDQCESNPCLNGGSCKDDINSYECWCPFGFEGKNCELDVTCNI
KNGRCEQFCKNSADNKVVCSCTEGYRLAENQKSCEPAVPFPCGRV
SVSQTSKLTRAEAVFPDVDYVNSTEAETILDNITQSTQSENDFTR
VVGGEDAKPGQFPWQVVLNGKVDAFCGGSIVNEKWIVTAAHCVET
GVKITVVAGEHNIEETEHTEQKRNVIRIIPHHNYNAAINKYNHDI
ALLELDEPLVLNSYVTPICIADKEYTNIFLKFGSGYVSGWGRVFH
KGRSALVLQYLRVPLVDRATCLRSTKFTIYNNMFCAGFHEGGRDS
CQGDSGGPHVTEVEGTSFLTGIISWGEECAMKGKYGIYTKVSRYV
NWIKEKTKLT.
WT rabbit Factor IX (does not contain Padua
substitution; wt “R” in place of Padua
highlighted; position 385); NP_001182654.1
SEQ ID NO: 2
MQFLNTIMAESPGLITICLLGYLLSAECTVELDHENATKILNRAK
RYNSGKLEEFVSGNLERECIEERCSFEEAREVFENTEKTTEFWKQ
YVDGDOCESNPCLNGGSCKDDINAYECWCQYGFEGKNCELDSTCS
IKNGRCEQFCRKNRNNKIICSCTEGYRLAENQKSCEPAVPFPCGR
VSVSHASKKITRATTIFSNTEYENFTEAETIRGNVTQDAQSSDDF
TRIVGGENAKPGQFPWQVLLNGKVEAFCGGSIINEKWVVTAAHCI
KPDDNITVVAGEYNIQETENTEQKRNVIRIIPYHKYNATINKYNH
DIALLELDKPLTLNSYVTPICIANREYTNIFLNFGSGYVSGWGRV
ENRGROASILQYLRVPFVDRATCLRSTKFTIYNNMFCAGFDVGGK
DSCEGDSGGPHVTEVEGTSFLTGIISWGEECAIKGKYGVYTRVSW
YVNWIKEKTKLT.

In some embodiments, the substitution of an XIa cleavage site-α may comprise a substitution of amino acid residue positions 182-201 and/or an insertion at the C-terminus (e.g., amino acid residue positions 463-481) corresponding to SEQ ID NO:2.

In some embodiments, the substitution of an XIa cleavage site-α may comprises, consist essentially of, or consist of the amino acid sequence SVSQTSKLTRAETVFPDVD (SEQ ID NO:7) or a sequence at least about 70% identical thereto, e.g., at least about 70, 75, 80, 85, 90, 91, 92, 93, 94, 95, 96, 97, 98, or 99% identical thereto.

In some embodiments, the substitution of an XIa cleavage site-β may comprise a substitution of amino acid residue positions 212-233 corresponding to SEQ ID NO:2.

In some embodiments, the substitution of an XIa cleavage site-β may comprise, consist essentially of, or consist of the amino acid sequence LDNITQSTQSFNDFTRVVGGED (SEQ ID NO:8) or a sequence at least about 70% identical thereto, e.g., at least about 70, 75, 80, 85, 90, 91, 92, 93, 94, 95, 96, 97, 98, or 99% identical thereto.

In some embodiments, a recombinant Factor IX protein of the present invention may further comprise one or more molecular tag (e.g., one or more Myc-tag).

In some embodiments, a recombinant Factor IX protein of the present invention may further comprise one or more (e.g., two or more, three or more, four or more, etc.) antibody fragments. Antibody fragments included within the scope of the present invention include, for example, Fab, F(ab′)2, and Fc fragments, and the corresponding fragments obtained from antibodies other than IgG. Such fragments can be produced by known techniques. For example, F(ab′) 2 fragments can be produced by pepsin digestion of the antibody molecule, and Fab fragments can be generated by reducing the disulfide bridges of the F (ab) 2 fragments. Alternatively, Fab expression libraries can be constructed to allow rapid and easy identification of monoclonal Fab fragments with the desired specificity (Huse et al., (1989) Science 254:1275-1281). In some embodiments, the antibody fragment may be an Fc fragment. In some embodiments, a recombinant Factor IX protein of the present invention may further comprise one or more (e.g., two or more) Fc fragments, optionally linked via a linker (e.g., a cleavable linker, e.g., an XIa cleavable linker).

In some embodiments, a recombinant Factor IX protein of the present invention may, comprise, consist essentially of, or consist of the amino acid sequence of SEQ ID NO:12 or an amino acid sequence at least 70% identical thereto, e.g., at least about 70, 75, 80, 85, 90, 91, 92, 93, 94, 95, 96, 97, 98, or 99% identical thereto.

Modified Rabbit Padua FIX comprising the human
activation peptide and cleavage sites α and β,
additional site α and myc tag at end, and
Padua substitution (position 384).
SEQ ID NO: 12
MQFLNTIMAESPGLITICLLGYLLSAECTVFLDHENATKILNRAK
RYNSGKLEEFVSGNLERECIEERCSFEEAREVFENTEKTTEFWKQ
YVDGDQCESNPCLNGGSCKDDINAYECWCQYGFEGKNCELDSTCS
IKNGRCEQFCRKNRNNKIICSCTEGYRLAENQKSCEPAVPFPCGR
VSVSQTSKLTRAETVFPDVDYENFTEAETILDNITQSTQSFNDFT
RVVGGEDAKPGQFPWQVLLNGKVEAFCGGSIINEKWVVTAAHCIK
PDDNITVVAGEYNIQETENTEQKRNVIRIIPYHKYNATINKYNHD
IALLELDKPLTLNSYVTPICIANREYTNIFLNFGSGYVSGWGRVF
NRGRQASILQYLRVPFVDRATCLLSTKFTIYNNMFCAGFDVGGKD
SCEGDSGGPHVTEVEGTSFLTGIISWGEECAIKGKYGVYTRVSWY
VNWIKEKTKLTSVSQTSKLTRAETVFPDVDGSEQKLISEEDLEQK
LISEEDLEQKLISEEDLEQKLISEEDL.

In some embodiments, a recombinant Factor IX protein of the present invention may, comprise, consist essentially of, or consist of the amino acid sequence of SEQ ID NO: 13 or an amino acid sequence at least 70% identical thereto, e.g., at least about 70, 75, 80, 85, 90, 91, 92, 93, 94, 95, 96, 97, 98, or 99% identical thereto.

Modified Rabbit Padua FIX comprising human
cleavage site α, additional human cleavage
site α and myc tag at end, and Padua
substitution (position 384);
sequence encoded by cDNA c1904.
SEQ ID NO: 13
MQFLNTIMAESPGLITICLLGYLLSAECTVFLDHENATKILNRAK
RYNSGKLEEFVSGNLERECIEERCSFEEAREVFENTEKTTEFWKQ
YVDGDQCESNPCLNGGSCKDDINAYECWCQYGFEGKNCELDSTCS
IKNGRCEQFCRKNRNNKIICSCTEGYRLAENQKSCEPAVPFPCGR
VSVSQTSKLTRAETVFPDVDYENFTEAETIRGNVTQDAQSSDDFT
RIVGGENAKPGQFPWQVLLNGKVEAFCGGSIINEKWVVTAAHCIK
PDDNITVVAGEYNIQETENTEQKRNVIRIIPYHKYNATINKYNHD
IALLELDKPLTLNSYVTPICIANREYTNIFLNFGSGYVSGWGRVF
NRGRQASILQYLRVPFVDRATCLLSTKFTIYNNMFCAGFDVGGKD
SCEGDSGGPHVTEVEGTSFLTGIISWGEECAIKGKYGVYTRVSWY
VNWIKEKTKLTSVSQTSKLTRAETVFPDVDGSEQKLISEEDLEQK
LISEEDLEQKLISEEDLEQKLISEEDL.

In some embodiments, a recombinant Factor IX protein of the present invention may, comprise, consist essentially of, or consist of the amino acid sequence of SEQ ID NO: 14 or an amino acid sequence at least 70% identical thereto, e.g., at least about 70, 75, 80, 85, 90, 91, 92, 93, 94, 95, 96, 97, 98, or 99% identical thereto.

Modified Rabbit Padua FIX comprising human
cleavage site β and additional human cleavage
site α and myc tag at end, and Padua
substitution (position 385).
SEQ ID NO: 14
MQFLNTIMAESPGLITICLLGYLLSAECTVFLDHENATKILNRAK
RYNSGKLEEFVSGNLERECIEERCSFEEAREVFENTEKTTEFWKQ
YVDGDQCESNPCLNGGSCKDDINAYECWCQYGFEGKNCELDSTCS
IKNGRCEQFCRKNRNNKIICSCTEGYRLAENQKSCEPAVPFPCGR
VSVSHASKKITRATTIFSNTEYENFTEAETILDNITQSTQSFNDF
TRVVGGEDAKPGQFPWQVLLNGKVEAFCGGSIINEKWVVTAAHCI
KPDDNITVVAGEYNIQETENTEQKRNVIRIIPYHKYNATINKYNH
DIALLELDKPLTLNSYVTPICIANREYTNIFLNFGSGYVSGWGRV
FNRGRQASILQYLRVPFVDRATCLLSTKFTIYNNMFCAGFDVGGK
DSCEGDSGGPHVTEVEGTSFLTGIISWGEECAIKGKYGVYTRVSW
YVNWIKEKTKLTSVSQTSKLTRAETVFPDVDGSEQKLISEEDLEQ
KLISEEDLEQKLISEEDLEQKLISEEDL.

In some embodiments, a recombinant Factor IX protein of the present invention may, comprise, consist essentially of, or consist of the amino acid sequence of SEQ ID NO: 15 or an amino acid sequence at least 70% identical thereto, e.g., at least about 70, 75, 80, 85, 90, 91, 92, 93, 94, 95, 96, 97, 98, or 99% identical thereto.

Modified Rabbit Padua FIX comprising additional
human cleavage site α and myc tag at end.
(Padua at 385) encoded by cDNA c1847)
SEQ ID NO: 15
MQFLNTIMAESPGLITICLLGYLLSAECTVFLDHENATKILNRAK
RYNSGKLEEFVSGNLERECIEERCSFEEAREVFENTEKTTEFWKQ
YVDGDQCESNPCLNGGSCKDDINAYECWCQYGFEGKNCELDSTCS
IKNGRCEQFCRKNRNNKIICSCTEGYRLAENQKSCEPAVPFPCGR
VSVSHASKKITRATTIFSNTEYENFTEAETIRGNVTQDAQSSDDF
TRIVGGENAKPGQFPWQVLLNGKVEAFCGGSIINEKWVVTAAHCI
KPDDNITVVAGEYNIQETENTEQKRNVIRIIPYHKYNATINKYNH
DIALLELDKPLTLNSYVTPICIANREYTNIFLNFGSGYVSGWGRV
FNRGRQASILQYLRVPFVDRATCLLSTKFTIYNNMFCAGFDVGGK
DSCEGDSGGPHVTEVEGTSFLTGIISWGEECAIKGKYGVYTRVSW
YVNWIKEKTKLTSVSQTSKLTRAETVFPDVDGSEQKLISEEDLEQ
KLISEEDLEQKLISEEDLEQKLISEEDL.

In some embodiments, a recombinant Factor IX protein of the present invention may, comprise, consist essentially of, or consist of the amino acid sequence of SEQ ID NO:16 or an amino acid sequence at least 70% identical thereto, e.g., at least about 70, 75, 80, 85, 90, 91, 92, 93, 94, 95, 96, 97, 98, or 99% identical thereto.

Chimeric Human/Rabbit Padua FIX comprising
179 N-terminal human amino acids and Padua
at 385, encoded by cDNA c1835
SEQ ID NO: 16
MQRVNMIMAESPGLITICLLGYLLSAECTVFLDHENANKILNRPK
RYNSGKLEEFVQGNLERECMEEKCSFEEAREVFENTERTTEFWKQ
YVDGDQCESNPCLNGGSCKDDINSYECWCPFGFEGKNCELDVTCN
IKNGRCEQFCKNSADNKVVCSCTEGYRLAENQKSCEPAVPFPCGG
VSVSHASKKITRATTIFSNTEYENFTEAETIRGNVTQRSQSSDDF
TRIVGGENAKPGQFPWQVLLNGKVEAFCGGSIINEKWVVTAAHCI
KPDDNITVVAGEYNIQETENTEQKRNVIRIIPYHKYNATINKYNH
DIALLELDKPLTLNSYVTPICIANREYTNIFLNFGSGYVSGWGRV
FNRGRQASILQYLRVPFVDRATCLLSTKFTIYNNMFCAGFDVGGK
DSCEGDSGGPHVTEVEGTSFLTGIISWGEECAIKGKYGVYTRVSW
YVNWIKEKTKLT.

In some embodiments, a recombinant Factor IX protein of the present invention may, comprise, consist essentially of, or consist of the amino acid sequence of SEQ ID NO:27 or an amino acid sequence at least 70% identical thereto, e.g., at least about 70, 75, 80, 85, 90, 91, 92, 93, 94, 95, 96, 97, 98, or 99% identical thereto.

AA sequence for FIX portion of novel molecule
linked to FC, encoded by cDNA cTK2294
SEQ ID NO: 27
MQFLNTIMAESPGLITICLLGYLLSAECTVFLDHENATKILNRAK
RYNSGKLEEFVSGNLERECIEERCSFEEAREVFENTEKTTEFWKQ
YVDGDQCESNPCLNGGSCKDDINAYECWCQYGFEGKNCELDSTCS
IKNGRCEQFCRKNRNNKIICSCTEGYRLAENQKSCEPAVPFPCGR
VSVSQTSKLTRAETVFPDVDYENFTEAETIRGNVTQDAQSSDDFT
RIVGGENAKPGQFPWQVLLNGKVEAFCGGSIINEKWVVTAAHCIK
PDDNITVVAGEYNIQETENTEQKRNVIRIIPYHKYNATINKYNHD
IALLELDKPLTLNSYVTPICIANREYTNIFLNFGSGYVSGWGRVF
NRGRQASILQYLRVPFVDRATCLLSTKFTIYNNMFCAGFDVGGKD
SCEGDSGGPHVTEVEGTSFLTGIISWGEECAIKGKYGVYTRVSWY
VNWIKEKTKLTSVSQTSKLTRAETVFPDVDGSEQKLISEEDLEQK
LISEEDLEQKLISEEDLEQKLISEEDLGSDKTHTCPPCPAPELLG
GPSVFLFPPKPKDTLMISRTPEVTCVVVDVSHEDPEVKFNWYVDG
VEVHNAKTKPREEQYNSTYRVVSVLTVLHQDWLNGKEYKCKVSNK
ALPAPIEKTISKAKGQPREPQVYTLPPSRDELTKNQVSLTCLVKG
FYPSRLAVEWESNGQPENNYKTTPPVLDSDGSFFLYSKLTVDKSR
WQQGNVFSCSVMHEALHNHYTQKSLSLSPGKGGGSGGGSGGGSDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSRDE
LTKNQVSLTCLVKGFYPSRLAVEWESNGQPENNYKTTPPVLDSDG
SFFLYSKLTVDKSRWQQGNVFSCSVMHEALHNHYTQKSLSLSPGK.

The recombinant Factor IX proteins according to the invention may be produced and characterized by methods well known in the art and as described herein. These methods include determination of clotting time (partial thromboplastin time (PPT) assay) and administration of the Factor IX protein to a test animal to determine recovery, half-life, and bioavailability by an appropriate immunoassay and/or activity-assay, as are well known in the art.

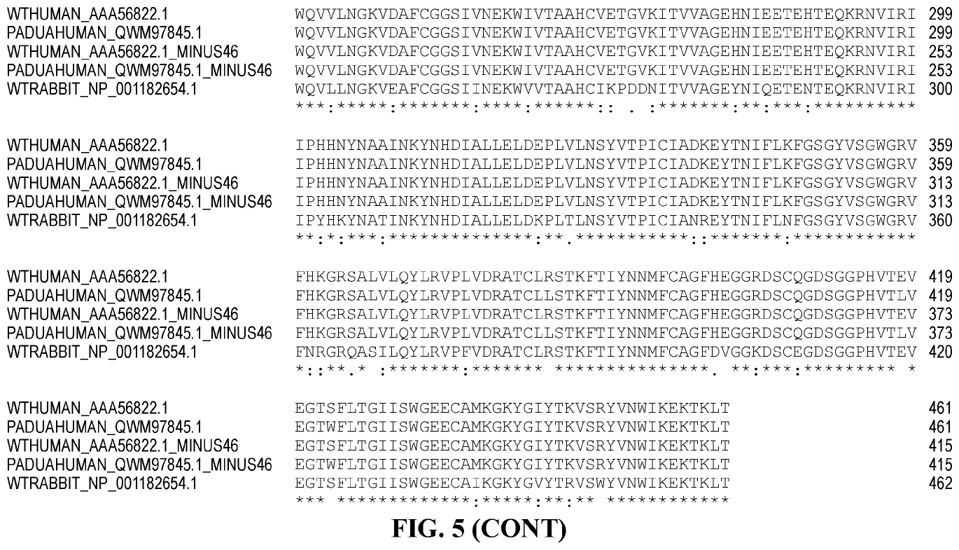

It would be understood that the modifications described herein and the recombinant Factor IX proteins described above provide five examples of how the amino acid sequences above can be obtained and that, due to the degeneracy of the amino acid codons, numerous other modifications can be made to a nucleotide sequence encoding the recombinant Factor IX protein to obtain these amino acid sequences. In addition, amino acid and/or nucleic acid sequences for species and/or strain homologs of proteins of the present invention may have differences in residues, position, and numbering. Accordingly, the amino acid residue numbering provided in the amino acid sequences set forth here is based on the reference amino acid sequence provided, e.g., WT Rabbit Factor IX (SEQ ID NO:2). However it would be readily understood by one of ordinary skill in the art that the equivalent amino acid positions in other Factor IX amino acid sequences can be readily identified and employed in the production of the recombinant proteins of this invention. Those skilled in the art, upon review of the present disclosure, will be familiar with numerous methods of aligning sequences to identify correlate residues, positions, and/or numbering as needed in related proteins. For example, FIG. 5 provides a sequence alignment between WT rabbit Factor IX protein sequence and WT human Factor IX protein sequences.

Thus, in some embodiments, the present invention provides an isolated nucleic acid molecule, comprising, consisting essentially of, and/or consisting of a nucleotide sequence encoding a recombinant Factor IX protein of the present invention. Such nucleic acids can be present in a vector, such as an expression cassette or a lentiviral vector. Thus, further embodiments of the invention are directed to expression cassettes designed to express a nucleotide sequence encoding any of the recombinant Factor IX proteins of this invention.

A nucleic acid molecule of this invention can be present in a vector, which can be a plasmid vector or a viral vector. Nonlimiting examples of a viral vector of this invention include a retroviral vector, a lentiviral vector, an adenoviral vector, an adeno-associated viral vector, an alphavirus vector, a vaccinia viral vector, a herpesviral vector, etc., as are known in the art.

Furthermore, a vector of this invention can have a vector genome that has been optimized relative to a wildtype vector genome, e.g., to enhance the activity of viral cis elements required for replication, packaging and/or delivery, etc., as would be well known in the art. Such an optimized vector can comprise an optimized transcription cassette, optimized terminal repeats, etc., as would be well known in the art.

The nucleic acid molecules, cassettes, and/or constructs, as well as the vectors of this invention can be present in a cell (e.g., a transformed cell). Thus, various embodiments of the invention are directed to cells containing the vector (e.g., expression cassette). In various embodiments, the nucleic acid molecule of this invention can be present in a cell transiently and/or can be stably integrated into the genome of the cell and/or the genome of the cell. The nucleotide sequence can also be stably expressed in the cell even without being integrated into the genome, via a plasmid or other nucleic acid construct as would be well known in the art. Such a cell can be isolated and/or present in a transgenic animal. Therefore, certain embodiments of the invention are further directed to a transgenic animal comprising a nucleic acid molecule comprising a nucleotide sequence encoding any of the recombinant Factor IX proteins of the present invention.

In some embodiments, the nucleic acid molecule of this invention can have a coding sequence that has been optimized relative to a wild type coding sequence (e.g., a coding sequence for Factor IX) according to protocols well known in the art to, e.g., minimize usage of rare codons (e.g., human codons), remove alternative reading frames, etc., as would be known in the art (e.g., as described in PCT/US2007/071553, the disclosure of which is incorporated herein by reference in its entirety). An optimized nucleic acid molecule of this invention can also be optimized according to known protocols for example, to enhance the activity of a promoter, poly A signal, terminal repeats and/or other elements, as well as to modulate the activity and/or function of cis elements and trans elements involved in gene expression, regulation and/or production, etc., as would be well known in the art.

Thus, in some embodiments, the present invention provides an isolated nucleic acid molecule comprising, consisting essentially of, or consisting of the nucleotide sequence of SEQ ID NOs: 17-23 or 26, or a nucleic acid sequence at least 70% identical thereto, e.g., at least about 70, 75, 80, 85, 90, 91, 92, 93, 94, 95, 96, 97, 98, or 99% identical thereto.

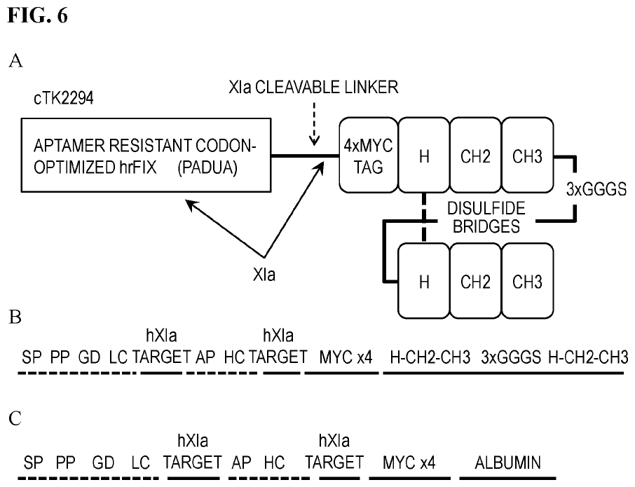

pTK1335 human FIX Padua cDNA
SEQ ID NO: 17
atgcagcgcgtgaacatgatcatggccgagagccctggcctgatc
accatctgcctgctgggctacctgctgagcgccgagtgcaccgtg
ttcctggaccacgagaacgccaacaagatcctgaaccggcccaag
agatacaacagcggcaagctggaggagttcgtgcagggcaacctg
gagagggagtgcatggaggagaagtgcagcttcgaggaggccagg
gaagtgttcgagaacaccgageggaccaccgagttctggaagcag
tacgtggacggcgaccagtgcgagagcaacccttgcctgaacggc
ggcagctgcaaggacgacatcaacagctacgagtgctggtgccct
ttcggcttcgagggcaagaactgcgagctggacgtgacctgcaac
atcaagaacggccgctgcgagcagttctgcaagaacagcgccgac
aacaaagtggtgtgtagctgcaccgagggctacagactggccgag
aaccagaagagctgcgagcccgccgtgcccttcccctgcggcaga
gtgagcgtgtcccagaccagcaagctgaccagagccgagaccgtg
ttccccgacgtggactacgtgaatagcaccgaggccgagaccatc
ctggacaacatcacccagagcacccagtccttcaacgacttcacc
agagttgtgggcggcgaggacgccaagcccggccagttcccctgg
caggtggtgctgaacggcaaagtggatgccttctgcggcggcagc
atcgtgaacgagaagtggatcgtgacagccgcccactgcgtggag
accggcgtgaagatcaccgtggtggccggcgaacacaatatcgag
gagaccgagcacaccgagcagaagcggaacgtcatccggattatc
ccccaccacaactacaacgccgccatcaacaagtacaaccacgac
atcgccctgctggagctggacgagcctctggtgctgaatagctac
gtgacccccatctgcategccgacaaggagtacaccaacatcttc
ctgaagttcggcagcggctacgtgtccggctggggcagagtgttc
cacaagggcagaagcgccctggtgctgcagtacctgagagtgccc
ctggtggacagagccacctgcctgttgagcaccaagttcaccatc
tacaacaacatgttctgcgccggcttccacgagggcggcagagac
agctgccagggcgacagcggcggaccccacgtgaccgaagtggag
ggcaccagcttcctgaccggcatcatcagctggggcgaggagtgc
gccatgaagggcaagtacggcatctacaccaaagtgagccggtac
gtgaactggatcaaggagaaaaccaagctgacctga.
pTK1835 Chimeric human/rabbit Padua Factor
IX Padua cDNA
SEQ ID NO: 18
ATGCAGCGCGTGAACATGATTATGGCCGAGAGCCCTGGCCTGATC
ACCATCTGCCTGCTGGGCTACCTGCTGAGCGCCGAGTGTACCGTG
TTCCTGGACCACGAGAACGCCAACAAGATCCTGAACAGACCCAAG
AGATACAACAGCGGCAAGCTGGAAGAGTTCGTGCAGGGCAACCTG
GAACGCGAGTGCATGGAAGAGAAGTGCAGCTTCGAAGAGGCCAGA
GAGGTGTTCGAGAACACCGAGAGAACCACCGAGTTCTGGAAGCAG
TACGTGGACGGCGACCAGTGCGAGAGCAACCCTTGTCTGAACGGC
GGCAGCTGCAAGGACGACATCAACAGCTACGAGTGCTGGTGCCCC
TTCGGCTTCGAGGGCAAGAACTGCGAGCTGGACGTGACCTGCAAC
ATCAAGAACGGCAGATGCGAGCAGTTCTGCAAGAACAGCGCCGAC
AACAAGGTCGTGTGCTCCTGCACCGAGGGCTACAGACTGGCCGAG
AACCAGAAGTCCTGCGAGCCCGCTGTGCCCTTTCCTTGTGGCGGC
GTGTCCGTGTCTCACGCCAGCAAGAAGATCACCAGAGCCACCACC
ATCTTCAGCAACACCGAGTACGAGAACTTCACCGAGGCCGAGACA
ATCAGAGGCAACGTGACCCAGAGAAGCCAGAGCAGCGACGACTTC
ACCAGAATCGTGGGCGGCGAGAACGCTAAGCCCGGCCAGTTCCCG
TGGCAGGTGCTGCTGAACGGAAAGGTGGAAGCCTTCTGCGGCGGC
TCCATCATCAACGAGAAATGGGTCGTGACAGCCGCCCACTGCATC
AAGCCCGACGACAACATCACCGTGGTGGCTGGCGAGTACAACATC
CAGGAAACCGAGAATACCGAACAGAAACGGAACGTGATCCGCATC
ATCCCCTACCACAAGTACAACGCCACAATCAACAAGTATAACCAC
GATATCGCCCTGCTGGAACTGGACAAGCCCCTGACCCTGAACTCC
TACGTGACCCCCATCTGTATCGCCAACAGAGAGTACACCAACATC
TTTCTGAACTTCGGCAGCGGCTACGTGTCCGGCTGGGGCAGAGTG
TTCAACAGAGGCAGACAGGCCAGCATCCTGCAGTACCTGAGAGTG
CCCTTCGTGGACAGGGCCACCTGTCTGCTGAGCACCAAGTTCACC
ATCTACAACAACATGTTCTGCGCTGGCTTCGATGTGGGCGGCAAG
GACTCTTGCGAGGGCGATTCTGGCGGCCCTCACGTGACAGAGGTG
GAAGGCACCAGCTTTCTGACCGGCATCATCAGCTGGGGCGAGGAA
TGCGCCATCAAGGGCAAATACGGCGTGTACACCAGAGTGTCTTGG
TACGTGAACTGGATCAAAGAAAAGACCAAGCTGACCTGA.
pTK1847 Rabbit FIX Padua cDNA
SEQ ID NO: 19
ATGCAGTTTCTGAACACCATCATGGCCGAGAGCCCTGGCCTGATC
ACCATCTGCCTGCTGGGCTACCTGCTGAGCGCCGAGTGTACCGTG
TTCCTGGACCACGAGAACGCCACAAAGATCCTGAACAGAGCCAAG
AGATACAACAGCGGCAAGCTGGAAGAGTTCGTGTCCGGCAACCTG
GAAAGAGAGTGCATCGAGGAAAGATGCAGCTTCGAAGAGGCCAGA
GAGGTGTTCGAGAACACCGAAAAGACCACCGAGTTCTGGAAGCAG
TACGTGGACGGCGACCAGTGCGAGAGCAACCCTTGTCTGAACGGC
GGCAGCTGCAAGGACGACATCAACGCCTACGAGTGCTGGTGCCAG
TACGGCTTCGAGGGCAAGAACTGCGAGCTGGACAGCACCTGTAGC
ATCAAGAACGGCAGATGCGAGCAGTTCTGCCGGAAGAACAGAAAC
AACAAGATCATCTGCAGCTGTACCGAGGGCTACAGACTGGCCGAG
AACCAGAAGTCCTGCGAGCCCGCTGTGCCTTTCCCATGCGGAAGA
GTGTCCGTGTCTCACGCCAGCAAGAAGATCACCAGAGCCACCACC
ATCTTCAGCAACACCGAGTACGAGAACTTCACCGAGGCCGAGACA
ATCAGAGGCAACGTGACCCAGGACGCCCAGAGCAGCGACGACTTC
ACCAGAATCGTGGGCGGCGAGAACGCTAAGCCCGGCCAGTTCCCG
TGGCAGGTGCTGCTGAACGGAAAGGTGGAAGCCTTCTGCGGCGGC
TCCATCATCAACGAGAAATGGGTCGTGACAGCCGCCCACTGCATC
AAGCCCGACGACAACATCACAGTGGTGGCCGGCGAGTACAACATC
CAGGAAACCGAGAACACAGAGCAGAAACGGAACGTGATCAGGATC
ATCCCCTACCACAAGTACAACGCTACCATCAACAAGTATAACCAC
GATATCGCCCTGCTGGAACTGGACAAGCCCCTGACCCTGAACAGC
TACGTGACCCCCATCTGTATCGCCAACAGAGAGTACACCAACATC
TTTCTGAACTTCGGCAGCGGCTACGTGTCCGGATGGGGCAGAGTG
TTCAACAGAGGCAGACAGGCCAGCATCCTGCAGTACCTGAGAGTG
CCCTTCGTGGACAGGGCCACCTGTCTGCTGAGCACCAAGTTCACC
ATCTACAACAACATGTTCTGCGCCGGCTTCGATGTGGGCGGCAAG
GATTCTTGCGAGGGCGATTCTGGCGGCCCTCACGTGACAGAGGTG
GAAGGCACCAGCTTTCTGACCGGCATCATCAGCTGGGGCGAGGAA
TGCGCCATCAAGGGCAAATACGGCGTGTACACCAGAGTGTCTTGG
TACGTGAACTGGATCAAAGAAAAGACAAAGCTGACCAGCGTGTCC
CAGACCAGCAAGCTGACAAGAGCCGAGACAGTGTTCCCCGATGTG
GACGGCTCCGAGCAGAAGCTGATCTCCGAAGAGGACCTGGAACAG
AAACTGATCAGCGAGGAAGATCTGGAACAGAAGCTGATTTCTGAA
GAAGATCTGGAACAGAAACTGATCTCCGAAGAGGATCTCTGA.
pTK1904 Rabbit FIX with human Cleavage site
α Padua
SEQ ID NO: 20
ATGCAGTTTCTGAACACCATCATGGCCGAGAGCCCTGGCCTGATC
ACCATCTGCCTGCTGGGCTACCTGCTGAGCGCCGAGTGTACCGTG
TTCCTGGACCACGAGAACGCCACAAAGATCCTGAACAGAGCCAAG
AGATACAACAGCGGCAAGCTGGAAGAGTTCGTGTCCGGCAACCTG
GAAAGAGAGTGCATCGAGGAAAGATGCAGCTTCGAAGAGGCCAGA
GAGGTGTTCGAGAACACCGAAAAGACCACCGAGTTCTGGAAGCAG
TACGTGGACGGCGACCAGTGCGAGAGCAACCCTTGTCTGAACGGC
GGCAGCTGCAAGGACGACATCAACGCCTACGAGTGCTGGTGCCAG
TACGGCTTCGAGGGCAAGAACTGCGAGCTGGACAGCACCTGTAGC
ATCAAGAACGGCAGATGCGAGCAGTTCTGCCGGAAGAACAGAAaC
AACAAGATCATCTGCAGCTGTACCGAGGGCTACAGACTGGCCGAG
AACCAGAAGTCCTGCGAgCCCGCTGTGCCTTTCCCATGCGGAAGA
GTGTCCGTGTCACAAACTTCTAAGCTCACCCGTGCTGAGACTGTT
TTTCCTGATGTGGACTACGAGAACTTCACCGAGGCCGAGACAATC
AGAGGCAACGTGACCCAGGACGCCCAGAGCAGCGACGACTTCACC
AGAATCGTGGGCGGCGAGAACGCTAAGCCCGGCCAGTTCCCGTGG
CAGGTGCTGCTGAACGGAAAGGTGGAAGCCTTCTGCGGCGGCTCC
ATCATCAACGAGAAATGGGTCGTGACAGCCGCCCACTGCATCAAG
CCCGACGACAACATCACAGTGGTGGCCGGCGAGTACAACATCCAG
GAAACCGAGAACACAGAGCAGAAACGGAACGTGATCAGGATCATC
CCCTACCACAAGTACAACGCTACCATCAACAAGTATAACCACGAT
ATCGCCCTGCTGGAACTGGACAAGCCCCTGACCCTGAACAGCTAC
GTGACCCCCATCTGTATCGCCAACAGAGAGTACACCAACATCTTT
CTGAACTTCGGCAGCGGCTACGTGTCCGGATGGGGCAGAGTGTTC
AACAGAGGCAGACAGGCCAGCATCCTGCAGTACCTGAGAGTGCCC
TTCGTGGACAGGGCCACCTGTCTGCTGAGCACCAAGTTCACCATC
TACAACAACATGTTCTGCGCCGGCTTCGATGTGGGCGGCAAGGAT
TCTTGCGAGGGCGATTCTGGCGGCCCTCACGTGACAGAGGTGGAA
GGCACCAGCTTTCTGACCGGCATCATCAGCTGGGGCGAGGAATGC
GCCATCAAGGGCAAATACGGCGTGTACACCAGAGTGTCTTGGTAC
GTGAACTGGATCAAAGAAAAGACAAAGCTGACCAGCGTGTCCCAG
ACCAGCAAGCTGACAAGAGCCGAGACAGTGTTCCCCGATGTGGAC
GGCTCCGAGCAGAAGCTGATCTCCGAAGAGGACCTGGAACAGAAA
CTGATCAGCGAGGAAGATCTGGAACAGAAGCTGATTTCTGAAGAA
GATCTGGAACAGAAACTGATCTCCGAAGAGGATCTCTGA.
Rabbit FIX with human Cleavage site β Padua
SEQ ID NO: 21
ATGCAGTTTCTGAACACCATCATGGCCGAGAGCCCTGGCCTGATC
ACCATCTGCCTGCTGGGCTACCTGCTGAGCGCCGAGTGTACCGTG
TTCCTGGACCACGAGAACGCCACAAAGATCCTGAACAGAGCCAAG
AGATACAACAGCGGCAAGCTGGAAGAGTTCGTGTCCGGCAACCTG
GAAAGAGAGTGCATCGAGGAAAGATGCAGCTTCGAAGAGGCCAGA
GAGGTGTTCGAGAACACCGAAAAGACCACCGAGTTCTGGAAGCAG
TACGTGGACGGCGACCAGTGCGAGAGCAACCCTTGTCTGAACGGC
GGCAGCTGCAAGGACGACATCAACGCCTACGAGTGCTGGTGCCAG
TACGGCTTCGAGGGCAAGAACTGCGAGCTGGACAGCACCTGTAGC
ATCAAGAACGGCAGATGCGAGCAGTTCTGCCGGAAGAACAGAAAC
AACAAGATCATCTGCAGCTGTACCGAGGGCTACAGACTGGCCGAG
AACCAGAAGTCCTGCGAGCCCGCTGTGCCTTTCCCATGCGGAAGA
GTGTCCGTGTCTCACGCCAGCAAGAAGATCACCAGAGCCACCACC
ATCTTCAGCAACACCGAGTACGAGAACTTCACCGAGGCCGAGACA
ATCctggacaacatcacccagagcacccagtccttcaacgacttc
accagagttgtgggcggcgaggacGCTAAGCCCGGCCAGTTCCCG
TGGCAGGTGCTGCTGAACGGAAAGGTGGAAGCCTTCTGCGGCGGC
TCCATCATCAACGAGAAATGGGTCGTGACAGCCGCCCACTGCATC
AAGCCCGACGACAACATCACAGTGGTGGCCGGCGAGTACAACATC
CAGGAAACCGAGAACACAGAGCAGAAACGGAACGTGATCAGGATC
ATCCCCTACCACAAGTACAACGCTACCATCAACAAGTATAACCAC
GATATCGCCCTGCTGGAACTGGACAAGCCCCTGACCCTGAACAGC
TACGTGACCCCCATCTGTATCGCCAACAGAGAGTACACCAACATC
TTTCTGAACTTCGGCAGCGGCTACGTGTCCGGATGGGGCAGAGTG
TTCAACAGAGGCAGACAGGCCAGCATCCTGCAGTACCTGAGAGTG
CCCTTCGTGGACAGGGCCACCTGTCTGCTGAGCACCAAGTTCACC
ATCTACAACAACATGTTCTGCGCCGGCTTCGATGTGGGCGGCAAG
GATTCTTGCGAGGGCGATTCTGGCGGCCCTCACGTGACAGAGGTG
GAAGGCACCAGCTTTCTGACCGGCATCATCAGCTGGGGCGAGGAA
TGCGCCATCAAGGGCAAATACGGCGTGTACACCAGAGTGTCTTGG
TACGTGAACTGGATCAAAGAAAAGACAAAGCTGACCAGCGTGTCC
CAGACCAGCAAGCTGACAAGAGCCGAGACAGTGTTCCCCGATGTG
GACGGCTCCGAGCAGAAGCTGATCTCCGAAGAGGACCTGGAACAG
AAACTGATCAGCGAGGAAGATCTGGAACAGAAGCTGATTTCTGAA
GAAGATCTGGAACAGAAACTGATCTCCGAAGAGGATCTCTGA.
Rabbit FIX Rabbit FIX with human Cleavage
site α and Cleavage site β Padua
SEQ ID NO: 22
ATGCAGTTTCTGAACACCATCATGGCCGAGAGCCCTGGCCTGATC
ACCATCTGCCTGCTGGGCTACCTGCTGAGCGCCGAGTGTACCGTG
TTCCTGGACCACGAGAACGCCACAAAGATCCTGAACAGAGCCAAG
AGATACAACAGCGGCAAGCTGGAAGAGTTCGTGTCCGGCAACCTG
GAAAGAGAGTGCATCGAGGAAAGATGCAGCTTCGAAGAGGCCAGA
GAGGTGTTCGAGAACACCGAAAAGACCACCGAGTTCTGGAAGCAG
TACGTGGACGGCGACCAGTGCGAGAGCAACCCTTGTCTGAACGGC
GGCAGCTGCAAGGACGACATCAACGCCTACGAGTGCTGGTGCCAG
TACGGCTTCGAGGGCAAGAACTGCGAGCTGGACAGCACCTGTAGC
ATCAAGAACGGCAGATGCGAGCAGTTCTGCCGGAAGAACAGAAAC
AACAAGATCATCTGCAGCTGTACCGAGGGCTACAGACTGGCCGAG
AACCAGAAGTCCTGCGAGCCCGCTGTGCCTTTCCCATGCGGAAGA
GTGTCCGTGTCACAAACTTCTAAGCTCACCCGTGCTGAGACTGTT
TTTCCTGATGTGGACTACGAGAACTTCACCGAGGCCGAGACAATC
ctggacaacatcacccagagcacccagtccttcaacgacttcacc
agagttgtgggcggcgaggacGCTAAGCCCGGCCAGTTCCCGTGG
CAGGTGCTGCTGAACGGAAAGGTGGAAGCCTTCTGCGGCGGCTCC
ATCATCAACGAGAAATGGGTCGTGACAGCCGCCCACTGCATCAAG
CCCGACGACAACATCACAGTGGTGGCCGGCGAGTACAACATCCAG
GAAACCGAGAACACAGAGCAGAAACGGAACGTGATCAGGATCATC
CCCTACCACAAGTACAACGCTACCATCAACAAGTATAACCACGAT
ATCGCCCTGCTGGAACTGGACAAGCCCCTGACCCTGAACAGCTAC
GTGACCCCCATCTGTATCGCCAACAGAGAGTACACCAACATCTTT
CTGAACTTCGGCAGCGGCTACGTGTCCGGATGGGGCAGAGTGTTC
AACAGAGGCAGACAGGCCAGCATCCTGCAGTACCTGAGAGTGCCC
TTCGTGGACAGGGCCACCTGTCTGCTGAGCACCAAGTTCACCATC
TACAACAACATGTTCTGCGCCGGCTTCGATGTGGGCGGCAAGGAT
TCTTGCGAGGGCGATTCTGGCGGCCCTCACGTGACAGAGGTGGAA
GGCACCAGCTTTCTGACCGGCATCATCAGCTGGGGCGAGGAATGC
GCCATCAAGGGCAAATACGGCGTGTACACCAGAGTGTCTTGGTAC
GTGAACTGGATCAAAGAAAAGACAAAGCTGACCAGCGTGTCCCAG
ACCAGCAAGCTGACAAGAGCCGAGACAGTGTTCCCCGATGTGGAC
GGCTCCGAGCAGAAGCTGATCTCCGAAGAGGACCTGGAACAGAAA
CTGATCAGCGAGGAAGATCTGGAACAGAAGCTGATTTCTGAAGAA
GATCTGGAACAGAAACTGATCTCCGAAGAGGATCTCTGA.
Rabbit FIX Rabbit FIX with human Cleavage
site α and Cleavage site β and
human active peptide Padua
SEQ ID NO: 23
ATGCAGTTTCTGAACACCATCATGGCCGAGAGCCCTGGCCTGATC
ACCATCTGCCTGCTGGGCTACCTGCTGAGCGCCGAGTGTACCGTG
TTCCTGGACCACGAGAACGCCACAAAGATCCTGAACAGAGCCAAG
AGATACAACAGCGGCAAGCTGGAAGAGTTCGTGTCCGGCAACCTG
GAAAGAGAGTGCATCGAGGAAAGATGCAGCTTCGAAGAGGCCAGA
GAGGTGTTCGAGAACACCGAAAAGACCACCGAGTTCTGGAAGCAG
TACGTGGACGGCGACCAGTGCGAGAGCAACCCTTGTCTGAACGGC
GGCAGCTGCAAGGACGACATCAACGCCTACGAGTGCTGGTGCCAG
TACGGCTTCGAGGGCAAGAACTGCGAGCTGGACAGCACCTGTAGC
ATCAAGAACGGCAGATGCGAGCAGTTCTGCCGGAAGAACAGAAAC
AACAAGATCATCTGCAGCTGTACCGAGGGCTACAGACTGGCCGAG
AACCAGAAGTCCTGCGAGCCCGCTGTGCCTTTCCCATGCGGAAGA
GTGTCCGTGTCACAAACTTCTAAGCTCACCCGTGCTGAGACTGTT
TTTCCTGATGTGGACTACGtGAACagCACCGAGGCCGAGACAATC
ctggacaacatcacccagagcacccagtccttcaacgacttcacc
agagttgtgggcggcgaggacGCTAAGCCCGGCCAGTTCCCGTGG
CAGGTGCTGCTGAACGGAAAGGTGGAAGCCTTCTGCGGCGGCTCC
ATCATCAACGAGAAATGGGTCGTGACAGCCGCCCACTGCATCAAG
CCCGACGACAACATCACAGTGGTGGCCGGCGAGTACAACATCCAG
GAAACCGAGAACACAGAGCAGAAACGGAACGTGATCAGGATCATC
CCCTACCACAAGTACAACGCTACCATCAACAAGTATAACCACGAT
ATCGCCCTGCTGGAACTGGACAAGCCCCTGACCCTGAACAGCTAC
GTGACCCCCATCTGTATCGCCAACAGAGAGTACACCAACATCTTT
CTGAACTTCGGCAGCGGCTACGTGTCCGGATGGGGCAGAGTGTTC
AACAGAGGCAGACAGGCCAGCATCCTGCAGTACCTGAGAGTGCCC
TTCGTGGACAGGGCCACCTGTCTGCTGAGCACCAAGTTCACCATC
TACAACAACATGTTCTGCGCCGGCTTCGATGTGGGCGGCAAGGAT
TCTTGCGAGGGCGATTCTGGCGGCCCTCACGTGACAGAGGTGGAA
GGCACCAGCTTTCTGACCGGCATCATCAGCTGGGGCGAGGAATGC
GCCATCAAGGGCAAATACGGCGTGTACACCAGAGTGTCTTGGTAC
GTGAACTGGATCAAAGAAAAGACAAAGCTGACCAGCGTGTCCCAG
ACCAGCAAGCTGACAAGAGCCGAGACAGTGTTCCCCGATGTGGAC
GGCTCCGAGCAGAAGCTGATCTCCGAAGAGGACCTGGAACAGAAA
CTGATCAGCGAGGAAGATCTGGAACAGAAGCTGATTTCTGAAGAA
GATCTGGAACAGAAACTGATCTCCGAAGAGGATCTCTGA.
cTK2294 (depicted in FIG. 6) encoding a
fusion protein comprising aptamer resistant
factor IX and a tandem of Fc regions.
SEQ ID NO: 26
atgcagtttctgaacaccatcatggccgagagccctggcctgatc
accatctgcctgctgggctacctgctgagegccgagtgtaccgtg
ttcctggaccacgagaacgccacaaagatcctgaacagagccaag
agatacaacagcggcaagctggaagagttcgtgtccggcaacctg
gaaagagagtgcatcgaggaaagatgcagcttcgaagaggccaga
gaggtgttcgagaacaccgaaaagaccaccgagttctggaagcag
tacgtggacggcgaccagtgcgagagcaacccttgtctgaacggc
ggcagctgcaaggacgacatcaacgcctacgagtgctggtgccag
tacggcttcgagggcaagaactgcgagctggacagcacctgtagc
atcaagaacggcagatgcgagcagttctgccggaagaacagaaac
aacaagatcatctgcagctgtaccgagggctacagactggccgag
aaccagaagtcctgcgagcccgctgtgcctttcccatgcggaaga
gtgtccgtgtcacaaacttctaagctcacccgtgctgagactgtt
tttcctgatgtggactacgagaacttcaccgaggccgagacaatc
agaggcaacgtgacccaggacgcccagagcagcgacgacttcacc
agaatcgtgggcggcgagaacgctaagcccggccagttcccgtgg
caggtgctgctgaacggaaaggtggaagccttctgcggcggctcc
atcatcaacgagaaatgggtcgtgacagccgcccactgcatcaag
cccgacgacaacatcacagtggtggccggcgagtacaacatccag
gaaaccgagaacacagagcagaaacggaacgtgatcaggatcatc
ccctaccacaagtacaacgctaccatcaacaagtataaccacgat
atcgccctgctggaactggacaagcccctgaccctgaacagctac
gtgacccccatctgtatcgccaacagagagtacaccaacatcttt
ctgaacttcggcagcggctacgtgtccggatggggcagagtgttc
aacagaggcagacaggccagcatcctgcagtacctgagagtgccc
ttcgtggacagggccacctgtctgctgagcaccaagttcaccatc
tacaacaacatgttctgcgccggcttcgatgtgggcggcaaggat
tcttgcgagggcgattctggcggccctcacgtgacagaggtggaa
ggcaccagctttctgaccggcatcatcagctggggcgaggaatgc
gccatcaagggcaaatacggcgtgtacaccagagtgtcttggtac
gtgaactggatcaaagaaaagacaaagctgaccagcgtgtcccag
accagcaagctgacaagagccgagacagtgttccccgatgtggac
ggctccgagcagaagctgatctccgaagaggacctggaacagaaa
ctgatcagcgaggaagatctggaacagaagctgatttctgaagaa
gatctggaacagaaactgatctccgaagaggatctcGGCAGCGAC
AAGACCCACACCTGTCCTCCATGTCCCGCCCCTGAACTGCTGGGC
GGACCTAGCGTGTTCCTGTTCCCCCCAAAGCCCAAGGACACCCTG
ATGATCAGCCGGACCCCCGAAGTGACCTGCGTGGTGGTGGATGTG
TCCCACGAGGACCCTGAAGTGAAGTTCAATTGGTATGTGGATGGC
GTGGAAGTGCACAACGCCAAGACAAAGCCCAGAGAGGAACAGTAC
AACTCCACCTACCGGGTGGTGTCCGTGCTGACCGTGCTGCACCAG
GACTGGCTGAATGGCAAAGAGTATAAGTGCAAAGTGTCCAACAAG
GCCCTGCCTGCCCCCATCGAGAAAACCATCAGCAAGGCCAAGGGC
CAGCCCCGCGAACCCCAGGTGTACACACTGCCCCCTAGCAGGGAC
GAGCTGACCAAGAACCAGGTGTCCCTGACCTGTCTCGTGAAGGGC
TTCTACCCTAGCCGGCTGGCCGTGGAATGGGAGAGCAATGGCCAG
CCCGAGAACAATTACAAGACCACCCCCCCTGTGCTGGACAGCGAC
GGCTCATTCTTCCTGTACAGCAAACTGACCGTGGACAAGAGCCGG
TGGCAGCAGGGCAATGTGTTCAGCTGTAGCGTGATGCACGAGGCC
CTGCACAACCACTACACCCAGAAGTCTCTGAGCCTGAGCCCCGGC
AAGGGCGGAGGAAGTGGGGGAGGATCTGGCGGCGGCTCCGATAAG
ACACATACCTGCCCCCCTTGCCCTGCCCCAGAGCTGCTGGGAGGC
CCTTCTGTGTTTCTGTTTCCACCTAAGCCTAAAGATACACTGATG
ATCTCCCGCACACCTGAAGTGACATGTGTGGTGGTGGACGTGTCA
CATGAAGATCCAGAAGTGAAGTTTAATTGGTACGTGGACGGGGTG
GAAGTGCATAATGCTAAGACCAAACCCCGGGAAGAACAGTATAAC
AGCACATACAGAGTGGTGTCTGTGCTGACAGTGCTGCATCAGGAT
TGGCTGAACGGGAAAGAATACAAATGTAAAGTGTCTAACAAAGCT
CTGCCCGCTCCTATCGAAAAGACAATCTCCAAGGCTAAAGGACAG
CCCAGAGAACCTCAGGTGTACACACTGCCTCCATCCCGCGACGAG
CTGACAAAAAATCAGGTGTCACTGACATGCCTCGTGAAGGGGTTT
TATCCATCTAGGCTGGCTGTGGAATGGGAATCCAACGGACAGCCT
GAAAACAACTATAAGACAACACCTCCCGTGCTGGACTCCGATGGC
TCATTTTTTCTGTATTCCAAGCTGACTGTGGATAAGTCCAGATGG
CAGCAGGGAAACGTGTTCTCCTGTTCTGTGATGCATGAAGCTCTG
CATAATCATTATACCCAGAAAAGCCTGTCCCTGTCCCCTGGCAAG
TGA.

Many expression vectors can be used to create genetically engineered cells. Some expression vectors are designed to express large quantities of recombinant proteins after amplification of transfected cells under a variety of conditions that favor selected, high expressing cells. Some expression vectors are designed to express large quantities of recombinant proteins without the need for amplification under selection pressure. The present invention includes the production of genetically engineered cells according to methods standard in the art and is not dependent on the use of any specific expression vector or expression system.

To create a genetically engineered cell to produce large quantities of a Factor IX protein of this invention, cells are transfected with an expression vector that contains the cDNA encoding a target protein (e.g., the recombinant Factor IX protein of this invention). In some embodiments, the target protein is expressed with selected co-transfected enzymes that cause proper post-translational modification of the target protein to occur in a given cell system.

The cell may be selected from a variety of sources, but is otherwise a cell that may be transfected with an expression vector containing a nucleic acid, preferably a cDNA encoding a recombinant Factor IX protein.

The practice of the present invention employs, unless otherwise indicated, conventional techniques of molecular biology, microbiology, recombinant DNA, and immunology, which are within the skill of the art. Such techniques are explained fully in the literature. See, e.g., Sambrook, et al., Molecular Cloning; A Laboratory Manual, 2nd ed. (1989); DNA Cloning, Vols. I and II (D. N Glover, ed. 1985); Oligonucleotide Synthesis (M. J. Gait, ed. 1984); Nucleic Acid Hybridization (B. D. Hames & S. J. Higgins, eds. 1984); Transcription and Translation (B. D. Hames & S. J. Higgins, eds. 1984); Animal Cell Culture (R. I. Freshney, ed. 1986); Immobilized Cells and Enzymes (IRL Press, 1986); B. Perbal, A Practical Guide to Molecular Cloning (1984); the series, Methods in Enzymology (Academic Press, Inc.), particularly Vols. 154 and 155 (Wu and Grossman, and Wu, eds., respectively); Gene Transfer Vectors for Mammalian Cells (J. H. Miller and M. P. Calos, eds. 1987, Cold Spring Harbor Laboratory); Immunochemical Methods in Cell and Molecular Biology, Mayer and Walker, eds. (Academic Press, London, 1987); Scopes, Protein Purification: Principles and Practice, 2nd ed. 1987 (Springer-Verlag, N.Y.); and Handbook of Experimental Immunology Vols I-IV (D. M. Weir and C. C. Blackwell, eds 1986). All patents, patent applications, and publications cited in the specification are incorporated herein by reference in their entireties.

The production of cloned genes, recombinant DNA, vectors, transformed host cells, proteins and protein fragments by genetic engineering is well known. See, e.g., U.S. Pat. No. 4,761,371 to Bell et al. at Col. 6, line 3 to Col. 9, line 65; U.S. Pat. No. 4,877,729 to Clark et al. at Col. 4, line 38 to Col. 7, line 6; U.S. Pat. No. 4,912,038 to Schilling at Col. 3, line 26 to Col. 14, line 12; and U.S. Pat. No. 4,879,224 to Wallner at Col. 6, line 8 to Col. 8, line 59.

A vector is a replicable DNA construct. Vectors are used herein either to amplify nucleic acid encoding Factor IX protein and/or to express nucleic acid which encodes Factor IX protein. An expression vector is a replicable nucleic acid construct in which a nucleotide sequence encoding a Factor IX protein is operably linked to suitable control sequences capable of effecting the expression of the nucleotide sequence to produce a Factor IX protein in a suitable host. The need for such control sequences will vary depending upon the host selected and the transformation method chosen. Generally, control sequences include a transcriptional promoter, an optional operator sequence to control transcription, a sequence encoding suitable mRNA ribosomal binding sites, and sequences that control the termination of transcription and translation.

Vectors comprise plasmids, viruses (e.g., adenovirus, cytomegalovirus, lentivirus), phage, and integratable DNA fragments (i.e., fragments integratable into the host genome by recombination). The vector replicates and functions independently of the host genome, or may, in some instances, integrate into the genome itself. Expression vectors can contain a promoter and RNA binding sites that are operably linked to the gene to be expressed and are operable in the host organism.

DNA regions or nucleotide sequences are operably linked or operably associated when they are functionally related to each other. For example, a promoter is operably linked to a coding sequence if it controls the transcription of the sequence; or a ribosome binding site is operably linked to a coding sequence if it is positioned so as to permit translation of the sequence.

Transformed host cells are cells which have been transformed, transduced and/or transfected with Factor IX protein vector(s) constructed using recombinant DNA techniques.

Suitable host cells include prokaryote, yeast or higher eukaryotic cells such as mammalian cells and insect cells. Cells derived from multicellular organisms are a particularly suitable host for recombinant Factor IX protein synthesis, and mammalian cells are particularly preferred. Propagation of such cells in cell culture has become a routine procedure (Tissue Culture, Academic Press, Kruse and Patterson, editors (1973)). Examples of useful host cell lines are VERO and HeLa cells, Chinese hamster ovary (CHO) cell lines, and WI138, HEK 293, BHK, COS-7, CV, and MDCK cell lines. Expression vectors for such cells ordinarily include (if necessary) an origin of replication, a promoter located upstream from the nucleotide sequence encoding Factor IX protein to be expressed and operatively associated therewith, along with a ribosome binding site, an RNA splice site (if intron-containing genomic DNA is used), a polyadenylation site, and a transcriptional termination sequence. In a preferred embodiment, expression is carried out in Chinese Hamster Ovary (CHO) cells using the expression system of U.S. Pat. No. 5,888,809, which is incorporated herein by reference in its entirety.

The transcriptional and translational control sequences in expression vectors to be used in transforming vertebrate cells are often provided by viral sources. For example, commonly used promoters are derived from polyoma, Adenovirus 2, and Simian Virus 40 (SV40). See. e.g., U.S. Pat. No. 4,599,308.

An origin of replication may be provided either by construction of the vector to include an exogenous origin, such as may be derived from SV 40 or other viral (e.g., polyoma, adenovirus, VSV, or BPV) source, or may be provided by the host cell chromosomal replication mechanism. If the vector is integrated into the host cell chromosome, the latter is often sufficient.

Rather than using vectors which contain viral origins of replication, one can transform mammalian cells by the method of cotransformation with a selectable marker and the nucleic acid encoding the Factor IX protein. Examples of suitable selectable markers are dihydrofolate reductase (DHFR) or thymidine kinase. This method is further described in U.S. Pat. No. 4,399,216 which is incorporated by reference herein in its entirety.

Other methods suitable for adaptation to the synthesis of Factor IX protein in recombinant vertebrate cell culture include those described in Gething et al. Nature 293:620 (1981); Mantei et al. Nature 281:40; and Levinson et al., EPO Application Nos. 117,060A and 117,058A, the entire contents of each of which are incorporated herein by reference.

Host cells such as insect cells (e.g., cultured Spodoptera frugiperda cells) and expression vectors such as the baculovirus expression vector (e.g., vectors derived from Autographa californica MNPV, Trichoplusia ni MNPV, Rachiplusia ou MNPV, or Galleria ou MNPV) may be employed in carrying out the present invention, as described in U.S. Pat. Nos. 4,745,051 and 4,879,236 to Smith et al. In general, a baculovirus expression vector comprises a baculovirus genome containing the nucleotide sequence to be expressed inserted into the polyhedrin gene at a position ranging from the polyhedrin transcriptional start signal to the ATG start site and under the transcriptional control of a baculovirus polyhedrin promoter.

Prokaryote host cells include gram negative or gram positive organisms, for example Escherichia coli (E. coli) or bacilli. Higher eukaryotic cells include established cell lines of mammalian origin as described below. Exemplary host cells are E. coli W3110 (ATCC 27,325), E. coli B, E. coli X1776 (ATCC 31,537) and E. coli 294 (ATCC 31,446). A broad variety of suitable prokaryotic and microbial vectors are available. E. coli is typically transformed using pBR322. Promoters most commonly used in recombinant microbial expression vectors include the betalactamase (penicillinase) and lactose promoter systems (Chang et al. Nature 275:615 (1978); and Goeddel et al. Nature 281:544 (1979)), a tryptophan (trp) promoter system (Goeddel et al. Nucleic Acids Res. 8:4057 (1980) and EPO App. Publ. No. 36,776) and the tac promoter (De Boer et al. Proc. Natl. Acad. Sci. USA 80:21 (1983)). The promoter and Shine-Dalgarno sequence (for prokaryotic host expression) are operably linked to the nucleic acid encoding the Factor IX protein, i.e., they are positioned so as to promote transcription of Factor IX messenger RNA from DNA.

Eukaryotic microbes such as yeast cultures may also be transformed with protein-encoding vectors (see, e.g., U.S. Pat. No. 4,745,057). Saccharomyces cerevisiae is the most commonly used among lower eukaryotic host microorganisms, although a number of other strains are commonly available. Yeast vectors may contain an origin of replication from the 2 micron yeast plasmid or an autonomously replicating sequence (ARS), a promoter, nucleic acid encoding Factor IX protein, sequences for polyadenylation and transcription termination, and a selection gene. An exemplary plasmid is YRp7, (Stinchcomb et al. Nature 282:39 (1979); Kingsman et al. Gene 7:141 (1979); Tschemper et al. Gene 10:157 (1980)). Suitable promoting sequences in yeast vectors include the promoters for metallothionein, 3-phosphoglycerate kinase (Hitzeman et al. J. Biol. Chem. 255:2073 (1980) or other glycolytic enzymes (Hess et al. J. Adv. Enzyme Reg. 7:149 (1968); and Holland et al. Biochemistry 17:4900 (1978)). Suitable vectors and promoters for use in yeast expression are further described in R. Hitzeman et al., EPO Publn. No. 73,657.

Cloned coding sequences of the present invention may encode FIX of any species of origin, including mouse, rat, dog, opossum, rabbit, cat, pig, horse, sheep, cow, guinea pig, platypus, and human, but preferably encode Factor IX protein of human origin. Nucleic acid encoding Factor IX that is hybridizable with nucleic acid encoding proteins disclosed herein is also encompassed. Hybridization of such sequences may be carried out under conditions of reduced stringency or even stringent conditions (e.g., stringent conditions as represented by a wash stringency of 0.3M NaCl, 0.03M sodium citrate, 0.1% SDS at 60° C. or even 70° C.) to nucleic acid encoding Factor IX protein disclosed herein in a standard in situ hybridization assay. See, e.g., Sambrook et al., Molecular Cloning, A Laboratory Manual (2d Ed. 1989)

### Cold Spring Harbor Laboratory).

The FIX proteins produced according to the invention may be expressed in transgenic animals by known methods. See for example, U.S. Pat. No. 6,344,596, which is incorporated herein by reference in its entirety. In brief, transgenic animals may include but are not limited to farm animals (e.g., pigs, goats, sheep, cows, horses, rabbits and the like) rodents (such as mice, rats and guinea pigs), and domestic pets (for example, cats and dogs). Livestock animals such as pigs, sheep, goats and cows, are particularly preferred in some embodiments.

The transgenic animal of this invention is produced by introducing into a single cell embryo an appropriate polynucleotide that encodes a human Factor IX protein of this invention in a manner such that the polynucleotide is stably integrated into the DNA of germ line cells of the mature animal, and is inherited in normal Mendelian fashion. The transgenic animal of this invention would have a phenotype of producing the FIX protein in body fluids and/or tissues. The FIX protein would be removed from these fluids and/or tissues and processed, for example for therapeutic use. (See, e.g., Clark et al. “Expression of human anti-hemophilic factor IX in the milk of transgenic sheep” Bio/Technology 7:487-492 (1989); Van Cott et al. “Haemophilic factors produced by transgenic livestock: abundance can enable alternative therapies worldwide” Haemophilia 10 (4): 70-77 (2004), the entire contents of which are incorporated by reference herein).

DNA molecules can be introduced into embryos by a variety of means including but not limited to microinjection, calcium phosphate mediated precipitation, liposome fusion, or retroviral infection of totipotent or pluripotent stem cells. The transformed cells can then be introduced into embryos and incorporated therein to form transgenic animals. Methods of making transgenic animals are described, for example, in Transgenic Animal Generation and Use by L. M. Houdebine, Harwood Academic Press, 1997. Transgenic animals also can be generated using methods of nuclear transfer or cloning using embryonic or adult cell lines as described for example in Campbell et al., Nature 380:64-66 (1996) and Wilmut et al., Nature 385:810-813 (1997). Further a technique utilizing cytoplasmic injection of DNA can be used as described in U.S. Pat. No. 5,523,222.

Factor IX-producing transgenic animals can be obtained by introducing a chimeric construct comprising Factor IX-encoding sequences. Methods for obtaining transgenic animals are well-known. See, for example, Hogan et al., MANIPULATING THE MOUSE EMBRYO, (Cold Spring Harbor Press 1986); Krimpenfort et al., Bio/Technology 9:88 (1991); Palmiter et al., Cell 41:343 (1985), Kraemer et al., GENETIC MANIPULATION OF THE EARLY MAMMALIAN EMBRYO, (Cold Spring Harbor Laboratory Press 1985); Hammer et al., Nature 315:680 (1985); Wagner et al., U.S. Pat. No. 5,175,385; Krimpenfort et al., U.S. Pat. No. 5,175,384, Janne et al., Ann. Med. 24:273 (1992), Brem et al., Chim. Oggi. 11:21 (1993), Clark et al., U.S. Pat. No. 5,476,995, all incorporated by reference herein in their entireties.

In some embodiments, cis-acting regulatory regions may be used that are “active” in mammary tissue in that the promoters are more active in mammary tissue than in other tissues under physiological conditions where milk is synthesized. Such promoters include but are not limited to the short and long whey acidic protein (WAP), short and long a, B and K casein, α-lactalbumin and β-lactoglobulin (“BLG”) promoters. Signal sequences can also be used in accordance with this invention that direct the secretion of expressed proteins into other body fluids, particularly blood and urine. Examples of such sequences include the signal peptides of secreted coagulation factors including signal peptides of Factor IX, protein C, and tissue-type plasminogen activator.

Among the useful sequences that regulate transcription, in addition to the promoters discussed above, are enhancers, splice signals, transcription termination signals, polyadenylation sites, buffering sequences, RNA processing sequences and other sequences which regulate the expression of transgenes.

Preferably, the expression system or construct includes a 3′ untranslated region downstream of the nucleotide sequence encoding the desired recombinant protein. This region can increase expression of the transgene. Among the 3′ untranslated regions useful in this regard are sequences that provide a poly A signal.

Suitable heterologous 3′-untranslated sequences can be derived, for example, from the SV40 small t antigen, the casein 3′ untranslated region, or other 3′ untranslated sequences well known in this art. Ribosome binding sites are also important in increasing the efficiency of expression of FIX. Likewise, sequences that regulate the post-translational modification of FIX are useful in the invention.

Factor IX coding sequences, along with vectors and host cells for the expression thereof, are disclosed in European Patent App. 373012, European Patent App. 251874, PCT Patent App. 8505376, PCT Patent App. 8505125, European Patent App. 162782, and PCT Patent App. 8400560, all of which are incorporated by reference herein in their entireties.

Pharmaceutical formulations comprising a recombinant replacement protein (e.g., recombinant Factor IX protein), nucleic acids, vectors, cells or compositions of the invention and a pharmaceutically acceptable carrier are also provided, and can be formulated for administration in a pharmaceutical carrier in accordance with known techniques. See, e.g., Remington, The Science And Practice of Pharmacy (latest edition). In the manufacture of a pharmaceutical composition according to embodiments of the present invention, the composition of the invention is typically admixed with, inter alia, a pharmaceutically acceptable carrier. By “pharmaceutically acceptable carrier” is meant a carrier that is compatible with other ingredients in the pharmaceutical composition and that is not harmful or deleterious to the subject. The carrier may be a solid or a liquid, or both, and is preferably formulated with the composition of the invention as a unit-dose formulation, for example, a tablet, which may contain from about 0.01 or 0.5% to about 95% or 99% by weight of the composition. The pharmaceutical compositions are prepared by any of the well-known techniques of pharmacy including, but not limited to, admixing the components, optionally including one or more accessory ingredients. In certain embodiments, the pharmaceutically acceptable carrier is sterile and would be deemed suitable for administration into human subjects according to regulatory guidelines for pharmaceutical compositions comprising the carrier.

Furthermore, a “pharmaceutically acceptable” component such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include any of the standard pharmaceutical carriers such as phosphate buffered saline solutions, water, emulsions such as oil/water emulsion, microemulsions and various types of wetting agents.

The pharmaceutical formulations of the invention can optionally comprise other medicinal agents, pharmaceutical agents, stabilizing agents, buffers, carriers, diluents, salts, tonicity adjusting agents, wetting agents, and the like, for example, sodium acetate, sodium lactate, sodium chloride, potassium chloride, calcium chloride, sorbitan monolaurate, triethanolamine oleate, etc.

For injection, the carrier will typically be a liquid. For other methods of administration, the carrier may be either solid or liquid. For inhalation administration, the carrier will be respirable, and is typically in a solid or liquid particulate form. The compositions of the invention can be formulated for administration in a pharmaceutical carrier in accordance with known techniques. See, e.g., Remington, The Science And Practice of Pharmacy (9th Ed. 1995). In the manufacture of a pharmaceutical composition according to the invention, the VLPs are typically admixed with, inter alia, an acceptable carrier. The carrier can be a solid or a liquid, or both, and is optionally formulated with the compound as a unit-dose formulation, for example, a tablet. A variety of pharmaceutically acceptable aqueous carriers can be used, e.g., water, buffered water, 0.9% saline, 0.3% glycine, hyaluronic acid, pyrogen-free water, pyrogen-free phosphate-buffered saline solution, bacteriostatic water, or Cremophor EL[R] (BASF, Parsippany, N.J.), and the like. These compositions can be sterilized by conventional techniques. The formulations of the invention can be prepared by any of the well-known techniques of pharmacy.

The pharmaceutical formulations can be packaged for use as is, or lyophilized, the lyophilized preparation generally being combined with a sterile aqueous solution prior to administration. The compositions can further be packaged in unit/dose or multi-dose containers, for example, in sealed ampoules and vials.

The pharmaceutical formulations can be formulated for administration by any method known in the art according to conventional techniques of pharmacy. For example, the compositions can be formulated to be administered intranasally, by inhalation (e.g., oral inhalation), orally, buccally (e.g., sublingually), rectally, vaginally, topically, intrathecally, intraocularly, transdermally, by parenteral administration (e.g., intramuscular [e.g., skeletal muscle], intravenous, subcutaneous, intradermal, intrapleural, intracerebral and intra-arterial, intrathecal), or topically (e.g., to both skin and mucosal surfaces, including airway surfaces). For intranasal or inhalation administration, the pharmaceutical formulation can be formulated as an aerosol (this term including both liquid and dry powder aerosols). For example, the pharmaceutical formulation can be provided in a finely divided form along with a surfactant and propellant. Typical percentages of the composition are 0.01-20% by weight, preferably 1-10%. The surfactant is generally nontoxic and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, preferably 0.25-5%. The balance of the composition is ordinarily propellant. A carrier can also be included, if desired, as with lecithin for intranasal delivery. Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles can likewise be produced with any solid particulate medicament aerosol generator, by techniques known in the pharmaceutical art. Intranasal administration can also be by droplet administration to a nasal surface. Injectable formulations can be prepared in conventional forms, either as liquid solutions or suspensions, solid forms suitable for solution or suspension in liquid prior to injection, or as emulsions. Alternatively, one can administer the pharmaceutical formulations in a local rather than systemic manner, for example, in a depot or sustained-release formulation. Extemporaneous injection solutions and suspensions can be prepared from sterile powders, granules and tablets of the kind previously described. For example, an injectable, stable, sterile formulation of the invention in a unit dosage form in a sealed container can be provided. The formulation can be provided in the form of a lyophilizate, which can be reconstituted with a suitable pharmaceutically acceptable carrier to form a liquid composition suitable for injection into a subject. The unit dosage form can be from about 1 μg to about 10 grams of the formulation. When the formulation is substantially water-insoluble, a sufficient amount of emulsifying agent, which is pharmaceutically acceptable, can be included in sufficient quantity to emulsify the formulation in an aqueous carrier. One such useful emulsifying agent is phosphatidyl choline.

Pharmaceutical formulations suitable for oral administration can be presented in discrete units, such as capsules, cachets, lozenges, or tables, as a powder or granules; as a solution or a suspension in an aqueous or non-aqueous liquid; or as an oil-in-water or water-in-oil emulsion. Oral delivery can be performed by complexing a compound(s) of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the protein(s) and a suitable carrier (which may contain one or more accessory ingredients as noted above). In general, the pharmaceutical formulations are prepared by uniformly and intimately admixing the compound(s) with a liquid or finely divided solid carrier, or both, and then, if necessary, shaping the resulting mixture. For example, a tablet can be prepared by compressing or molding a powder or granules, optionally with one or more accessory ingredients. Compressed tablets are prepared by compressing, in a suitable machine, the formulation in a free-flowing form, such as a powder or granules optionally mixed with a binder, lubricant, inert diluent, and/or surface active/dispersing agent(s). Molded tablets are made by molding, in a suitable machine, the powdered protein moistened with an inert liquid binder.

Pharmaceutical formulations suitable for buccal (sub-lingual) administration include lozenges comprising the compound(s) in a flavored base, usually sucrose and acacia or tragacanth; and pastilles in an inert base such as gelatin and glycerin or sucrose and acacia.

Pharmaceutical formulations suitable for parenteral administration can comprise sterile aqueous and non-aqueous injection solutions, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like.

Pharmaceutical formulations suitable for rectal administration are optionally presented as unit dose suppositories. These can be prepared by admixing the active agent with one or more conventional solid carriers, such as for example, cocoa butter and then shaping the resulting mixture.

Pharmaceutical formulations suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical formulation of the present invention with a lipophilic reagent (e.g., DMSO) that is capable of passing into the skin.

Pharmaceutical formulations suitable for transdermal administration can be in the form of discrete patches adapted to remain in intimate contact with the epidermis of the subject for a prolonged period of time. Formulations suitable for transdermal administration can also be delivered by iontophoresis (see, for example, Pharmaceutical Research 3:318 (1986)) and typically take the form of a buffered aqueous solution of the compound(s). Suitable formulations can comprise citrate or bis\tris buffer (pH 6) or ethanol/water and can contain from 0.1 to 0.2M active ingredient.

Further, the composition can be formulated as a liposomal formulation. The lipid layer employed can be of any conventional composition and can either contain cholesterol or can be cholesterol-free. The liposomes that are produced can be reduced in size, for example, through the use of standard sonication and homogenization techniques.

The liposomal formulations can be lyophilized to produce a lyophilizate which can be reconstituted with a pharmaceutically acceptable carrier, such as water, to regenerate a liposomal suspension.

Additionally provided herein are methods comprising the recombinant Factor IX proteins of the present invention.

Thus, one aspect of the invention comprises a method of identifying a Factor IX protein as resistant to inhibition by aptamer 9.3t, comprising: (i) introducing an effective amount of aptamer 9.3t to one or more sample(s) comprising an endogenous Factor IX protein of a mammalian subject (e.g., one or more sample(s) from the subject, e.g., a blood sample, a serum sample, etc.), and then assaying for the presence of clotting activity in the one or more sample(s) comprising said effective amount of aptamer 9.3t, wherein the presence of clotting activity in the one or more sample(s) comprising aptamer 9.3t indicates resistance of the endogenous Factor IX to inhibition by aptamer 9.3t and wherein the lack of clotting activity in the one or more sample(s) comprising aptamer 9.3t indicates sensitivity of the endogenous Factor IX to inhibition by aptamer 9.3t, as compared to a control sample (e.g., a sample not comprising aptamer 9.3t); and (ii) identifying the endogenous Factor IX comprised in the one or more sample(s) comprising an effective amount of aptamer 9.3t as sensitive to aptamer 9.3t (i.e., an aptamer-sensitive endogenous Factor IX), or identifying the endogenous Factor X comprised in the one or more sample(s) unable to determine an effective amount of aptamer 9.3t as resistant to aptamer 9.3t (i.e., an aptamer-resistant endogenous Factor IX).

In some embodiments, a method of the present invention may further comprise the step of determining the effective amount of aptamer 9.3t for reducing clotting activity of the endogenous Factor IX by (i) introducing a first amount of aptamer 9.3t to the one or more sample(s) comprising an endogenous Factor IX protein of a mammalian subject (e.g., one or more sample(s) from the subject, e.g., a blood sample, a serum sample, etc.), and assaying for the presence of clotting activity in the one or more sample(s) comprising said first amount of aptamer 9.3t, wherein the presence of clotting activity in the one or more sample(s) comprising aptamer 9.3t indicates resistance of the endogenous Factor IX to inhibition by aptamer 9.3t and wherein the lack of clotting activity in the one or more sample(s) comprising aptamer 9.3t indicates sensitivity of the endogenous Factor IX to inhibition by aptamer 9.3t, as compared to a control sample (e.g., a sample not comprising aptamer 9.3t); and then (ii) repeating step (i) with an escalating amount of aptamer 9.3t until: (a) an escalating amount of aptamer 9.3t reduces activity of the endogenous Factor IX in the one or more sample(s) (e.g., by at least 50, 60, 70, 80, 90, 91, 92, 93, 94, 95, 96, 97, 98, 99, or 100% of uninhibited endogenous Factor IX activity), thereby determining the effective amount of aptamer 9.3t for the one or more sample, or (b) no escalating amount of aptamer 9.3t reduces activity of the endogenous Factor IX in the one or more sample(s) (e.g., by at least 50, 60, 70, 80, 90, 91, 92, 93, 94, 95, 96, 97, 98, 99, or 100% of uninhibited endogenous target protein activity).

Another aspect of the present invention comprises a method of identifying a Factor IX protein as resistant to inhibition by aptamer 9.3t, comprising: (i) introducing an amount of aptamer 9.3t to one or more sample(s) comprising an endogenous Factor IX protein of a mammalian subject (e.g., one or more sample(s) from the subject, e.g., a blood sample, a serum sample, etc.), and then assaying for the presence of clotting activity in the one or more sample(s) comprising said amount of aptamer 9.3t as compared to a control sample (e.g., a sample not comprising aptamer 9.3t), wherein the presence of clotting activity in the one or more sample(s) comprising aptamer 9.3t indicates resistance of the endogenous Factor IX to inhibition by aptamer 9.3t at said amount and wherein the lack of clotting activity in the one or more sample(s) comprising aptamer 9.3t indicates sensitivity of the endogenous Factor IX to inhibition by aptamer 9.3t at said amount, as compared to a control sample; (ii) determining an effective amount of aptamer 9.3t for reducing clotting activity of the endogenous Factor IX by repeating step (i) with an escalating amount of aptamer 9.3t until: (a) an escalating amount of aptamer 9.3t reduces activity of the endogenous Factor IX in the one or more sample(s) by at least 50, 60, 70, 80, 90, 91, 92, 93, 94, 95, 96, 97, 98, 99, or 100% of uninhibited endogenous Factor IX activity, thereby determining the effective amount of aptamer 9.3t for the one or more sample, or (b) no escalating amount of aptamer 9.3t reduces activity of the endogenous Factor IX in the one or more sample(s) by at least 50, 60, 70, 80, 90, 91, 92, 93, 94, 95, 96, 97, 98, 99, or 100% of uninhibited endogenous Factor IX protein activity; and (iii) identifying the endogenous Factor IX comprised in the one or more sample(s) comprising an effective amount of aptamer 9.3t as sensitive to aptamer 9.3t (i.e., an aptamer-sensitive endogenous Factor IX), or identifying the endogenous Factor X comprised in the one or more sample(s) unable to determine an effective amount of aptamer 9.3t as resistant to aptamer 9.3t (i.e., an aptamer-resistant endogenous Factor IX).

In some embodiments, a method of the present invention may further comprise the steps of: (i) administering to a subject an effective amount of aptamer 9.3t, wherein the effective amount of aptamer 9.3t reduces activity of endogenous Factor IX protein; and (ii) administering to the subject an effective amount of the Factor IX protein identified as resistant to aptamer 9.3t.

Further provided herein is a recombinant Factor IX protein identified as resistant to aptamer 9.3t by the methods of the present invention.

Another aspect of the present invention provides an in vitro method of modeling protein and/or gene replacement therapy for treating a bleeding disorder in one or more non-hemophilic subject, comprising: (i) providing a sample from one or more protein non-deficient subject comprising an endogenous Factor IX protein; (ii) contacting the sample with an effective amount of aptamer 9.3t, wherein the effective amount of the aptamer reduces activity of the endogenous Factor IX protein as compared to uninhibited endogenous Factor IX protein activity (e.g., reduces activity by at least 50, 60, 70, 80, 90, 91, 92, 93, 94, 95, 96, 97, 98, 99, or 100% of uninhibited endogenous Factor IX protein activity); and (iii) contacting the sample with an effective amount of an exogenous aptamer-resistant replacement Factor IX protein, nucleic acid molecule (e.g., mRNA), vector, cell, and/or composition (e.g., pharmaceutical composition) of the present invention.

Also provided in the present invention is an in vitro method of identifying genetic background effects of one or more non-hemophilic subject on an exogenous Factor IX protein, comprising the steps of: (i) providing a sample from one or more non-hemophilic subject comprising an endogenous Factor IX protein; (ii) contacting the sample with an effective amount of aptamer 9.3t, wherein the effective amount of the aptamer 9.3t reduces activity of the endogenous Factor IX protein as compared to uninhibited endogenous Factor IX protein activity (e.g., reduces activity by at least 50, 60, 70, 80, 90, 91, 92, 93, 94, 95, 96, 97, 98, 99, or 100% of uninhibited endogenous Factor IX protein activity); (iii) contacting the sample with an effective amount of an exogenous aptamer-resistant replacement Factor IX protein, nucleic acid molecule, vector, cell, and/or composition of the present invention (e.g., a pharmaceutical composition); (iv) assaying for the presence of protein clotting activity in the sample comprising the exogenous aptamer-resistant replacement Factor IX protein and in a control (a sample from a subject of a different genetic background and contacted with the exogenous replacement Factor IX protein (e.g., a control/reference genetic background)); (v) comparing the relative level and/or presence of protein clotting activity of the sample comprising the exogenous aptamer-resistant replacement Factor IX protein and the control to identify genetic background effects of the subject on the efficacy and/or safety of the exogenous replacement Factor IX protein; and (vi) associating the difference in relative level and/or presence of the protein clotting activity with genetic background differences between the one or more subject and the control.

An additional aspect of the present invention provides a method of modeling a Factor IX protein and/or gene replacement therapy for treating a bleeding disorder in one or more non-hemophilic subject, comprising: (i) administering to the one or more subject an effective amount of aptamer 9.3t, wherein the effective amount of aptamer 9.3t reduces activity of endogenous Factor IX protein as compared to uninhibited endogenous Factor IX protein activity (e.g., reduces activity by at least 50, 60, 70, 80, 90, 91, 92, 93, 94, 95, 96, 97, 98, 99, or 100% of uninhibited endogenous Factor IX activity); (ii) administering to the one or more subject an effective amount of an exogenous aptamer-resistant replacement Factor IX protein, nucleic acid molecule, vector, cell, and/or composition of the present invention (e.g., a pharmaceutical composition).

In some embodiments, the methods of the present invention may further comprise the steps of: assaying for the presence of clotting activity in the subject comprising the exogenous Factor IX protein and in a control (e.g., a subject not administered an exogenous Factor IX); and comparing the relative presence of clotting activity of the subject comprising the exogenous Factor IX protein and the control to identify genetic background effects of the subject on the efficacy of the exogenous Factor IX protein.

In some embodiments, the bleeding disorder may be Factor IX deficiency (Hemophilia B).

Another aspect of the present invention provides a method of identifying genetic background effects on an exogenous Factor IX protein in one or more non-hemophilic subject, comprising the steps of: (i) administering to the one or more subject an effective amount of aptamer 9.3t, wherein the effective amount of aptamer 9.3t reduces activity of endogenous Factor IX protein as compared to uninhibited endogenous Factor IX protein activity (e.g., reduces activity by at least 50, 60, 70, 80, 90, 91, 92, 93, 94, 95, 96, 97, 98, 99, or 100% of uninhibited endogenous Factor IX activity); (ii) administering to the one or more subject an effective amount of an exogenous aptamer-resistant replacement Factor IX protein, nucleic acid molecule, vector, cell, and/or composition of the present invention (e.g., a pharmaceutical composition); (iii) assaying for the presence of clotting activity in the subject comprising the exogenous Factor IX protein and in a control (e.g., a subject not administered an exogenous Factor IX); (iv) comparing the relative presence of clotting activity of the one or more subject comprising the exogenous Factor IX protein and the control to identify genetic background effects of the subject on the efficacy of the exogenous Factor IX protein; and (v) associating the difference in relative level and/or presence of the clotting activity with genetic background differences between the one or more subject and the control.

In some embodiments, the one or more non-hemophilic subject may be two or more subjects of different genetic backgrounds (e.g. Collaborative cross mouse strains).

In some embodiments, the associating step may comprise associating the difference in relative level and/or presence of the protein clotting activity with known genetic background differences between the one or more subject and the control.

In some embodiments, the associating step may comprise a step of determining genetic background differences between the one or more subject and the control, and then associating the difference in relative level and/or presence of the protein clotting activity with determined genetic background differences between the subject and the control.

In some embodiments, the administering step may comprise administering to the one or more subject an effective amount of an exogenous Factor IX protein comprises administering a plurality of recombinant and/or exogenous replacement Factor IX proteins (e.g., replacement Factor IX proteins comprising modifications (mutations, substitutions, deletions, insertions, post-translational modifications, etc., such as for example FIX Padua mutation, codon-optimization, 2xFc fusion FIX) to be screened). In some embodiments, administering an effective amount of an exogenous Factor IX protein may comprise administering an effective amount of a nucleic acid molecule (e.g., an mRNA), vector, cell, and/or composition of the invention encoding and/or comprising the exogenous Factor IX protein.

In some embodiments, the methods of the present invention may further comprise determining a safety profile of the administered exogenous Factor IX replacement protein (e.g., the plurality of recombinant and/or exogenous replacement proteins, e.g., replacement proteins comprising modifications (mutations, substitutions, deletions, insertions, post-translational modifications, etc.) to be screened).

In some embodiments, determining a safety profile may comprise screening for biological responses to the administered recombinant Factor IX protein, such as but not limited to an antibody response raised against the recombinant Factor IX protein, a cellular immune response raised against the recombinant Factor IX protein, and/or development of thrombo-emboli in response to the recombinant Factor IX protein.

In some embodiments, the methods of the present invention may further comprise determining efficacy of the administered exogenous Factor IX replacement protein (e.g., the plurality of recombinant and/or exogenous Factor IX proteins, e.g., recombinant Factor IX proteins comprising modifications (mutations, substitutions, deletions, insertions, post-translational modifications, etc.) to be screened).

In some embodiments, determining efficacy may comprise screening for biological effects of the administered exogenous Factor IX replacement protein, such as but not limited to concentration over time of the recombinant Factor IX protein, functional activity of the recombinant Factor IX protein, etc.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1: A General Aptamer-Based Methodology to Evaluate Protein and Gene Replacement Therapies in Animal Models Premised on Wild-Type Animals

The present invention utilizes specificity of aptamer binding to facilitate efficient inhibition of natural proteins in vivo and in vitro in a species-specific manner. These studies showed that species-specific aptamer-binding to target molecules can be employed to distinguish between endogenous aptamer-sensitive host proteins and exogenously delivered aptamer-resistant proteins. Consequently, once identified, aptamer-resistant non-human proteins can be employed to model gene and protein replacement therapies in a variety of host species whose cognate proteins are aptamer-sensitive.

This approach establishes transient functional KO models of WT proteins and thereby facilitates functional characterization of either natural or modified proteins and their delivery in healthy animals.

The disclosed method comprises two steps including:
A) zoo screening of orthologous aptamer-sensitive and aptamer-resistant proteins. Species comprising either aptamer-sensitive or aptamer-resistant proteins are identified as putative host (aptamer-sensitive) or as the source of the relevant exogenous genes/proteins (aptamer-resistant), respectively; and
B) optimization of the identified aptamer-resistant proteins to function in the relevant (aptamer-sensitive) host environment.

### Example 2: A Proof of Principle Model of Factor IX Aptamer-Resistant Protein Replacement in a Non-Hemophilic Subject

As a proof of principle of the above novel methodology, this study established an aptamer-based gene replacement therapy of Factor IX in a hemophilia B (HB) model in three strains of non-hemophilic mice. These novel gene replacement models facilitated characterization of sex and host genetic background effects on the efficacy and safety of Factor IX gene delivery.

Additionally described are novel factor IX (FIX) cDNAs, which support in vivo modeling of gene and protein replacement therapeutic protocols for hemophilia B in non-hemophilic animals. One novel cDNA (cTK1908) encodes a unique modified rabbit FIX protein which exhibits high specific clotting activity, resistance to the inhibitory activity of the 9.3t RNA aptamer, and could be serologically quantified in the presence of host FIX proteins.

The 9.3t aptamer is a modified 35-nucleotide truncate of aptamer 9.3 containing 5′ cholesterol-tetraethylene glycol (5CholTEG), 3′ inverted deoxythymidine (3InvdT, n=1), regular purines (rA or rG, n=15), and internal 2′ fluoropyrimidines (12FU or i2FC, n=19); the 2′ fluoro bases have a fluorine modified ribose which increases affinity binding and improves stability of aptamers in plasma/serum environment. Aptamer 9.3tM cannot efficiently bind FIX proteins and serves as a negative control. 9.3tM aptamer has two point mutations that differ from its active counterpart 9.3t. Both aptamers were purchased from Integrated DNA Technologies (IDT™). These RNA aptamers and their sequences are further described as reversible antagonists of coagulant IXa in Rusconi et al (2002) Nature 419:90-94; incorporated herein by reference.

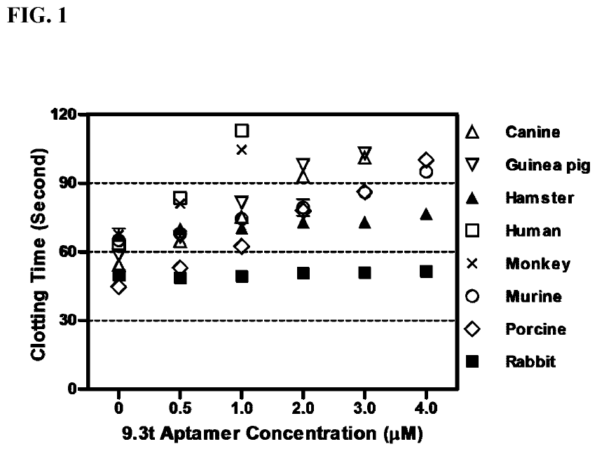

To identify naturally aptamer-resistant FIX proteins, the clotting activity of FIX was evaluated in plasma obtained from eight different species (Canine, Guinea pig, Hamster, Human, Monkey, Murine, Porcine and Rabbit) either in the absence or presence of increasing concentrations of the 9.3t aptamer. Activated partial thromboplastin time assays (aPTT) were employed to test the hypothesis that the efficacy of the 9.3t aptamer at inhibiting factor IX clotting activity is species-dependent. To this end, aPTT assays were conducted with normal plasma obtained from each of the eight species in the presence of increasing concentrations of the 9.3t aptamer. As shown in FIG. 1, increasing concentrations of the 9.3t aptamers had no or minimal effect on rabbit and hamster aPTT, respectively. However, clotting activity in Canine, Human, Simian, Guineapig, and murine plasma samples was efficiently inhibited by increasing concentration of the 9.3t aptamer (0.5-4.0 μM). Accordingly, the results indicated that both the rabbit and the hamster FIX proteins were resistant to the inhibitory effects of the 9.3t aptamer (FIG. 1) and thus can be employed to differentiate between clotting activity mediated by host/endogenous FIX proteins which are aptamer sensitive, and exogenously delivered aptamer-resistant FIX cDNAs/proteins.

It was hypothesized that in order to facilitate in vivo modeling of gene and protein replacement therapies, exogenous aptamer-resistant FIX proteins should exhibit high specific activity and be serologically distinguishable from the relevant host wildtype FIXs. Further, optimization of the rabbit FIX activation process by Factor XIa could enhance the specific activity of FIX in relevant experimental systems which would use non-rabbit plasma (e.g., mouse, human simian plasma). Replacement of rabbit FIX sequences encoding either the XIa cleavage site-α and its neighboring amino acids, or the XIa cleavage site-β and its neighboring amino acids or both (sites a and B and their neighboring amino acids) enhanced the specific activity of the resultant recombinant rabbit FIX.

Among the constructs generated was a modified rabbit FIX (SEQ ID NO:13) encoded by cDNA cTK1904 (SEQ ID NO:20) in which a DNA fragment encoding the amino acid target-sequence of the human XIa and its neighboring amino acids in the human FIX protein (SVSQTSKLTRAETVFPDVD; SEQ ID NO:7) replaced the cognate sequence in the rabbit FIX protein (SVSHASKKITRATTIFSNTE; SEQ ID NO:3). The human cleavage site a sequence is one amino acid residue less than that of the cognate sequence in the rabbit. To maximize transgene expression, the modified rabbit FIX cDNA was codon optimized and comprised a mutation similar to the human FIX variant R338L (known as the Padua FIX; see, e.g., Simioni et al. 2009 N. Engl. J. Med. 361 (17): 1671-1675, the disclosures of which are incorporated herein by reference in their entirety).

To facilitate quantification of the chimeric rabbit/human FIX protein concentration in the presence of various normal host FIX proteins, a DNA fragment encoding 4 x Myc-tag was cloned in frame to the 3′ end of the newly developed FIX open reading frame (ORF). To avoid potential inhibitory effects of the Myc-tags on FIX clotting activity, a second DNA fragment encoding the XIa amino acid target sequence (SVSQTSKLTRAETVFPDVD; SEQ ID NO: 7) was cloned between the 3′ end of the novel rabbit FIX open-reading frame and the 5′ end of the 4 x Myc-tag encoding sequence.

The sequences of the resultant recombinant proteins from each of these modifications is described below.

Modified Rabbit Padua FIX comprising the human
activation peptide and cleavage sites α and β,
additional site α and myc tag at end, and Padua
substitution(position 384).
SEQ ID NO: 12
MQFLNTIMAESPGLITICLLGYLLSAECTVFLDHENATKILNRAK
RYNSGKLEEFVSGNLERECIEERCSFEEAREVFENTEKTTEFWKQ
YVDGDQCESNPCLNGGSCKDDINAYECWCQYGFEGKNCELDSTCS
IKNGRCEQFCRKNRNNKIICSCTEGYRLAENQKSCEPAVPFPCGR
VSVSQTSKLTRAETVFPDVDYENFTEAETILDNITQSTQSFNDFT
RVVGGEDAKPGQFPWQVLLNGKVEAFCGGSIINEKWVVTAAHCIK
PDDNITVVAGEYNIQETENTEQKRNVIRIIPYHKYNATINKYNHD
IALLELDKPLTLNSYVTPICIANREYTNIFLNFGSGYVSGWGRVF
NRGRQASILQYLRVPFVDRATCLLSTKFTIYNNMFCAGFDVGGKD
SCEGDSGGPHVTEVEGTSFLTGIISWGEECAIKGKYGVYTRVSWY
VNWIKEKTKLTSVSQTSKLTRAETVFPDVDGSEQKLISEEDLEQK
LISEEDLEQKLISEEDLEQKLISEEDL.
Modified Rabbit Padua FIX comprising human
cleavage site α2 additional human cleavage
site α and myc tag at end, and Padua
substitution (position 384); sequence
encoded by cDNA c1904.
SEQ ID NO: 13
MQFLNTIMAESPGLITICLLGYLLSAECTVFLDHENATKILNRAK
RYNSGKLEEFVSGNLERECIEERCSFEEAREVFENTEKTTEFWKQ
YVDGDQCESNPCLNGGSCKDDINAYECWCQYGFEGKNCELDSTCS
IKNGRCEQFCRKNRNNKIICSCTEGYRLAENQKSCEPAVPFPCGR
VSVSQTSKLTRAETVFPDVDYENFTEAETIRGNVTQDAQSSDDFT
RIVGGENAKPGQFPWQVLLNGKVEAFCGGSIINEKWVVTAAHCIK
PDDNITVVAGEYNIQETENTEQKRNVIRIIPYHKYNATINKYNHD
IALLELDKPLTLNSYVTPICIANREYTNIFLNFGSGYVSGWGRVF
NRGRQASILQYLRVPFVDRATCLLSTKFTIYNNMFCAGFDVGGKD
SCEGDSGGPHVTEVEGTSFLTGIISWGEECAIKGKYGVYTRVSWY
VNWIKEKTKLTSVSQTSKLTRAETVFPDVDGSEQKLISEEDLEQK
LISEEDLEQKLISEEDLEQKLISEEDL.
Modified Rabbit Padua FIX comprising human
cleavage site β and additional human cleavage
site α and myc tag at end, and Padua
substitution (position 385).
SEQ ID NO: 14
MQFLNTIMAESPGLITICLLGYLLSAECTVFLDHENATKILNRAK
RYNSGKLEEFVSGNLERECIEERCSFEEAREVFENTEKTTEFWKQ
YVDGDQCESNPCLNGGSCKDDINAYECWCQYGFEGKNCELDSTCS
IKNGRCEQFCRKNRNNKIICSCTEGYRLAENQKSCEPAVPFPCGR
VSVSHASKKITRATTIFSNTEYENFTEAETILDNITQSTQSFNDF
TRVVGGEDAKPGQFPWQVLLNGKVEAFCGGSIINEKWVVTAAHCI
KPDDNITVVAGEYNIQETENTEQKRNVIRIIPYHKYNATINKYNH
DIALLELDKPLTLNSYVTPICIANREYTNIFLNFGSGYVSGWGRV
FNRGRQASILQYLRVPFVDRATCLLSTKFTIYNNMFCAGFDVGGK
DSCEGDSGGPHVTEVEGTSFLTGIISWGEECAIKGKYGVYTRVSW
YVNWIKEKTKLTSVSQTSKLTRAETVFPDVDGSEQKLISEEDLEQ
KLISEEDLEQKLISEEDLEQKLISEEDL.

Additional modified and/or chimeric Rabbit Factor IX proteins generated in the present invention include the below.

Modified Rabbit Padua FIX comprising additional
human cleavage site α and myc tag at end.
(Padua at 385) encoded by cDNA c1847)
SEQ ID NO: 15
MQFLNTIMAESPGLITICLLGYLLSAECTVFLDHENATKILNRAK
RYNSGKLEEFVSGNLERECIEERCSFEEAREVFENTEKTTEFWKQ
YVDGDQCESNPCLNGGSCKDDINAYECWCQYGFEGKNCELDSTCS
IKNGRCEQFCRKNRNNKIICSCTEGYRLAENQKSCEPAVPFPCGR
VSVSHASKKITRATTIFSNTEYENFTEAETIRGNVTQDAQSSDDF
TRIVGGENAKPGQFPWQVLLNGKVEAFCGGSIINEKWVVTAAHCI
KPDDNITVVAGEYNIQETENTEQKRNVIRIIPYHKYNATINKYNH
DIALLELDKPLTLNSYVTPICIANREYTNIFLNFGSGYVSGWGRV
FNRGRQASILQYLRVPFVDRATCLLSTKFTIYNNMFCAGFDVGGK
DSCEGDSGGPHVTEVEGTSFLTGIISWGEECAIKGKYGVYTRVSW
YVNWIKEKTKLTSVSQTSKLTRAETVFPDVDGSEQKLISEEDLEQ
KLISEEDLEQKLISEEDLEQKLISEEDL.
Chimeric Human/Rabbit Padua FIX comprising
179 N-terminal human amino acids and Padua at
385, encoded by cDNA c1835
SEQ ID NO: 16
MQRVNMIMAESPGLITICLLGYLLSAECTVFLDHENANKILNRPK
RYNSGKLEEFVQGNLERECMEEKCSFEEAREVFENTERTTEFWKQ
YVDGDQCESNPCLNGGSCKDDINSYECWCPFGFEGKNCELDVTCN
IKNGRCEQFCKNSADNKVVCSCTEGYRLAENQKSCEPAVPFPCGG
VSVSHASKKITRATTIFSNTEYENFTEAETIRGNVTQRSQSSDDF
TRIVGGENAKPGQFPWQVLLNGKVEAFCGGSIINEKWVVTAAHCI
KPDDNITVVAGEYNIQETENTEQKRNVIRIIPYHKYNATINKYNH
DIALLELDKPLTLNSYVTPICIANREYTNIFLNFGSGYVSGWGRV
FNRGRQASILQYLRVPFVDRATCLLSTKFTIYNNMFCAGFDVGGK
DSCEGDSGGPHVTEVEGTSFLTGIISWGEECAIKGKYGVYTRVSW
YVNWIKEKTKLT.

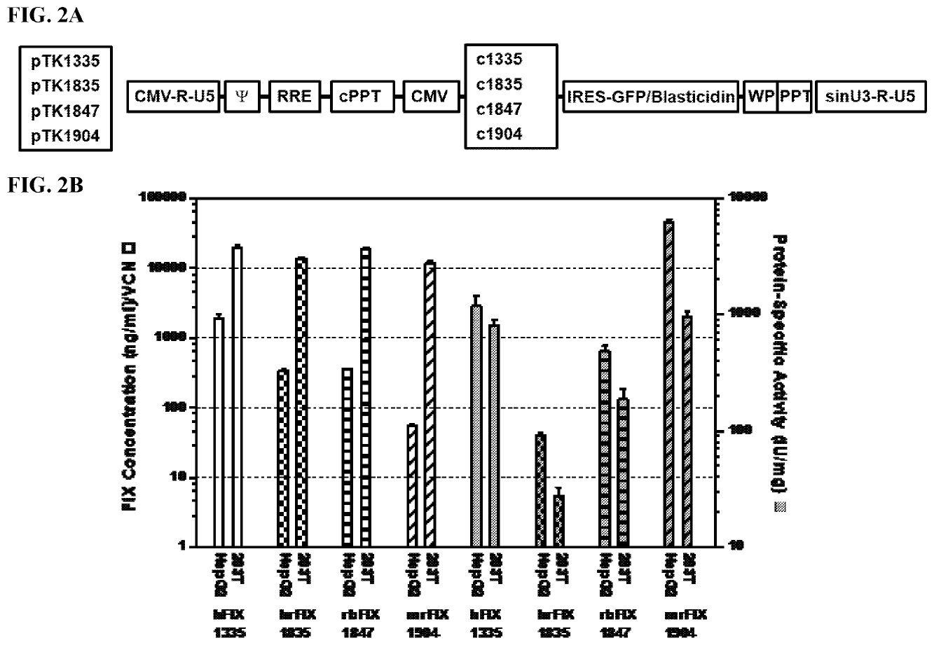

FIG. 2A is a schematic of the structure of different lentiviral vectors employed to deliver the wild type and modified FIX proteins to HepG2 and 293T cells for measurement of vector and protein specific activities. As shown in FIG. 2B, the modified rabbit FIX (cTK1904) protein was efficiently detected by ELISA assays and its clotting specific activity of was comparable and significantly superior to that of the codon optimized human (cTK1335) and rabbit (cTK1847) FIX proteins carrying the Padua mutation, respectively.

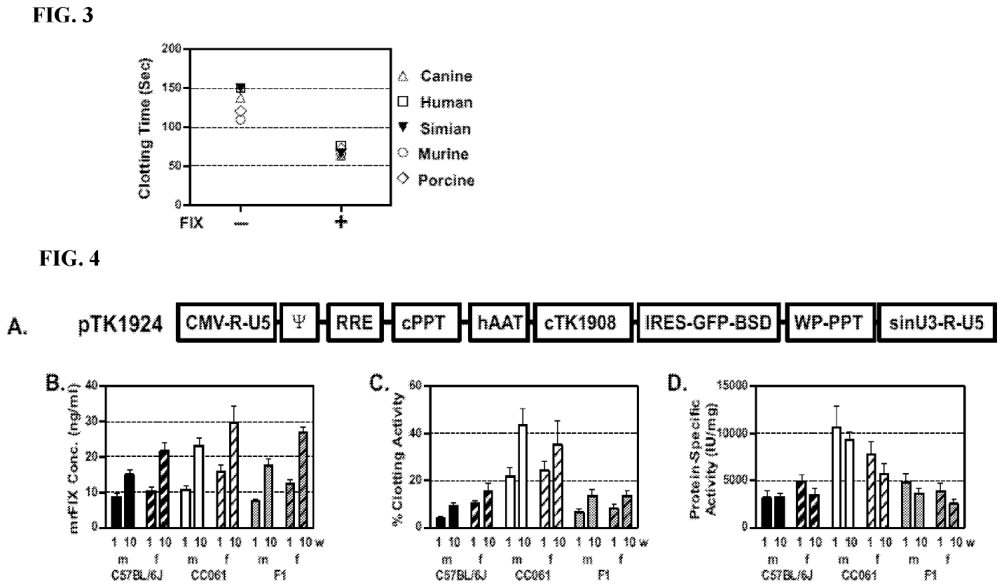

Importantly, the modifications incorporated to the newly developed rFIX (cTK1904) did not affect its ability to mediate efficient clotting activity in the presence of the 9.3t aptamer. As shown in FIG. 3, the modified rabbit rFIX (cTK1904) abolished the inhibitory effects of the 9.3t aptamer in various aptamer-sensitive plasma samples obtained from different animals.

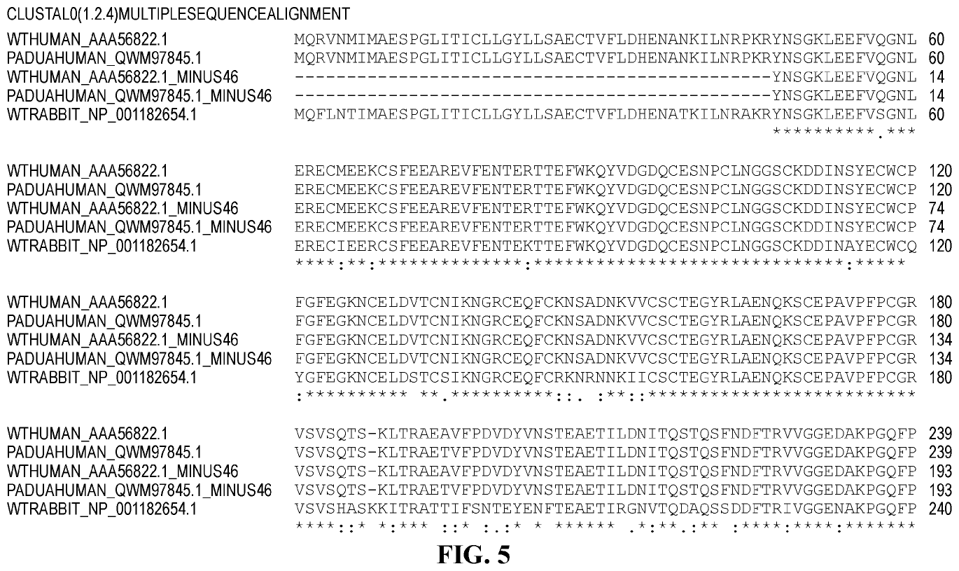

The ability to efficiently quantify the clotting activity of the novel aptamer-resistant FIX protein following gene or protein replacement therapy enhances its usefulness in preclinical studies. Quantification of the aptamer-resistant FIX clotting activity could be determined by aPTT assay, using plasma samples from animals subjected to gene or protein replacement protocols as described herein in the presence of the 9.3t aptamer. In one approach, the aptamer may be delivered directly to the treated animals as described earlier by Rusconi et al. (2004, Nat Biotechnol 22 (11): 1423-1428; the disclosures of which are incorporated herein by reference in their entirety). This approach is efficient; however, it requires relatively high doses of aptamers. Alternatively, as shown in FIG. 4 panels A-D, direct addition of the 9.3t aptamer to plasma samples obtained from animals subjected to either gene or protein replacement protocols achieves inhibitory aptamer concentrations of (4 to 8 μM) with minimal aptamer doses.

### Example 3: A Novel Factor IX Whose Half Li is Extended by Two Fc Domains

While not wishing to be bound to theory, the lack of a non-human primate model to hemophilia B may limit the ability to accurately characterize the safety and efficacy of novel factor IX proteins prior to clinical trials. Proposed herein is a general approach to model gene and protein replacement therapies in healthy (wild type) animals which may facilitate preclinical research of multiple rare diseases to which genetically based animal models do not exist. A proof-of-principle approach to model gene and protein therapies in non-hemophilic animals is based on an aptamer to human factor IX. The specificity of aptamer binding may facilitate efficient inhibition of host proteins in vivo and in vitro in a species-specific manner. Therefore, species-specific aptamer-binding to target molecules may be employed to distinguish between endogenous aptamer-sensitive host proteins and exogenously delivered aptamer-resistant proteins. Consequently, once identified, aptamer-resistant non-human proteins may be employed to model gene and protein replacement therapies in a variety of host species whose cognate proteins are aptamer-sensitive. Practically, the disclosed approach establishes transient functional KO of WT proteins and thereby facilitates functional characterization of either natural or modified proteins and their delivery in healthy animals.

In this study, it was sought to extend factor IX half-life in vivo. A tandem of two fragment crystallizable (Fc) regions separated by a poly-glycine linker were fused to human factor IX. Each Fc region comprised a hinge domain (H) and two heavy chain constant domains (CH2 and CH3) (FIG. 6; nucleotide sequence provided in SEQUENCES section as SEQ ID NO:26).

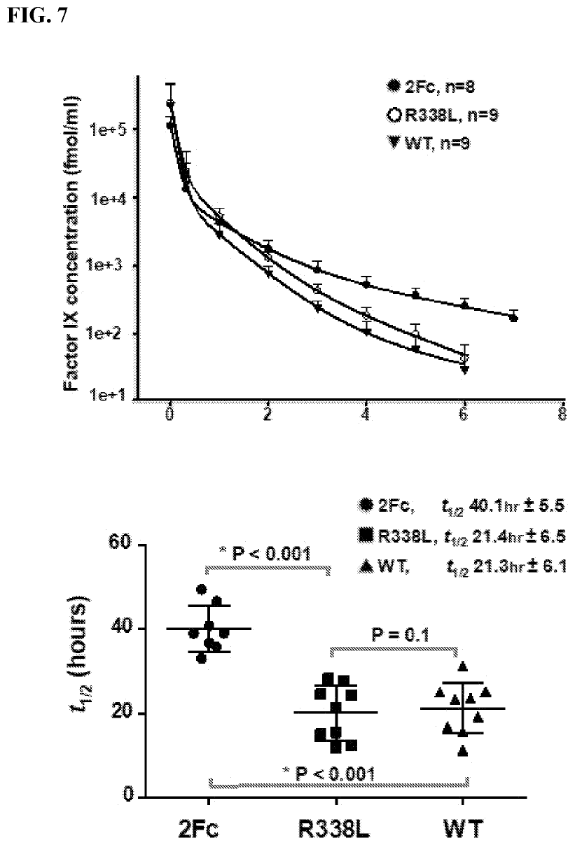

Due to the binding of the fused Fc domains to neonatal Fc receptors (FcRn), the complexes of FcRn-factor IX fusion are sorted to recycling endosomes intracellularly (e.g., in vivo and/or in vitro) and released to the circulation leading to dissociation of the FcRn-Fc/factor IX complex, thereby extending the half-life of the protein (Shapiro, A. Expert Opin Biol Ther, 2013. 13 (9): 1287-97). In verification thereof, following injection to hemophilia B mice, the novel human factor IX/2Fc domains fusion protein exhibited significantly extended half-life (FIG. 7).

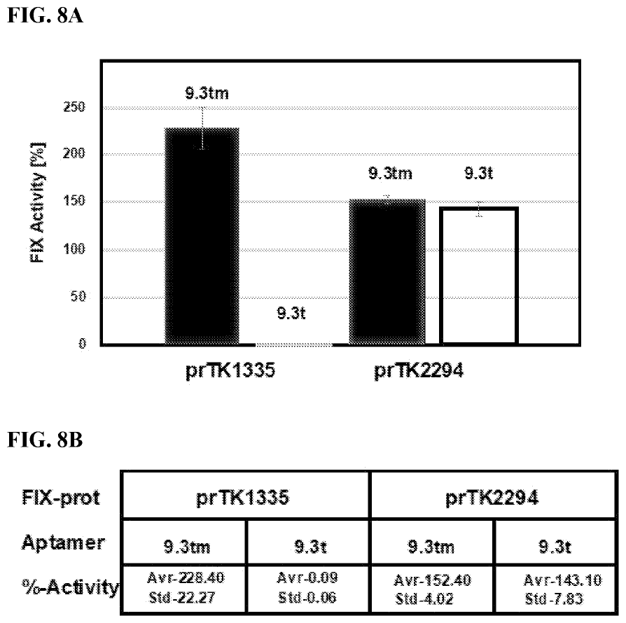

This half-life extended fusion protein (prTK2294) comprises an aptamer resistant rabbit-based factor IX (FIGS. 8A-8B), and a tandem of two fragment crystallizable (Fc) domains. The 2294 protein is made of 2 parts: The aptamer resistant factor IX (N-terminus (the start of the protein)) and the tandem of two Fc domains (C-terminus (the end of the protein)). Between these two parts, the target sequence to the human XIa was included followed by a poly glycine linker. This new fusion protein combines two technologies which together facilitate characterization of factor IX proteins with extended half-life in non-hemophilic animals.

The foregoing is illustrative of the present invention, and is not to be construed as limiting thereof. The invention is defined by the following claims, with equivalents of the claims to be included therein.

TABLE 1

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 2

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | aIle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

### SEQUENCES

hFIX (GenBank® Accession No. AAA56822.1, lacking
first 46 residuescorresponding to signal
peptide and protein peptide; reference sequence
for Padua R338L numbering)
SEQ ID NO: 1
YNSGKLEEFVQGNLERECMEEKCSFEEAREVFENTERTTEFWKQYVDGDQCESNPCLNGGSCKDDINSYE
CWCPFGFEGKNCELDVTCNIKNGRCEQFCKNSADNKVVCSCTEGYRLAENQKSCEPAVPFPCGRVSVSQT
SKLTRAEAVFPDVDYVNSTEAETILDNITQSTQSFNDFTRVVGGEDAKPGQFPWQVVLNGKVDAFCGGSI
VNEKWIVTAAHCVETGVKITVVAGEHNIEETEHTEQKRNVIRIIPHHNYNAAINKYNHDIALLELDEPLV
LNSYVTPICIADKEYTNIFLKFGSGYVSGWGRVFHKGRSALVLQYLRVPLVDRATCLRSTKFTIYNNMFC
AGFHEGGRDSCQGDSGGPHVTEVEGTSFLTGIISWGEECAMKGKYGIYTKVSRYVNWIKEKTKLT.
WT rabbit FIX (does not contain Padua; wt “R” in
place of Paduahighlighted; position 385 in TK seqs);
GenBank® Accession No. NP_001182654.1
SEQ ID NO: 2
MQFLNTIMAESPGLITICLLGYLLSAECTVFLDHENATKILNRAKRYNSGKLEEFVSGNLERECIEERCS
FEEAREVFENTEKTTEFWKQYVDGDQCESNPCLNGGSCKDDINAYECWCQYGFEGKNCELDSTCSIKNGR
CEQFCRKNRNNKIICSCTEGYRLAENQKSCEPAVPFPCGRVSVSHASKKITRATTIFSNTEYENFTEAET
IRGNVTQDAQSSDDFTRIVGGENAKPGQFPWQVLLNGKVEAFCGGSIINEKWVVTAAHCIKPDDNITVVA
GEYNIQETENTEQKRNVIRIIPYHKYNATINKYNHDIALLELDKPLTLNSYVTPICIANREYTNIFLNFG
SGYVSGWGRVFNRGRQASILQYLRVPFVDRATCLRSTKFTIYNNMFCAGFDVGGKDSCEGDSGGPHVTEV
EGTSFLTGIISWGEECAIKGKYGVYTRVSWYVNWIKEKTKLT.
Rabbit cleavage site αlpha (position 182-201 in SEQ ID NO: 2)
SEQ ID NO: 3
SVSHASKKITRATTIFSNTE.
Rabbit cleavage site beta (position 212-233 in SEQ ID NO: 2)
SEQ ID NO: 4
RGNVTQDAQSSDDFTRIVGGEN.
Rabbit active peptide (position 193-227 in SEQ ID NO: 2)
SEQ ID NO: 5
ATTIFSNTEYENFTEAETIRGNVTQDAQSSDDFTR.
Rabbit alpha cleavage, active protein, and beta cleavage
SEQ ID NO: 6
SVSHASKKITRATTIFSNTEYENFTEAETIRGNVTQDAQSSDDFTRIVGGEN.
Human alpha cleavage site
SEQ ID NO: 7
SVSQTSKLTRAETVFPDVD.
Human beta cleavage site
SEQ ID NO: 8
LDNITQSTQSFNDFTRVVGGED.
Human active protein
SEQ ID NO: 9
AETVFPDVDYVNSTEAETILDNITQSTQSFNDFTR.
Human alpha cleavage, active protein, and beta cleavage
SEQ ID NO: 10
SVSQTSKLTRAETVFPDVDYVNSTEAETILDNITQSTQSFNDFTRVVGGED
Myc tag
SEQ ID NO: 11
EQKLISEEDLEQKLISEEDLEQKLISEEDLEQKLISEEDL.
Modified Rabbit Padua FIX comprising human cleavage
site α, humanactivation peptide, and human cleavage
site β and additional human cleavage site α andmyc
tag at end. (Padua at 384; with human insertions)
SEQ ID NO: 12
MQFLNTIMAESPGLITICLLGYLLSAECTVFLDHENATKILNRAKRYNSGKLEEFVSGNLERECIEERCS
FEEAREVFENTEKTTEFWKQYVDGDQCESNPCLNGGSCKDDINAYECWCQYGFEGKNCELDSTCSIKNGR
CEQFCRKNRNNKIICSCTEGYRLAENQKSCEPAVPFPCGRVSVSQTSKLTRAETVFPDVDYENFTEAETI
LDNITQSTQSFNDFTRVVGGEDAKPGQFPWQVLLNGKVEAFCGGSIINEKWVVTAAHCIKPDDNITVVAG
EYNIQETENTEQKRNVIRIIPYHKYNATINKYNHDIALLELDKPLTLNSYVTPICIANREYTNIFLNFGS
GYVSGWGRVFNRGRQASILQYLRVPFVDRATCLLSTKFTIYNNMFCAGFDVGGKDSCEGDSGGPHVTEVE
GTSFLTGIISWGEECAIKGKYGVYTRVSWYVNWIKEKTKLTSVSQTSKLTRAETVFPDVDGSEQKLISEE
DLEQKLISEEDLEQKLISEEDLEQKLISEEDL.
Modified Rabbit Padua FIX comprising human cleavage
site α andadditional human cleavage site α and myc
tag at end. (Padua at 384; with human insertions)
encoded by cDNA c1904
SEQ ID NO: 13
MQFLNTIMAESPGLITICLLGYLLSAECTVFLDHENATKILNRAKRYNSGKLEEFVSGNLERECIEERCS
FEEAREVFENTEKTTEFWKQYVDGDQCESNPCLNGGSCKDDINAYECWCQYGFEGKNCELDSTCSIKNGR
CEQFCRKNRNNKIICSCTEGYRLAENQKSCEPAVPFPCGRVSVSQTSKLTRAETVFPDVDYENFTEAETI
RGNVTQDAQSSDDFTRIVGGENAKPGQFPWQVLLNGKVEAFCGGSIINEKWVVTAAHCIKPDDNITVVAG
EYNIQETENTEQKRNVIRIIPYHKYNATINKYNHDIALLELDKPLTLNSYVTPICIANREYTNIFLNFGS
GYVSGWGRVFNRGRQASILQYLRVPFVDRATCLLSTKFTIYNNMFCAGFDVGGKDSCEGDSGGPHVTEVE
GTSFLTGIISWGEECAIKGKYGVYTRVSWYVNWIKEKTKLTSVSQTSKLTRAETVFPDVDGSEQKLISEE
DLEQKLISEEDLEQKLISEEDLEQKLISEEDL.
Modified Rabbit Padua FIX comprising human cleavage
site β andadditional human cleavage site α and myc
tag at end. (Padua at 385)
SEQ ID NO: 14
MQFLNTIMAESPGLITICLLGYLLSAECTVFLDHENATKILNRAKRYNSGKLEEFVSGNLERECIEERCS
FEEAREVFENTEKTTEFWKQYVDGDQCESNPCLNGGSCKDDINAYECWCQYGFEGKNCELDSTCSIKNGR
CEQFCRKNRNNKIICSCTEGYRLAENQKSCEPAVPFPCGRVSVSHASKKITRATTIFSNTEYENFTEAET
ILDNITQSTQSFNDFTRVVGGEDAKPGQFPWQVLLNGKVEAFCGGSIINEKWVVTAAHCIKPDDNITVVA
GEYNIQETENTEQKRNVIRIIPYHKYNATINKYNHDIALLELDKPLTLNSYVTPICIANREYTNIFLNFG
SGYVSGWGRVFNRGRQASILQYLRVPFVDRATCLLSTKFTIYNNMFCAGFDVGGKDSCEGDSGGPHVTEV
EGTSFLTGIISWGEECAIKGKYGVYTRVSWYVNWIKEKTKLTSVSQTSKLTRAETVFPDVDGSEQKLISE
EDLEQKLISEEDLEQKLISEEDLEQKLISEEDL.
Modified Rabbit Padua FIX comprising additional
human cleavage site α and myc tag at end.
(Padua at 385) encoded by cDNA c1847)
SEQ ID NO: 15
MQFLNTIMAESPGLITICLLGYLLSAECTVFLDHENATKILNRAKRYNSGKLEEFVSGNLERECIEERCS
FEEAREVFENTEKTTEFWKQYVDGDQCESNPCLNGGSCKDDINAYECWCQYGFEGKNCELDSTCSIKNGR
CEQFCRKNRNNKIICSCTEGYRLAENQKSCEPAVPFPCGRVSVSHASKKITRATTIFSNTEYENFTEAET
IRGNVTQDAQSSDDFTRIVGGENAKPGQFPWQVLLNGKVEAFCGGSIINEKWVVTAAHCIKPDDNITVVA
GEYNIQETENTEQKRNVIRIIPYHKYNATINKYNHDIALLELDKPLTLNSYVTPICIANREYTNIFLNFG
SGYVSGWGRVFNRGRQASILQYLRVPFVDRATCLLSTKFTIYNNMFCAGFDVGGKDSCEGDSGGPHVTEV
EGTSFLTGIISWGEECAIKGKYGVYTRVSWYVNWIKEKTKLTSVSQTSKLTRAETVFPDVDGSEQKLISE
EDLEQKLISEEDLEQKLISEEDLEQKLISEEDL.
Chimeric Human/Rabbit Padua FIX comprising 179
N-terminal human amino acids and Padua at 385,
encoded by cDNA c1835
SEQ ID NO: 16
MQRVNMIMAESPGLITICLLGYLLSAECTVFLDHENANKILNRPKRYNSGKLEEFVQGNLERECMEEKCS
FEEAREVFENTERTTEFWKQYVDGDQCESNPCLNGGSCKDDINSYECWCPFGFEGKNCELDVTCNIKNGR
CEQFCKNSADNKVVCSCTEGYRLAENQKSCEPAVPFPCGGVSVSHASKKITRATTIFSNTEYENFTEAET
IRGNVTQRSQSSDDFTRIVGGENAKPGQFPWQVLLNGKVEAFCGGSIINEKWVVTAAHCIKPDDNITVVA
GEYNIQETENTEQKRNVIRIIPYHKYNATINKYNHDIALLELDKPLTLNSYVTPICIANREYTNIFLNFG
SGYVSGWGRVFNRGRQASILQYLRVPFVDRATCLLSTKFTIYNNMFCAGFDVGGKDSCEGDSGGPHVTEV
EGTSFLTGIISWGEECAIKGKYGVYTRVSWYVNWIKEKTKLT.
pTK1335 human FIX Padua cDNA
SEQ ID NO: 17
atgcagcgcgtgaacatgatcatggccgagagccctggcctgatcaccatctgcctgctgggctacctgc
tgagegccgagtgcaccgtgttcctggaccacgagaacgccaacaagatcctgaaccggcccaagagata
caacagcggcaagctggaggagttcgtgcagggcaacctggagagggagtgcatggaggagaagtgcagc
ttcgaggaggccagggaagtgttcgagaacaccgagcggaccaccgagttctggaagcagtacgtggacg
gcgaccagtgcgagagcaacccttgcctgaacggcggcagctgcaaggacgacatcaacagctacgagtg
ctggtgccctttcggcttcgagggcaagaactgcgagctggacgtgacctgcaacatcaagaacggccgc
tgcgagcagttctgcaagaacagcgccgacaacaaagtggtgtgtagctgcaccgagggctacagactgg
ccgagaaccagaagagctgcgagcccgccgtgcccttcccctgcggcagagtgagcgtgtcccagaccag
caagctgaccagagccgagaccgtgttccccgacgtggactacgtgaatagcaccgaggccgagaccatc
ctggacaacatcacccagagcacccagtccttcaacgacttcaccagagttgtgggcggcgaggacgcca
agcccggccagttcccctggcaggtggtgctgaacggcaaagtggatgccttctgcggcggcagcatcgt
gaacgagaagtggatcgtgacagccgcccactgcgtggagaccggcgtgaagatcaccgtggtggccggc
gaacacaatatcgaggagaccgagcacaccgagcagaagcggaacgtcatccggattatcccccaccaca
actacaacgccgccatcaacaagtacaaccacgacatcgccctgctggagctggacgagcctctggtgct
gaatagctacgtgacccccatctgcatcgccgacaaggagtacaccaacatcttcctgaagttcggcagc
ggctacgtgtccggctggggcagagtgttccacaagggcagaagcgccctggtgctgcagtacctgagag
tgcccctggtggacagagccacctgcctgTTGagcaccaagttcaccatctacaacaacatgttctgcgc
cggcttccacgagggcggcagagacagctgccagggcgacagcggcggaccccacgtgaccgaagtggag
ggcaccagcttcctgaccggcatcatcagctggggcgaggagtgcgccatgaagggcaagtacggcatct
acaccaaagtgagccggtacgtgaactggatcaaggagaaaaccaagctgacctga.
pTK1835 Chimeric human/rabbit Padua factor IX Padua cDNA
SEQ ID NO: 18
atgcagcgcgtgaacatgattatggccgagagccctggcctgatcaccatctgcctgctgggctacctgc
tgagcgccgagtgtaccgtgttcctggaccacgagaacgccaacaagatcctgaacagacccaagagata
caacagcggcaagctggaagagttcgtgcagggcaacctggaacgcgagtgcatggaagagaagtgcagc
ttcgaagaggccagagaggtgttcgagaacaccgagagaaccaccgagttctggaagcagtacgtggacg
gcgaccagtgcgagagcaacccttgtctgaacggcggcagctgcaaggacgacatcaacagctacgagtg
ctggtgccccttcggcttcgagggcaagaactgcgagctggacgtgacctgcaacatcaagaacggcaga
tgcgagcagttctgcaagaacagcgccgacaacaaggtcgtgtgctcctgcaccgagggctacagactgg
ccgagaaccagaagtcctgcgagcccgctgtgccctttccttgtggcggcgtgtccgtgtctcacgccag
caagaagatcaccagagccaccaccatcttcagcaacaccgagtacgagaacttcaccgaggccgagaca
atcagaggcaacgtgacccagagaagccagagcagcgacgacttcaccagaatcgtgggcggcgagaacg
ctaagcccggccagttcccgtggcaggtgctgctgaacggaaaggtggaagccttctgcggcggctccat
catcaacgagaaatgggtcgtgacagccgcccactgcatcaagcccgacgacaacatcaccgtggtggct
ggcgagtacaacatccaggaaaccgagaataccgaacagaaacggaacgtgatccgcatcatcccctacc
acaagtacaacgccacaatcaacaagtataaccacgatatcgccctgctggaactggacaagcccctgac
cctgaactcctacgtgacccccatctgtatcgccaacagagagtacaccaacatctttctgaacttcggc
agcggctacgtgtccggctggggcagagtgttcaacagaggcagacaggccagcatcctgcagtacctga
gagtgcccttcgtggacagggccacctgtctgCTGagcaccaagttcaccatctacaacaacatgttctg
cgctggcttcgatgtgggcggcaaggactcttgcgagggcgattctggcggccctcacgtgacagaggtg
gaaggcaccagctttctgaccggcatcatcagctggggcgaggaatgcgccatcaagggcaaatacggcg
tgtacaccagagtgtcttggtacgtgaactggatcaaagaaaagaccaagctgacctga.
pTK1847 Rabbit FIX Padua cDNA
SEQ ID NO: 19
atgcagtttctgaacaccatcatggccgagagccctggcctgatcaccatctgcctgctgggctacctgc
tgagcgccgagtgtaccgtgttcctggaccacgagaacgccacaaagatcctgaacagagccaagagata
caacagcggcaagctggaagagttcgtgtccggcaacctggaaagagagtgcatcgaggaaagatgcagc
ttcgaagaggccagagaggtgttcgagaacaccgaaaagaccaccgagttctggaagcagtacgtggacg
gcgaccagtgcgagagcaacccttgtctgaacggcggcagctgcaaggacgacatcaacgcctacgagtg
ctggtgccagtacggcttcgagggcaagaactgcgagctggacagcacctgtagcatcaagaacggcaga
tgcgagcagttctgccggaagaacagaaacaacaagatcatctgcagctgtaccgagggctacagactgg
ccgagaaccagaagtcctgcgagcccgctgtgcctttcccatgcggaagagtgtccgtgtctcacgccag
caagaagatcaccagagccaccaccatcttcagcaacaccgagtacgagaacttcaccgaggccgagaca
atcagaggcaacgtgacccaggacgcccagagcagcgacgacttcaccagaatcgtgggcggcgagaacg
ctaagcccggccagttcccgtggcaggtgctgctgaacggaaaggtggaagccttctgcggcggctccat
catcaacgagaaatgggtcgtgacagccgcccactgcatcaagcccgacgacaacatcacagtggtggcc
ggcgagtacaacatccaggaaaccgagaacacagagcagaaacggaacgtgatcaggatcatcccctacc
acaagtacaacgctaccatcaacaagtataaccacgatatcgccctgctggaactggacaagcccctgac
cctgaacagctacgtgacccccatctgtatcgccaacagagagtacaccaacatctttctgaacttcggc
agcggctacgtgtccggatggggcagagtgttcaacagaggcagacaggccagcatcctgcagtacctga
gagtgcccttcgtggacagggccacctgtctgCTGagcaccaagttcaccatctacaacaacatgttctg
cgccggcttcgatgtgggcggcaaggattcttgcgagggcgattctggcggccctcacgtgacagaggtg
gaaggcaccagctttctgaccggcatcatcagctggggcgaggaatgcgccatcaagggcaaatacggcg
tgtacaccagagtgtcttggtacgtgaactggatcaaagaaaagacaaagctgaccagcgtgtcccagac
cagcaagctgacaagagccgagacagtgttccccgatgtggacggctccgagcagaagctgatctccgaa
gaggacctggaacagaaactgatcagcgaggaagatctggaacagaagctgatttctgaagaagatctgg
aacagaaactgatctccgaagaggatctctga.
pTK1904 Rabbit FIX with human cleavage site α and Padua
SEQ ID NO: 20
atgcagtttctgaacaccatcatggccgagagccctggcctgatcaccatctgcctgctgggctacctgc
tgagcgccgagtgtaccgtgttcctggaccacgagaacgccacaaagatcctgaacagagccaagagata
caacagcggcaagctggaagagttcgtgtccggcaacctggaaagagagtgcatcgaggaaagatgcagc
ttcgaagaggccagagaggtgttcgagaacaccgaaaagaccaccgagttctggaagcagtacgtggacg
gcgaccagtgcgagagcaacccttgtctgaacggcggcagctgcaaggacgacatcaacgcctacgagtg
ctggtgccagtacggcttcgagggcaagaactgcgagctggacagcacctgtagcatcaagaacggcaga
tgcgagcagttctgccggaagaacagaaacaacaagatcatctgcagctgtaccgagggctacagactgg
ccgagaaccagaagtcctgcgagcccgctgtgcctttcccatgcggaagagtgtccgtgtcacaaacttc
taagctcacccgtgctgagactgtttttcctgatgtggactacgagaacttcaccgaggccgagacaatc
agaggcaacgtgacccaggacgcccagagcagcgacgacttcaccagaatcgtgggcggcgagaacgcta
agcccggccagttcccgtggcaggtgctgctgaacggaaaggtggaagccttctgcggcggctccatcat
caacgagaaatgggtcgtgacagccgcccactgcatcaagcccgacgacaacatcacagtggtggccggc
gagtacaacatccaggaaaccgagaacacagagcagaaacggaacgtgatcaggatcatcccctaccaca
agtacaacgctaccatcaacaagtataaccacgatatcgccctgctggaactggacaagcccctgaccct
gaacagctacgtgacccccatctgtatcgccaacagagagtacaccaacatctttctgaacttcggcagc
ggctacgtgtccggatggggcagagtgttcaacagaggcagacaggccagcatcctgcagtacctgagag
tgcccttcgtggacagggccacctgtctgCTGagcaccaagttcaccatctacaacaacatgttctgcgc
cggcttcgatgtgggcggcaaggattcttgcgagggcgattctggcggccctcacgtgacagaggtggaa
ggcaccagctttctgaccggcatcatcagctggggcgaggaatgcgccatcaagggcaaatacggcgtgt
acaccagagtgtcttggtacgtgaactggatcaaagaaaagacaaagctgaccagcgtgtcccagaccag
caagctgacaagagccgagacagtgttccccgatgtggacggctccgagcagaagctgatctccgaagag
gacctggaacagaaactgatcagcgaggaagatctggaacagaagctgatttctgaagaagatctggaac
agaaactgatctccgaagaggatctctga.
Rabbit FIX with human cleavage site β and Padua
SEQ ID NO: 21
atgcagtttctgaacaccatcatggccgagagccctggcctgatcaccatctgcctgctgggctacctgc
tgagcgccgagtgtaccgtgttcctggaccacgagaacgccacaaagatcctgaacagagccaagagata
caacagcggcaagctggaagagttcgtgtccggcaacctggaaagagagtgcatcgaggaaagatgcagc
ttcgaagaggccagagaggtgttcgagaacaccgaaaagaccaccgagttctggaagcagtacgtggacg
gcgaccagtgcgagagcaacccttgtctgaacggcggcagctgcaaggacgacatcaacgcctacgagtg
ctggtgccagtacggcttcgagggcaagaactgcgagctggacagcacctgtagcatcaagaacggcaga
tgcgagcagttctgccggaagaacagaaacaacaagatcatctgcagctgtaccgagggctacagactgg
ccgagaaccagaagtcctgcgagcccgctgtgcctttcccatgcggaagagtgtccgtgtctcacgccag
caagaagatcaccagagccaccaccatcttcagcaacaccgagtacgagaacttcaccgaggccgagaca
atcctggacaacatcacccagagcacccagtccttcaacgacttcaccagagttgtgggcggcgaggacg
ctaagcccggccagttcccgtggcaggtgctgctgaacggaaaggtggaagccttctgcggcggctccat
catcaacgagaaatgggtcgtgacagccgcccactgcatcaagcccgacgacaacatcacagtggtggcc
ggcgagtacaacatccaggaaaccgagaacacagagcagaaacggaacgtgatcaggatcatcccctacc
acaagtacaacgctaccatcaacaagtataaccacgatatcgccctgctggaactggacaagcccctgac
cctgaacagctacgtgacccccatctgtatcgccaacagagagtacaccaacatctttctgaacttcggc
agcggctacgtgtccggatggggcagagtgttcaacagaggcagacaggccagcatcctgcagtacctga
gagtgcccttcgtggacagggccacctgtctgCTGagcaccaagttcaccatctacaacaacatgttctg
cgccggcttcgatgtgggcggcaaggattcttgcgagggcgattctggcggccctcacgtgacagaggtg
gaaggcaccagctttctgaccggcatcatcagctggggcgaggaatgcgccatcaagggcaaatacggcg
tgtacaccagagtgtcttggtacgtgaactggatcaaagaaaagacaaagctgaccagcgtgtcccagac
cagcaagctgacaagagccgagacagtgttccccgatgtggacggctccgagcagaagctgatctccgaa
gaggacctggaacagaaactgatcagcgaggaagatctggaacagaagctgatttctgaagaagatctgg
aacagaaactgatctccgaagaggatctctga.
Rabbit FIX Rabbit FIX with human cleavage site
α and cleavage site Band Padua
SEQ ID NO: 22
atgcagtttctgaacaccatcatggccgagagccctggcctgatcaccatctgcctgctgggctacctgc
tgagcgccgagtgtaccgtgttcctggaccacgagaacgccacaaagatcctgaacagagccaagagata
caacagcggcaagctggaagagttcgtgtccggcaacctggaaagagagtgcatcgaggaaagatgcagc
ttcgaagaggccagagaggtgttcgagaacaccgaaaagaccaccgagttctggaagcagtacgtggacg
gcgaccagtgcgagagcaacccttgtctgaacggcggcagctgcaaggacgacatcaacgcctacgagtg
ctggtgccagtacggcttcgagggcaagaactgcgagctggacagcacctgtagcatcaagaacggcaga
tgcgagcagttctgccggaagaacagaaacaacaagatcatctgcagctgtaccgagggctacagactgg
ccgagaaccagaagtcctgcgagcccgctgtgcctttcccatgcggaagagtgtccgtgtcacaaacttc
taagctcacccgtgctgagactgtttttcctgatgtggactacgagaacttcaccgaggccgagacaatc
ctggacaacatcacccagagcacccagtccttcaacgacttcaccagagttgtgggcggcgaggacgcta
agcccggccagttcccgtggcaggtgctgctgaacggaaaggtggaagccttctgcggcggctccatcat
caacgagaaatgggtcgtgacagccgcccactgcatcaagcccgacgacaacatcacagtggtggccggc
gagtacaacatccaggaaaccgagaacacagagcagaaacggaacgtgatcaggatcatcccctaccaca
agtacaacgctaccatcaacaagtataaccacgatatcgccctgctggaactggacaagcccctgaccct
gaacagctacgtgacccccatctgtatcgccaacagagagtacaccaacatctttctgaacttcggcagc
ggctacgtgtccggatggggcagagtgttcaacagaggcagacaggccagcatcctgcagtacctgagag
tgcccttcgtggacagggccacctgtctgCTGagcaccaagttcaccatctacaacaacatgttctgcgc
cggcttcgatgtgggcggcaaggattcttgcgagggcgattctggcggccctcacgtgacagaggtggaa
ggcaccagctttctgaccggcatcatcagctggggcgaggaatgcgccatcaagggcaaatacggcgtgt
acaccagagtgtcttggtacgtgaactggatcaaagaaaagacaaagctgaccagcgtgtcccagaccag
caagctgacaagagccgagacagtgttccccgatgtggacggctccgagcagaagctgatctccgaagag
gacctggaacagaaactgatcagcgaggaagatctggaacagaagctgatttctgaagaagatctggaac
agaaactgatctccgaagaggatctctga.
Rabbit FIX Rabbit FIX with human Cleavage site
α and Cleavage siteb and human active peptide
Padua
SEQ ID NO: 23
atgcagtttctgaacaccatcatggccgagagccctggcctgatcaccatctgcctgctgggctacctgc
tgagcgccgagtgtaccgtgttcctggaccacgagaacgccacaaagatcctgaacagagccaagagata
caacagcggcaagctggaagagttcgtgtccggcaacctggaaagagagtgcatcgaggaaagatgcagc
ttcgaagaggccagagaggtgttcgagaacaccgaaaagaccaccgagttctggaagcagtacgtggacg
gcgaccagtgcgagagcaacccttgtctgaacggcggcagctgcaaggacgacatcaacgcctacgagtg
ctggtgccagtacggcttcgagggcaagaactgcgagctggacagcacctgtagcatcaagaacggcaga
tgcgagcagttctgccggaagaacagaaacaacaagatcatctgcagctgtaccgagggctacagactgg
ccgagaaccagaagtcctgcgagcccgctgtgcctttcccatgcggaagagtgtccgtgtcacaaacttc
taagctcacccgtgctgagactgtttttcctgatgtggactacgtgaacagcaccgaggccgagacaatc
ctggacaacatcacccagagcacccagtccttcaacgacttcaccagagttgtgggcggcgaggacgcta
agcccggccagttcccgtggcaggtgctgctgaacggaaaggtggaagccttctgcggcggctccatcat
caacgagaaatgggtcgtgacagccgcccactgcatcaagcccgacgacaacatcacagtggtggccggc
gagtacaacatccaggaaaccgagaacacagagcagaaacggaacgtgatcaggatcatcccctaccaca
agtacaacgctaccatcaacaagtataaccacgatatcgccctgctggaactggacaagcccctgaccct
gaacagctacgtgacccccatctgtatcgccaacagagagtacaccaacatctttctgaacttcggcagc
ggctacgtgtccggatggggcagagtgttcaacagaggcagacaggccagcatcctgcagtacctgagag
tgcccttcgtggacagggccacctgtctgCTGagcaccaagttcaccatctacaacaacatgttctgcgc
cggcttcgatgtgggcggcaaggattcttgcgagggcgattctggcggccctcacgtgacagaggtggaa
ggcaccagctttctgaccggcatcatcagctggggcgaggaatgcgccatcaagggcaaatacggcgtgt
acaccagagtgtcttggtacgtgaactggatcaaagaaaagacaaagctgaccagcgtgtcccagaccag
caagctgacaagagccgagacagtgttccccgatgtggacggctccgagcagaagctgatctccgaagag
gacctggaacagaaactgatcagcgaggaagatctggaacagaagctgatttctgaagaagatctggaac
agaaactgatctccgaagaggatctctga.
GenBank® Accession No. AAA56822.1; complete
SEQ ID NO: 24
MQRVNMIMAESPGLITICLLGYLLSAECTVELDHENANKILNRPKRYNSGKLEEFVOGNLERECMEEKCS
FEEAREVFENTERTTEFWKQYVDGDQCESNPCLNGGSCKDDINSYECWCPFGFEGKNCELDVTCNIKNGR
CEQFCKNSADNKVVCSCTEGYRLAENQKSCEPAVPFPCGRVSVSQTSKLTRAEAVFPDVDYVNSTEAETI
LDNITQSTQSFNDFTRVVGGEDAKPGQFPWQVVLNGKVDAFCGGSIVNEKWIVTAAHCVETGVKITVVAG
EHNIEETEHTEQKRNVIRIIPHHNYNAAINKYNHDIALLELDEPLVLNSYVTPICIADKEYTNIFLKFGS
GYVSGWGRVFHKGRSALVLQYLRVPLVDRATCLRSTKFTIYNNMFCAGFHEGGRDSCQGDSGGPHVTEVE
GTSFLTGIISWGEECAMKGKYGIYTKVSRYVNWIKEKTKLT.
GenBank® Accession NO. QWM97845.1; comprising Padua.
SEQ ID NO: 25
MQRVNMIMAESPGLITICLLGYLLSAECTVELDHENANKILNRPKRYNSGKLEEFVQGNLERECMEEKCS
FEEAREVFENTERTTEFWKQYVDGDQCESNPCLNGGSCKDDINSYECWCPFGFEGKNCELDVTCNIKNGR
CEQFCKNSADNKVVCSCTEGYRLAENQKSCEPAVPFPCGRVSVSQTSKLTRAETVFPDVDYVNSTEAETI
LDNITQSTQSFNDFTRVVGGEDAKPGQFPWQVVLNGKVDAFCGGSIVNEKWIVTAAHCVETGVKITVVAG
EHNIEETEHTEQKRNVIRIIPHHNYNAAINKYNHDIALLELDEPLVLNSYVTPICIADKEYTNIFLKFGS
GYVSGWGRVFHKGRSALVLQYLRVPLVDRATCLLSTKFTIYNNMFCAGFHEGGRDSCQGDSGGPHVTLVE
GTWFLTGIISWGEECAMKGKYGIYTKVSRYVNWIKEKTKLT.
cTK2294 (depicted in FIG. 6) encoding a fusion
protein comprisingaptamer resistant factor IX
and a tandem of Fc regions.
SEQ ID NO: 26
atgcagtttctgaacaccatcatggccgagagccctggcctgatcaccatctgcctgctgggctacctgc
tgagcgccgagtgtaccgtgttcctggaccacgagaacgccacaaagatcctgaacagagccaagagata
caacagcggcaagctggaagagttcgtgtccggcaacctggaaagagagtgcatcgaggaaagatgcagc
ttcgaagaggccagagaggtgttcgagaacaccgaaaagaccaccgagttctggaagcagtacgtggacg
gcgaccagtgcgagagcaacccttgtctgaacggcggcagctgcaaggacgacatcaacgcctacgagtg
ctggtgccagtacggcttcgagggcaagaactgcgagctggacagcacctgtagcatcaagaacggcaga
tgcgagcagttctgccggaagaacagaaacaacaagatcatctgcagctgtaccgagggctacagactgg
ccgagaaccagaagtcctgcgagcccgctgtgcctttcccatgcggaagagtgtccgtgtcacaaacttc
taagctcacccgtgctgagactgtttttcctgatgtggactacgagaacttcaccgaggccgagacaatc
agaggcaacgtgacccaggacgcccagagcagcgacgacttcaccagaatcgtgggcggcgagaacgcta
agcccggccagttcccgtggcaggtgctgctgaacggaaaggtggaagccttctgcggcggctccatcat
caacgagaaatgggtcgtgacagccgcccactgcatcaagcccgacgacaacatcacagtggtggccggc
gagtacaacatccaggaaaccgagaacacagagcagaaacggaacgtgatcaggatcatcccctaccaca
agtacaacgctaccatcaacaagtataaccacgatatcgccctgctggaactggacaagcccctgaccct
gaacagctacgtgacccccatctgtatcgccaacagagagtacaccaacatctttctgaacttcggcagc
ggctacgtgtccggatggggcagagtgttcaacagaggcagacaggccagcatcctgcagtacctgagag
tgcccttcgtggacagggccacctgtctgctgagcaccaagttcaccatctacaacaacatgttctgcgc
cggcttcgatgtgggggcaaggattcttgcgagggcgattctggcggccctcacgtgacagaggtggaag
gcaccagctttctgaccggcatcatcagctggggcgaggaatgcgccatcaagggcaaatacggcgtgta
caccagagtgtcttggtacgtgaactggatcaaagaaaagacaaagctgaccagcgtgtcccagaccagc
aagctgacaagagccgagacagtgttccccgatgtggacggctccgagcagaagctgatctccgaagagg
acctggaacagaaactgatcagcgaggaagatctggaacagaagctgatttctgaagaagatctggaaca
gaaactgatctccgaagaggatctcGGCAGCGACAAGACCCACACCTGTCCTCCATGTCCCGCCCCTGAA
CTGCTGGGCGGACCTAGCGTGTTCCTGTTCCCCCCAAAGCCCAAGGACACCCTGATGATCAGCCGGACCC
CCGAAGTGACCTGCGTGGTGGTGGATGTGTCCCACGAGGACCCTGAAGTGAAGTTCAATTGGTATGTGGA
TGGCGTGGAAGTGCACAACGCCAAGACAAAGCCCAGAGAGGAACAGTACAACTCCACCTACCGGGTGGTG
TCCGTGCTGACCGTGCTGCACCAGGACTGGCTGAATGGCAAAGAGTATAAGTGCAAAGTGTCCAACAAGG
CCCTGCCTGCCCCCATCGAGAAAACCATCAGCAAGGCCAAGGGCCAGCCCCGCGAACCCCAGGTGTACAC
ACTGCCCCCTAGCAGGGACGAGCTGACCAAGAACCAGGTGTCCCTGACCTGTCTCGTGAAGGGCTTCTAC
CCTAGCCGGCTGGCCGTGGAATGGGAGAGCAATGGCCAGCCCGAGAACAATTACAAGACCACCCCCCCTG
TGCTGGACAGCGACGGCTCATTCTTCCTGTACAGCAAACTGACCGTGGACAAGAGCCGGTGGCAGCAGGG
CAATGTGTTCAGCTGTAGCGTGATGCACGAGGCCCTGCACAACCACTACACCCAGAAGTCTCTGAGCCTG
AGCCCCGGCAAGGGCGGAGGAAGTGGGGGAGGATCTGGCGGCGGCTCCGATAAGACACATACCTGCCCCC
CTTGCCCTGCCCCAGAGCTGCTGGGAGGCCCTTCTGTGTTTCTGTTTCCACCTAAGCCTAAAGATACACT
GATGATCTCCCGCACACCTGAAGTGACATGTGTGGTGGTGGACGTGTCACATGAAGATCCAGAAGTGAAG
TTTAATTGGTACGTGGACGGGGTGGAAGTGCATAATGCTAAGACCAAACCCCGGGAAGAACAGTATAACA
GCACATACAGAGTGGTGTCTGTGCTGACAGTGCTGCATCAGGATTGGCTGAACGGGAAAGAATACAAATG
TAAAGTGTCTAACAAAGCTCTGCCCGCTCCTATCGAAAAGACAATCTCCAAGGCTAAAGGACAGCCCAGA
GAACCTCAGGTGTACACACTGCCTCCATCCCGCGACGAGCTGACAAAAAATCAGGTGTCACTGACATGCC
TCGTGAAGGGGTTTTATCCATCTAGGCTGGCTGTGGAATGGGAATCCAACGGACAGCCTGAAAACAACTA
TAAGACAACACCTCCCGTGCTGGACTCCGATGGCTCATTTTTTCTGTATTCCAAGCTGACTGTGGATAAG
TCCAGATGGCAGCAGGGAAACGTGTTCTCCTGTTCTGTGATGCATGAAGCTCTGCATAATCATTATACCC
AGAAAAGCCTGTCCCTGTCCCCTGGCAAGTGA.
AA sequence for FIX portion of novel molecule
linked to FC, encodedby cDNA cTK2294
SEQ ID NO: 27
MQFLNTIMAESPGLITICLLGYLLSAECTVFLDHENATKILNRAKRYNSGKLEEFVSGNLERECIEERCS
FEEAREVFENTEKTTEFWKQYVDGDQCESNPCLNGGSCKDDINAYECWCQYGFEGKNCELDSTCSIKNGR
CEQFCRKNRNNKIICSCTEGYRLAENQKSCEPAVPFPCGRVSVSQTSKLTRAETVFPDVDYENFTEAETI
RGNVTQDAQSSDDFTRIVGGENAKPGQFPWQVLLNGKVEAFCGGSIINEKWVVTAAHCIKPDDNITVVAG
EYNIQETENTEQKRNVIRIIPYHKYNATINKYNHDIALLELDKPLTLNSYVTPICIANREYTNIFLNFGS
GYVSGWGRVFNRGRQASILQYLRVPFVDRATCLLSTKFTIYNNMFCAGFDVGGKDSCEGDSGGPHVTEVE
GTSFLTGIISWGEECAIKGKYGVYTRVSWYVNWIKEKTKLTSVSQTSKLTRAETVFPDVDGSEQKLISEE
DLEQKLISEEDLEQKLISEEDLEQKLISEEDLGSDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSRLAVEWESNGQPENNYKTTPP
VLDSDGSFFLYSKLTVDKSRWQQGNVFSCSVMHEALHNHYTQKSLSLSPGKGGGSGGGSGGGSDKTHTCP
PCPAPELLGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDELTKNQVSLTC
LVKGFYPSRLAVEWESNGQPENNYKTTPPVLDSDGSFFLYSKLTVDKSRWQQGNVFSCSVMHEALHNHYT
QKSLSLSPGK.

The invention claimed is:

1-46. (canceled)

47. A recombinant Factor IX protein of a mammal, comprising:
(a) one or more substitution(s) of an XIa cleavage site-α and/or an XIa cleavage site-β of a human Factor IX protein; and/or
(b) an N-terminal substitution of a human Factor IX N-terminus amino acid segment;
wherein the recombinant Factor IX protein is resistant to inhibition by aptamer 9.3t.

48. (canceled)

49. The recombinant Factor IX protein of claim 47, further comprising substitution of one or more neighboring amino acid residues of the human XIa cleavage site-α and/or the human XIa cleavage site-β.

50. The recombinant Factor IX protein of claim 47, wherein the substitution of a human Factor IX N-terminus amino acid segment comprises at least 100 N-terminal amino acid residues of said human Factor IX protein.

51. The recombinant Factor IX protein of claim 47, further comprising a leucine substitution at position 385 wherein the numbering corresponds to SEQ ID NO:2.

52. (canceled)

53. The recombinant Factor IX protein of claim 47, wherein the substitution of an XIa cleavage site-α comprises a substitution of amino acid residue positions 182-201 and/or an insertion at the C-terminus corresponding to SEQ ID NO:2.

54. The recombinant Factor IX protein of claim 47, wherein the substitution of an XIa cleavage site-α comprises the amino acid sequence SVSQTSKLTRAETVFPDVD (SEQ ID NO: 7).

55. The recombinant Factor IX protein of claim 47, wherein the substitution of an XIa cleavage site-β comprises a substitution of amino acid residue positions 212-233 corresponding to SEQ ID NO:2.

56. The recombinant Factor IX protein of claim 47, wherein the substitution of an XIa cleavage site-β comprises the amino acid sequence LDNITQSTQSFNDFTRVVGGED (SEQ ID NO:8).

57-58. (canceled)

59. The recombinant Factor IX protein of claim 47, comprising the amino acid sequence of SEQ ID NO: 12 or an amino acid sequence at least 90% identical thereto.

60. The recombinant Factor IX protein of claim 47, comprising the amino acid sequence of any one of SEQ ID NOs: 13-16 or 27 or an amino acid sequence at least 90% identical thereto.

61-64. (canceled)

65. An isolated nucleic acid molecule encoding the recombinant Factor IX protein of claim 47.

66. (canceled)

67. The isolated nucleic acid molecule of claim 65, comprising the nucleotide sequence of any one of SEQ ID NOs: 17-23 or 26.

68. A vector comprising the nucleic acid molecule of claim 65.

69. A transformed cell comprising the nucleic acid molecule of claim 65.

70. (canceled)

71. A composition comprising the recombinant Factor IX protein of claim 47.

72. (canceled)

73. A method of identifying a Factor IX protein as resistant to inhibition by aptamer 9.3t, comprising:
(i) introducing an effective amount of aptamer 9.3t to one or more sample(s) comprising an endogenous Factor IX protein of a mammalian subject, and then assaying for the presence of clotting activity in the one or more sample(s) comprising said effective amount of aptamer 9.3t, wherein the presence of clotting activity in the one or more sample(s) comprising aptamer 9.3t indicates resistance of the endogenous Factor IX to inhibition by aptamer 9.3t and wherein the lack of clotting activity in the one or more sample(s) comprising aptamer 9.3t indicates sensitivity of the endogenous Factor IX to inhibition by aptamer 9.3t, as compared to a control sample; and
(ii) identifying the endogenous Factor IX comprised in the one or more sample(s) comprising an effective amount of aptamer 9.3t as sensitive to aptamer 9.3t or identifying the endogenous Factor X comprised in the one or more sample(s) unable to determine an effective amount of aptamer 9.3t as resistant to aptamer 9.3t.

74-75. (canceled)

76. A recombinant Factor IX protein identified as resistant to aptamer 9.3t by the method of claim 73.

77-84. (canceled)

85. An in vitro method of modeling protein and/or gene replacement therapy for treating a bleeding disorder in one or more non-hemophilic subject, comprising:
(i) providing a sample from one or more protein non-deficient subject comprising an endogenous Factor IX protein;
(ii) contacting the sample with an effective amount of aptamer 9.3t, wherein the effective amount of the aptamer reduces activity of the endogenous Factor IX protein; and
(iii) contacting the sample with an effective amount of the exogenous aptamer-resistant replacement Factor IX protein of claim 47.

86. An in vitro method of identifying genetic background effects of one or more non-hemophilic subject on an exogenous Factor IX protein, comprising the steps of:
(i) providing a sample from one or more non-hemophilic subject comprising an endogenous Factor IX protein;
(ii) contacting the sample with an effective amount of aptamer 9.3t, wherein the effective amount of the aptamer 9.3t reduces activity of the endogenous Factor IX protein;
(iii) contacting the sample with an effective amount of the exogenous aptamer-resistant replacement Factor IX protein of claim 47;
(iv) assaying for the presence of protein clotting activity in the sample comprising the exogenous aptamer-resistant replacement Factor IX protein and in a control;
(v) comparing the relative level and/or presence of protein clotting activity of the sample comprising the exogenous aptamer-resistant replacement Factor IX protein and the control to identify genetic background effects of the subject on the efficacy and/or safety of the exogenous replacement Factor IX protein; and
(vi) associating the difference in relative level and/or presence of the protein clotting activity with genetic background differences between the one or more subject and the control.

87-88. (canceled)

89. A method of modeling a Factor IX protein and/or gene replacement therapy for treating a bleeding disorder in one or more non-hemophilic subject, comprising:
(i) administering to the one or more subject an effective amount of aptamer 9.3t, wherein the effective amount of aptamer 9.3t reduces activity of endogenous Factor IX protein;
(ii) administering to the one or more subject an effective amount of the Factor IX protein of claim 47.

90-91. (canceled)

92. A method of identifying genetic background effects on an exogenous Factor IX protein in one or more non-hemophilic subject, comprising the steps of:
(i) administering to the one or more subject an effective amount of aptamer 9.3t, wherein the effective amount of aptamer 9.3t reduces activity of endogenous Factor IX protein;
(ii) administering to the one or more subject an effective amount of the Factor IX protein of claim 47;
(iii) assaying for the presence of clotting activity in the subject comprising the exogenous Factor IX protein and in a control;
(iv) comparing the relative presence of clotting activity of the one or more subject comprising the exogenous Factor IX protein and the control to identify genetic background effects of the subject on the efficacy of the exogenous Factor IX protein; and
(v) associating the difference in relative level and/or presence of the clotting activity with genetic background differences between the one or more subject and the control.

93-102. (canceled)


SEQUENCE LISTING (27 sequences)

SEQ ID NO: 1 - protein, 415 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAEAVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 2 - protein, 462 aa, Oryctolagus cuniculus
MQFLNTIMAESPGLITICLLGYLLSAECTVFLDHENATKILNRAKRYNSGKLEEFVSGNLERECIEERCSFEEAREVFENTEKTTEFWKQYVDGDQCESNPCLNGGSCKDDINAYECWCQYGFEGKNCELDSTCSIKNGRCEQFCRKNRNNKIICSCTEGYRLAENQKSCEPAVPFPCGRVSVSHASKKITRATTIFSNTEYENFTEAETIRGNVTQDAQSSDDFTRIVGGENAKPGQFPWQVLLNGKVEAFCGGSIINEKWVVTAAHCIKPDDNITVVAGEYNIQETENTEQKRNVIRIIPYHKYNATINKYNHDIALLELDKPLTLNSYVTPICIANREYTNIFLNFGSGYVSGWGRVFNRGRQASILQYLRVPFVDRATCLRSTKFTIYNNMFCAGFDVGGKDSCEGDSGGPHVTEVEGTSFLTGIISWGEECAIKGKYGVYTRVSWYVNWIKEKTKLT

SEQ ID NO: 3 - protein, 20 aa, Oryctolagus cuniculus
SVSHASKKITRATTIFSNTE

SEQ ID NO: 4 - protein, 22 aa, Oryctolagus cuniculus
RGNVTQDAQSSDDFTRIVGGEN

SEQ ID NO: 5 - protein, 35 aa, Oryctolagus cuniculus
ATTIFSNTEYENFTEAETIRGNVTQDAQSSDDFTR

SEQ ID NO: 6 - protein, 52 aa, Oryctolagus cuniculus
SVSHASKKITRATTIFSNTEYENFTEAETIRGNVTQDAQSSDDFTRIVGGEN

SEQ ID NO: 7 - protein, 19 aa, Homo sapiens
SVSQTSKLTRAETVFPDVD

SEQ ID NO: 8 - protein, 22 aa, Homo sapiens
LDNITQSTQSFNDFTRVVGGED

SEQ ID NO: 9 - protein, 35 aa, Homo sapiens
AETVFPDVDYVNSTEAETILDNITQSTQSFNDFTR

SEQ ID NO: 10 - protein, 51 aa, Homo sapiens
SVSQTSKLTRAETVFPDVDYVNSTEAETILDNITQSTQSFNDFTRVVGGED

SEQ ID NO: 11 - protein, 40 aa, synthetic construct
  source 1..40: note = myc tag
EQKLISEEDLEQKLISEEDLEQKLISEEDLEQKLISEEDL

SEQ ID NO: 12 - protein, 522 aa, synthetic construct
  source 1..522: note = Modified Rabbit Padua FIX comprising human cleavage site alpha, human activation peptide, and human cleavage site beta and additional human cleavage site alpha and myc tag at end
MQFLNTIMAESPGLITICLLGYLLSAECTVFLDHENATKILNRAKRYNSGKLEEFVSGNLERECIEERCSFEEAREVFENTEKTTEFWKQYVDGDQCESNPCLNGGSCKDDINAYECWCQYGFEGKNCELDSTCSIKNGRCEQFCRKNRNNKIICSCTEGYRLAENQKSCEPAVPFPCGRVSVSQTSKLTRAETVFPDVDYENFTEAETILDNITQSTQSFNDFTRVVGGEDAKPGQFPWQVLLNGKVEAFCGGSIINEKWVVTAAHCIKPDDNITVVAGEYNIQETENTEQKRNVIRIIPYHKYNATINKYNHDIALLELDKPLTLNSYVTPICIANREYTNIFLNFGSGYVSGWGRVFNRGRQASILQYLRVPFVDRATCLLSTKFTIYNNMFCAGFDVGGKDSCEGDSGGPHVTEVEGTSFLTGIISWGEECAIKGKYGVYTRVSWYVNWIKEKTKLTSVSQTSKLTRAETVFPDVDGSEQKLISEEDLEQKLISEEDLEQKLISEEDLEQKLISEEDL

SEQ ID NO: 13 - protein, 522 aa, synthetic construct
  source 1..522: note = Modified Rabbit Padua FIX
MQFLNTIMAESPGLITICLLGYLLSAECTVFLDHENATKILNRAKRYNSGKLEEFVSGNLERECIEERCSFEEAREVFENTEKTTEFWKQYVDGDQCESNPCLNGGSCKDDINAYECWCQYGFEGKNCELDSTCSIKNGRCEQFCRKNRNNKIICSCTEGYRLAENQKSCEPAVPFPCGRVSVSQTSKLTRAETVFPDVDYENFTEAETIRGNVTQDAQSSDDFTRIVGGENAKPGQFPWQVLLNGKVEAFCGGSIINEKWVVTAAHCIKPDDNITVVAGEYNIQETENTEQKRNVIRIIPYHKYNATINKYNHDIALLELDKPLTLNSYVTPICIANREYTNIFLNFGSGYVSGWGRVFNRGRQASILQYLRVPFVDRATCLLSTKFTIYNNMFCAGFDVGGKDSCEGDSGGPHVTEVEGTSFLTGIISWGEECAIKGKYGVYTRVSWYVNWIKEKTKLTSVSQTSKLTRAETVFPDVDGSEQKLISEEDLEQKLISEEDLEQKLISEEDLEQKLISEEDL

SEQ ID NO: 14 - protein, 523 aa, synthetic construct
  source 1..523: note = Modified Rabbit Padua FIX
MQFLNTIMAESPGLITICLLGYLLSAECTVFLDHENATKILNRAKRYNSGKLEEFVSGNLERECIEERCSFEEAREVFENTEKTTEFWKQYVDGDQCESNPCLNGGSCKDDINAYECWCQYGFEGKNCELDSTCSIKNGRCEQFCRKNRNNKIICSCTEGYRLAENQKSCEPAVPFPCGRVSVSHASKKITRATTIFSNTEYENFTEAETILDNITQSTQSFNDFTRVVGGEDAKPGQFPWQVLLNGKVEAFCGGSIINEKWVVTAAHCIKPDDNITVVAGEYNIQETENTEQKRNVIRIIPYHKYNATINKYNHDIALLELDKPLTLNSYVTPICIANREYTNIFLNFGSGYVSGWGRVFNRGRQASILQYLRVPFVDRATCLLSTKFTIYNNMFCAGFDVGGKDSCEGDSGGPHVTEVEGTSFLTGIISWGEECAIKGKYGVYTRVSWYVNWIKEKTKLTSVSQTSKLTRAETVFPDVDGSEQKLISEEDLEQKLISEEDLEQKLISEEDLEQKLISEEDL

SEQ ID NO: 15 - protein, 523 aa, synthetic construct
  source 1..523: note = Modified Rabbit Padua FIX
MQFLNTIMAESPGLITICLLGYLLSAECTVFLDHENATKILNRAKRYNSGKLEEFVSGNLERECIEERCSFEEAREVFENTEKTTEFWKQYVDGDQCESNPCLNGGSCKDDINAYECWCQYGFEGKNCELDSTCSIKNGRCEQFCRKNRNNKIICSCTEGYRLAENQKSCEPAVPFPCGRVSVSHASKKITRATTIFSNTEYENFTEAETIRGNVTQDAQSSDDFTRIVGGENAKPGQFPWQVLLNGKVEAFCGGSIINEKWVVTAAHCIKPDDNITVVAGEYNIQETENTEQKRNVIRIIPYHKYNATINKYNHDIALLELDKPLTLNSYVTPICIANREYTNIFLNFGSGYVSGWGRVFNRGRQASILQYLRVPFVDRATCLLSTKFTIYNNMFCAGFDVGGKDSCEGDSGGPHVTEVEGTSFLTGIISWGEECAIKGKYGVYTRVSWYVNWIKEKTKLTSVSQTSKLTRAETVFPDVDGSEQKLISEEDLEQKLISEEDLEQKLISEEDLEQKLISEEDL

SEQ ID NO: 16 - protein, 462 aa, synthetic construct
  source 1..462: note = Chimeric Human/Rabbit Padua FIX
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGGVSVSHASKKITRATTIFSNTEYENFTEAETIRGNVTQRSQSSDDFTRIVGGENAKPGQFPWQVLLNGKVEAFCGGSIINEKWVVTAAHCIKPDDNITVVAGEYNIQETENTEQKRNVIRIIPYHKYNATINKYNHDIALLELDKPLTLNSYVTPICIANREYTNIFLNFGSGYVSGWGRVFNRGRQASILQYLRVPFVDRATCLLSTKFTIYNNMFCAGFDVGGKDSCEGDSGGPHVTEVEGTSFLTGIISWGEECAIKGKYGVYTRVSWYVNWIKEKTKLT

SEQ ID NO: 17 - DNA, 1386 bp, synthetic construct
  source 1..1386: mol_type = other DNA; note = pTK1335 human FIX Padua cDNA
atgcagcgcgtgaacatgatcatggccgagagccctggcctgatcaccatctgcctgctgggctacctgctgagcgccgagtgcaccgtgttcctggaccacgagaacgccaacaagatcctgaaccggcccaagagatacaacagcggcaagctggaggagttcgtgcagggcaacctggagagggagtgcatggaggagaagtgcagcttcgaggaggccagggaagtgttcgagaacaccgagcggaccaccgagttctggaagcagtacgtggacggcgaccagtgcgagagcaacccttgcctgaacggcggcagctgcaaggacgacatcaacagctacgagtgctggtgccctttcggcttcgagggcaagaactgcgagctggacgtgacctgcaacatcaagaacggccgctgcgagcagttctgcaagaacagcgccgacaacaaagtggtgtgtagctgcaccgagggctacagactggccgagaaccagaagagctgcgagcccgccgtgcccttcccctgcggcagagtgagcgtgtcccagaccagcaagctgaccagagccgagaccgtgttccccgacgtggactacgtgaatagcaccgaggccgagaccatcctggacaacatcacccagagcacccagtccttcaacgacttcaccagagttgtgggcggcgaggacgccaagcccggccagttcccctggcaggtggtgctgaacggcaaagtggatgccttctgcggcggcagcatcgtgaacgagaagtggatcgtgacagccgcccactgcgtggagaccggcgtgaagatcaccgtggtggccggcgaacacaatatcgaggagaccgagcacaccgagcagaagcggaacgtcatccggattatcccccaccacaactacaacgccgccatcaacaagtacaaccacgacatcgccctgctggagctggacgagcctctggtgctgaatagctacgtgacccccatctgcatcgccgacaaggagtacaccaacatcttcctgaagttcggcagcggctacgtgtccggctggggcagagtgttccacaagggcagaagcgccctggtgctgcagtacctgagagtgcccctggtggacagagccacctgcctgttgagcaccaagttcaccatctacaacaacatgttctgcgccggcttccacgagggcggcagagacagctgccagggcgacagcggcggaccccacgtgaccgaagtggagggcaccagcttcctgaccggcatcatcagctggggcgaggagtgcgccatgaagggcaagtacggcatctacaccaaagtgagccggtacgtgaactggatcaaggagaaaaccaagctgacctga

SEQ ID NO: 18 - DNA, 1389 bp, synthetic construct
  source 1..1389: mol_type = other DNA; note = pTK1835 Chimeric human/rabbit Padua factor IX Padua cDNA
atgcagcgcgtgaacatgattatggccgagagccctggcctgatcaccatctgcctgctgggctacctgctgagcgccgagtgtaccgtgttcctggaccacgagaacgccaacaagatcctgaacagacccaagagatacaacagcggcaagctggaagagttcgtgcagggcaacctggaacgcgagtgcatggaagagaagtgcagcttcgaagaggccagagaggtgttcgagaacaccgagagaaccaccgagttctggaagcagtacgtggacggcgaccagtgcgagagcaacccttgtctgaacggcggcagctgcaaggacgacatcaacagctacgagtgctggtgccccttcggcttcgagggcaagaactgcgagctggacgtgacctgcaacatcaagaacggcagatgcgagcagttctgcaagaacagcgccgacaacaaggtcgtgtgctcctgcaccgagggctacagactggccgagaaccagaagtcctgcgagcccgctgtgccctttccttgtggcggcgtgtccgtgtctcacgccagcaagaagatcaccagagccaccaccatcttcagcaacaccgagtacgagaacttcaccgaggccgagacaatcagaggcaacgtgacccagagaagccagagcagcgacgacttcaccagaatcgtgggcggcgagaacgctaagcccggccagttcccgtggcaggtgctgctgaacggaaaggtggaagccttctgcggcggctccatcatcaacgagaaatgggtcgtgacagccgcccactgcatcaagcccgacgacaacatcaccgtggtggctggcgagtacaacatccaggaaaccgagaataccgaacagaaacggaacgtgatccgcatcatcccctaccacaagtacaacgccacaatcaacaagtataaccacgatatcgccctgctggaactggacaagcccctgaccctgaactcctacgtgacccccatctgtatcgccaacagagagtacaccaacatctttctgaacttcggcagcggctacgtgtccggctggggcagagtgttcaacagaggcagacaggccagcatcctgcagtacctgagagtgcccttcgtggacagggccacctgtctgctgagcaccaagttcaccatctacaacaacatgttctgcgctggcttcgatgtgggcggcaaggactcttgcgagggcgattctggcggccctcacgtgacagaggtggaaggcaccagctttctgaccggcatcatcagctggggcgaggaatgcgccatcaagggcaaatacggcgtgtacaccagagtgtcttggtacgtgaactggatcaaagaaaagaccaagctgacctga

SEQ ID NO: 19 - DNA, 1572 bp, synthetic construct
  source 1..1572: mol_type = other DNA; note = pTK1847 Rabbit FIX Padua cDNA
atgcagtttctgaacaccatcatggccgagagccctggcctgatcaccatctgcctgctgggctacctgctgagcgccgagtgtaccgtgttcctggaccacgagaacgccacaaagatcctgaacagagccaagagatacaacagcggcaagctggaagagttcgtgtccggcaacctggaaagagagtgcatcgaggaaagatgcagcttcgaagaggccagagaggtgttcgagaacaccgaaaagaccaccgagttctggaagcagtacgtggacggcgaccagtgcgagagcaacccttgtctgaacggcggcagctgcaaggacgacatcaacgcctacgagtgctggtgccagtacggcttcgagggcaagaactgcgagctggacagcacctgtagcatcaagaacggcagatgcgagcagttctgccggaagaacagaaacaacaagatcatctgcagctgtaccgagggctacagactggccgagaaccagaagtcctgcgagcccgctgtgcctttcccatgcggaagagtgtccgtgtctcacgccagcaagaagatcaccagagccaccaccatcttcagcaacaccgagtacgagaacttcaccgaggccgagacaatcagaggcaacgtgacccaggacgcccagagcagcgacgacttcaccagaatcgtgggcggcgagaacgctaagcccggccagttcccgtggcaggtgctgctgaacggaaaggtggaagccttctgcggcggctccatcatcaacgagaaatgggtcgtgacagccgcccactgcatcaagcccgacgacaacatcacagtggtggccggcgagtacaacatccaggaaaccgagaacacagagcagaaacggaacgtgatcaggatcatcccctaccacaagtacaacgctaccatcaacaagtataaccacgatatcgccctgctggaactggacaagcccctgaccctgaacagctacgtgacccccatctgtatcgccaacagagagtacaccaacatctttctgaacttcggcagcggctacgtgtccggatggggcagagtgttcaacagaggcagacaggccagcatcctgcagtacctgagagtgcccttcgtggacagggccacctgtctgctgagcaccaagttcaccatctacaacaacatgttctgcgccggcttcgatgtgggcggcaaggattcttgcgagggcgattctggcggccctcacgtgacagaggtggaaggcaccagctttctgaccggcatcatcagctggggcgaggaatgcgccatcaagggcaaatacggcgtgtacaccagagtgtcttggtacgtgaactggatcaaagaaaagacaaagctgaccagcgtgtcccagaccagcaagctgacaagagccgagacagtgttccccgatgtggacggctccgagcagaagctgatctccgaagaggacctggaacagaaactgatcagcgaggaagatctggaacagaagctgatttctgaagaagatctggaacagaaactgatctccgaagaggatctctga

SEQ ID NO: 20 - DNA, 1569 bp, synthetic construct
  source 1..1569: mol_type = other DNA; note = pTK1904 Rabbit FIX cDNA with human cleavage site alpha and Padua
atgcagtttctgaacaccatcatggccgagagccctggcctgatcaccatctgcctgctgggctacctgctgagcgccgagtgtaccgtgttcctggaccacgagaacgccacaaagatcctgaacagagccaagagatacaacagcggcaagctggaagagttcgtgtccggcaacctggaaagagagtgcatcgaggaaagatgcagcttcgaagaggccagagaggtgttcgagaacaccgaaaagaccaccgagttctggaagcagtacgtggacggcgaccagtgcgagagcaacccttgtctgaacggcggcagctgcaaggacgacatcaacgcctacgagtgctggtgccagtacggcttcgagggcaagaactgcgagctggacagcacctgtagcatcaagaacggcagatgcgagcagttctgccggaagaacagaaacaacaagatcatctgcagctgtaccgagggctacagactggccgagaaccagaagtcctgcgagcccgctgtgcctttcccatgcggaagagtgtccgtgtcacaaacttctaagctcacccgtgctgagactgtttttcctgatgtggactacgagaacttcaccgaggccgagacaatcagaggcaacgtgacccaggacgcccagagcagcgacgacttcaccagaatcgtgggcggcgagaacgctaagcccggccagttcccgtggcaggtgctgctgaacggaaaggtggaagccttctgcggcggctccatcatcaacgagaaatgggtcgtgacagccgcccactgcatcaagcccgacgacaacatcacagtggtggccggcgagtacaacatccaggaaaccgagaacacagagcagaaacggaacgtgatcaggatcatcccctaccacaagtacaacgctaccatcaacaagtataaccacgatatcgccctgctggaactggacaagcccctgaccctgaacagctacgtgacccccatctgtatcgccaacagagagtacaccaacatctttctgaacttcggcagcggctacgtgtccggatggggcagagtgttcaacagaggcagacaggccagcatcctgcagtacctgagagtgcccttcgtggacagggccacctgtctgctgagcaccaagttcaccatctacaacaacatgttctgcgccggcttcgatgtgggcggcaaggattcttgcgagggcgattctggcggccctcacgtgacagaggtggaaggcaccagctttctgaccggcatcatcagctggggcgaggaatgcgccatcaagggcaaatacggcgtgtacaccagagtgtcttggtacgtgaactggatcaaagaaaagacaaagctgaccagcgtgtcccagaccagcaagctgacaagagccgagacagtgttccccgatgtggacggctccgagcagaagctgatctccgaagaggacctggaacagaaactgatcagcgaggaagatctggaacagaagctgatttctgaagaagatctggaacagaaactgatctccgaagaggatctctga

SEQ ID NO: 21 - DNA, 1572 bp, synthetic construct
  source 1..1572: mol_type = other DNA; note = Rabbit FIX cDNA with human cleavage site beta and Padua
atgcagtttctgaacaccatcatggccgagagccctggcctgatcaccatctgcctgctgggctacctgctgagcgccgagtgtaccgtgttcctggaccacgagaacgccacaaagatcctgaacagagccaagagatacaacagcggcaagctggaagagttcgtgtccggcaacctggaaagagagtgcatcgaggaaagatgcagcttcgaagaggccagagaggtgttcgagaacaccgaaaagaccaccgagttctggaagcagtacgtggacggcgaccagtgcgagagcaacccttgtctgaacggcggcagctgcaaggacgacatcaacgcctacgagtgctggtgccagtacggcttcgagggcaagaactgcgagctggacagcacctgtagcatcaagaacggcagatgcgagcagttctgccggaagaacagaaacaacaagatcatctgcagctgtaccgagggctacagactggccgagaaccagaagtcctgcgagcccgctgtgcctttcccatgcggaagagtgtccgtgtctcacgccagcaagaagatcaccagagccaccaccatcttcagcaacaccgagtacgagaacttcaccgaggccgagacaatcctggacaacatcacccagagcacccagtccttcaacgacttcaccagagttgtgggcggcgaggacgctaagcccggccagttcccgtggcaggtgctgctgaacggaaaggtggaagccttctgcggcggctccatcatcaacgagaaatgggtcgtgacagccgcccactgcatcaagcccgacgacaacatcacagtggtggccggcgagtacaacatccaggaaaccgagaacacagagcagaaacggaacgtgatcaggatcatcccctaccacaagtacaacgctaccatcaacaagtataaccacgatatcgccctgctggaactggacaagcccctgaccctgaacagctacgtgacccccatctgtatcgccaacagagagtacaccaacatctttctgaacttcggcagcggctacgtgtccggatggggcagagtgttcaacagaggcagacaggccagcatcctgcagtacctgagagtgcccttcgtggacagggccacctgtctgctgagcaccaagttcaccatctacaacaacatgttctgcgccggcttcgatgtgggcggcaaggattcttgcgagggcgattctggcggccctcacgtgacagaggtggaaggcaccagctttctgaccggcatcatcagctggggcgaggaatgcgccatcaagggcaaatacggcgtgtacaccagagtgtcttggtacgtgaactggatcaaagaaaagacaaagctgaccagcgtgtcccagaccagcaagctgacaagagccgagacagtgttccccgatgtggacggctccgagcagaagctgatctccgaagaggacctggaacagaaactgatcagcgaggaagatctggaacagaagctgatttctgaagaagatctggaacagaaactgatctccgaagaggatctctga

SEQ ID NO: 22 - DNA, 1569 bp, synthetic construct
  source 1..1569: mol_type = other DNA; note = Rabbit FIX Rabbit FIX cDNA with human cleavage site alpha and cleavage site beta and Padua
atgcagtttctgaacaccatcatggccgagagccctggcctgatcaccatctgcctgctgggctacctgctgagcgccgagtgtaccgtgttcctggaccacgagaacgccacaaagatcctgaacagagccaagagatacaacagcggcaagctggaagagttcgtgtccggcaacctggaaagagagtgcatcgaggaaagatgcagcttcgaagaggccagagaggtgttcgagaacaccgaaaagaccaccgagttctggaagcagtacgtggacggcgaccagtgcgagagcaacccttgtctgaacggcggcagctgcaaggacgacatcaacgcctacgagtgctggtgccagtacggcttcgagggcaagaactgcgagctggacagcacctgtagcatcaagaacggcagatgcgagcagttctgccggaagaacagaaacaacaagatcatctgcagctgtaccgagggctacagactggccgagaaccagaagtcctgcgagcccgctgtgcctttcccatgcggaagagtgtccgtgtcacaaacttctaagctcacccgtgctgagactgtttttcctgatgtggactacgagaacttcaccgaggccgagacaatcctggacaacatcacccagagcacccagtccttcaacgacttcaccagagttgtgggcggcgaggacgctaagcccggccagttcccgtggcaggtgctgctgaacggaaaggtggaagccttctgcggcggctccatcatcaacgagaaatgggtcgtgacagccgcccactgcatcaagcccgacgacaacatcacagtggtggccggcgagtacaacatccaggaaaccgagaacacagagcagaaacggaacgtgatcaggatcatcccctaccacaagtacaacgctaccatcaacaagtataaccacgatatcgccctgctggaactggacaagcccctgaccctgaacagctacgtgacccccatctgtatcgccaacagagagtacaccaacatctttctgaacttcggcagcggctacgtgtccggatggggcagagtgttcaacagaggcagacaggccagcatcctgcagtacctgagagtgcccttcgtggacagggccacctgtctgctgagcaccaagttcaccatctacaacaacatgttctgcgccggcttcgatgtgggcggcaaggattcttgcgagggcgattctggcggccctcacgtgacagaggtggaaggcaccagctttctgaccggcatcatcagctggggcgaggaatgcgccatcaagggcaaatacggcgtgtacaccagagtgtcttggtacgtgaactggatcaaagaaaagacaaagctgaccagcgtgtcccagaccagcaagctgacaagagccgagacagtgttccccgatgtggacggctccgagcagaagctgatctccgaagaggacctggaacagaaactgatcagcgaggaagatctggaacagaagctgatttctgaagaagatctggaacagaaactgatctccgaagaggatctctga

SEQ ID NO: 23 - DNA, 1569 bp, synthetic construct
  source 1..1569: mol_type = other DNA; note = Rabbit FIX Rabbit FIX with human Cleavage site alpha and Cleavage site beta and human active peptide Padua
atgcagtttctgaacaccatcatggccgagagccctggcctgatcaccatctgcctgctgggctacctgctgagcgccgagtgtaccgtgttcctggaccacgagaacgccacaaagatcctgaacagagccaagagatacaacagcggcaagctggaagagttcgtgtccggcaacctggaaagagagtgcatcgaggaaagatgcagcttcgaagaggccagagaggtgttcgagaacaccgaaaagaccaccgagttctggaagcagtacgtggacggcgaccagtgcgagagcaacccttgtctgaacggcggcagctgcaaggacgacatcaacgcctacgagtgctggtgccagtacggcttcgagggcaagaactgcgagctggacagcacctgtagcatcaagaacggcagatgcgagcagttctgccggaagaacagaaacaacaagatcatctgcagctgtaccgagggctacagactggccgagaaccagaagtcctgcgagcccgctgtgcctttcccatgcggaagagtgtccgtgtcacaaacttctaagctcacccgtgctgagactgtttttcctgatgtggactacgtgaacagcaccgaggccgagacaatcctggacaacatcacccagagcacccagtccttcaacgacttcaccagagttgtgggcggcgaggacgctaagcccggccagttcccgtggcaggtgctgctgaacggaaaggtggaagccttctgcggcggctccatcatcaacgagaaatgggtcgtgacagccgcccactgcatcaagcccgacgacaacatcacagtggtggccggcgagtacaacatccaggaaaccgagaacacagagcagaaacggaacgtgatcaggatcatcccctaccacaagtacaacgctaccatcaacaagtataaccacgatatcgccctgctggaactggacaagcccctgaccctgaacagctacgtgacccccatctgtatcgccaacagagagtacaccaacatctttctgaacttcggcagcggctacgtgtccggatggggcagagtgttcaacagaggcagacaggccagcatcctgcagtacctgagagtgcccttcgtggacagggccacctgtctgctgagcaccaagttcaccatctacaacaacatgttctgcgccggcttcgatgtgggcggcaaggattcttgcgagggcgattctggcggccctcacgtgacagaggtggaaggcaccagctttctgaccggcatcatcagctggggcgaggaatgcgccatcaagggcaaatacggcgtgtacaccagagtgtcttggtacgtgaactggatcaaagaaaagacaaagctgaccagcgtgtcccagaccagcaagctgacaagagccgagacagtgttccccgatgtggacggctccgagcagaagctgatctccgaagaggacctggaacagaaactgatcagcgaggaagatctggaacagaagctgatttctgaagaagatctggaacagaaactgatctccgaagaggatctctga

SEQ ID NO: 24 - protein, 461 aa, Homo sapiens [sequence omitted]

SEQ ID NO: 25 - protein, 461 aa, Homo sapiens [sequence omitted]

SEQ ID NO: 26 - DNA, 2973 bp, synthetic construct
  source 1..2973: mol_type = other DNA
atgcagtttctgaacaccatcatggccgagagccctggcctgatcaccatctgcctgctgggctacctgctgagcgccgagtgtaccgtgttcctggaccacgagaacgccacaaagatcctgaacagagccaagagatacaacagcggcaagctggaagagttcgtgtccggcaacctggaaagagagtgcatcgaggaaagatgcagcttcgaagaggccagagaggtgttcgagaacaccgaaaagaccaccgagttctggaagcagtacgtggacggcgaccagtgcgagagcaacccttgtctgaacggcggcagctgcaaggacgacatcaacgcctacgagtgctggtgccagtacggcttcgagggcaagaactgcgagctggacagcacctgtagcatcaagaacggcagatgcgagcagttctgccggaagaacagaaacaacaagatcatctgcagctgtaccgagggctacagactggccgagaaccagaagtcctgcgagcccgctgtgcctttcccatgcggaagagtgtccgtgtcacaaacttctaagctcacccgtgctgagactgtttttcctgatgtggactacgagaacttcaccgaggccgagacaatcagaggcaacgtgacccaggacgcccagagcagcgacgacttcaccagaatcgtgggcggcgagaacgctaagcccggccagttcccgtggcaggtgctgctgaacggaaaggtggaagccttctgcggcggctccatcatcaacgagaaatgggtcgtgacagccgcccactgcatcaagcccgacgacaacatcacagtggtggccggcgagtacaacatccaggaaaccgagaacacagagcagaaacggaacgtgatcaggatcatcccctaccacaagtacaacgctaccatcaacaagtataaccacgatatcgccctgctggaactggacaagcccctgaccctgaacagctacgtgacccccatctgtatcgccaacagagagtacaccaacatctttctgaacttcggcagcggctacgtgtccggatggggcagagtgttcaacagaggcagacaggccagcatcctgcagtacctgagagtgcccttcgtggacagggccacctgtctgctgagcaccaagttcaccatctacaacaacatgttctgcgccggcttcgatgtgggcggcaaggattcttgcgagggcgattctggcggccctcacgtgacagaggtggaaggcaccagctttctgaccggcatcatcagctggggcgaggaatgcgccatcaagggcaaatacggcgtgtacaccagagtgtcttggtacgtgaactggatcaaagaaaagacaaagctgaccagcgtgtcccagaccagcaagctgacaagagccgagacagtgttccccgatgtggacggctccgagcagaagctgatctccgaagaggacctggaacagaaactgatcagcgaggaagatctggaacagaagctgatttctgaagaagatctggaacagaaactgatctccgaagaggatctcggcagcgacaagacccacacctgtcctccatgtcccgcccctgaactgctgggcggacctagcgtgttcctgttccccccaaagcccaaggacaccctgatgatcagccggacccccgaagtgacctgcgtggtggtggatgtgtcccacgaggaccctgaagtgaagttcaattggtatgtggatggcgtggaagtgcacaacgccaagacaaagcccagagaggaacagtacaactccacctaccgggtggtgtccgtgctgaccgtgctgcaccaggactggctgaatggcaaagagtataagtgcaaagtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctaccctagccggctggccgtggaatgggagagcaatggccagcccgagaacaattacaagaccaccccccctgtgctggacagcgacggctcattcttcctgtacagcaaactgaccgtggacaagagccggtggcagcagggcaatgtgttcagctgtagcgtgatgcacgaggccctgcacaaccactacacccagaagtctctgagcctgagccccggcaagggcggaggaagtgggggaggatctggcggcggctccgataagacacatacctgccccccttgccctgccccagagctgctgggaggcccttctgtgtttctgtttccacctaagcctaaagatacactgatgatctcccgcacacctgaagtgacatgtgtggtggtggacgtgtcacatgaagatccagaagtgaagtttaattggtacgtggacggggtggaagtgcataatgctaagaccaaaccccgggaagaacagtataacagcacatacagagtggtgtctgtgctgacagtgctgcatcaggattggctgaacgggaaagaatacaaatgtaaagtgtctaacaaagctctgcccgctcctatcgaaaagacaatctccaaggctaaaggacagcccagagaacctcaggtgtacacactgcctccatcccgcgacgagctgacaaaaaatcaggtgtcactgacatgcctcgtgaaggggttttatccatctaggctggctgtggaatgggaatccaacggacagcctgaaaacaactataagacaacacctcccgtgctggactccgatggctcattttttctgtattccaagctgactgtggataagtccagatggcagcagggaaacgtgttctcctgttctgtgatgcatgaagctctgcataatcattatacccagaaaagcctgtccctgtcccctggcaagtga

SEQ ID NO: 27 - protein, 990 aa, synthetic construct
MQFLNTIMAESPGLITICLLGYLLSAECTVFLDHENATKILNRAKRYNSGKLEEFVSGNLERECIEERCSFEEAREVFENTEKTTEFWKQYVDGDQCESNPCLNGGSCKDDINAYECWCQYGFEGKNCELDSTCSIKNGRCEQFCRKNRNNKIICSCTEGYRLAENQKSCEPAVPFPCGRVSVSQTSKLTRAETVFPDVDYENFTEAETIRGNVTQDAQSSDDFTRIVGGENAKPGQFPWQVLLNGKVEAFCGGSIINEKWVVTAAHCIKPDDNITVVAGEYNIQETENTEQKRNVIRIIPYHKYNATINKYNHDIALLELDKPLTLNSYVTPICIANREYTNIFLNFGSGYVSGWGRVFNRGRQASILQYLRVPFVDRATCLLSTKFTIYNNMFCAGFDVGGKDSCEGDSGGPHVTEVEGTSFLTGIISWGEECAIKGKYGVYTRVSWYVNWIKEKTKLTSVSQTSKLTRAETVFPDVDGSEQKLISEEDLEQKLISEEDLEQKLISEEDLEQKLISEEDLGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSRLAVEWESNGQPENNYKTTPPVLDSDGSFFLYSKLTVDKSRWQQGNVFSCSVMHEALHNHYTQKSLSLSPGKGGGSGGGSGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSRLAVEWESNGQPENNYKTTPPVLDSDGSFFLYSKLTVDKSRWQQGNVFSCSVMHEALHNHYTQKSLSLSPGK